(19) United States
(12) Patent Application Publication
(10) Pub. No.: US 2025/0177351 A1
(43) Pub. Date: Jun. 5, 2025

(54) ISOINDOLINE COMPOSITIONS AND METHODS FOR TREATING NEURODEGENERATIVE DISEASE

(71) Applicant: Cognition Therapeutics, Inc., Pittsburgh, PA (US)

(72) Inventors: Mary HAMBY, Pittsburgh, PA (US); Anthony CAGGIANO, Pittsburgh, PA (US); Lora WAYBRIGHT, Pittsburgh, PA (US)

(73) Assignee: Cognition Therapeutics, Inc., Pittsburgh, PA (US)

(21) Appl. No.: 18/995,716

(22) PCT Filed: Jul. 18, 2023
(86) PCT No.: PCT/US2023/028075

§ 371 Date: Jan. 16, 2025

Related U.S. Application Data
(60) Provisional application No. 63/390,268, filed on Jul. 18, 2022.

(51) Int. Cl. A61K 31/4035; A61K 31/496; A61K 31/5377

(52) U.S. Cl. CPC A61K 31/4035; A61K 31/496; A61K 31/5377

(57) ABSTRACT

The present invention is directed to methods of treating Alzheimer's disease in a subject in need thereof. Also disclosed are methods of identifying novel compounds that may be useful in the treatment and prevention of Alzheimer's disease. Also disclosed are methods of determining the Alzheimer's disease status of a subject.

### CROSS-REFERENCE TO RELATED APPLICATIONS

This application claims priority to U.S. Provisional Application No. 63/390,268 filed on Jul. 18, 2022, which is incorporated herein by reference.

### GOVERNMENT INTERESTS

This invention was made with government support under AG057553 and AG058660 awarded by the National Institutes of Health. The government has certain rights in the invention.

### SUMMARY

The present invention is directed to methods of treating Alzheimer's disease. Also disclosed are methods of determining the Alzheimer's disease status of a subject.

Embodiments disclosed herein are directed to methods of treating Alzheimer's disease in a subject in need thereof, comprising administering to the subject a therapeutically effective amount of a compound, or a pharmaceutically acceptable salt thereof, according to Formula I:

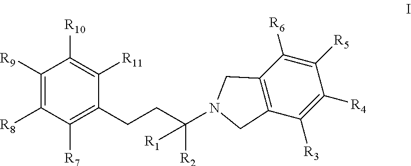

wherein:
R₁ and R₂ are each independently selected from H, C₁-C₆ alkyl, or CH₂OR′; where R′═H or C₁-C₆ alkyl;
R₃, R₄, R₅, and R₆ are each independently selected from H, C₁-C₆ alkyl, OH, OCH₃, OCH(CH₃)₂, OCH₂CH(CH₃)₂, OC(CH₃)₃, O(C₁-C₆ alkyl), OCF₃, OCH₂CH₂OH, O(C₁-C₆ alkyl)OH, O(C₁-C₆ haloalkyl), F, Cl, Br, I, CF₃, CN, NO₂, NH₂, C₁-C₆ haloalkyl, C₁-C₆ hydroxyalkyl, C₁₋₆ alkoxy C₁₋₆alkyl, aryl, heteroaryl, C₃₋₇ cycloalkyl, heterocycloalkyl, alkylaryl, heteroaryl, CO₂R′, C(O)R′, NH(C₁₋₄ alkyl), N(C₁₋₄ alkyl)₂, NH(C₃₋₇ cycloalkyl), NHC(O)(C₁₋₄ alkyl), CONR′₂, NC(O)R′, NS(O)₁R′, S(O)ₙNR′₂, S(O)ₙR′, C(O)O(C₁₋₄ alkyl), OC(O)N(R′)₂, C(O) (C₁₋₄ alkyl), and C(O)NH(C₁₋₄ alkyl); where n=0, 1, or 2; R′ are each independently H, CH₃, CH₂CH₃, C₃-C₆ alkyl, C₁-C₆ haloalkyl; or optionally substituted aryl, alkylaryl, piperazin-1-yl, piperidin-1-yl, morpholinyl, heterocycloalkyl, heteroaryl, C₁₋₆ alkoxy, NH(C₁₋₄ alkyl), or NH(C₁₋₄ alkyl)₂, wherein optionally substituted group is selected from C₁-C₆ alkyl or C₂-C₇ acyl;
or R₃ and R₄, together with the C atom to which they are attached form a form a 4-, 5-, 6- 7- or 8-membered cycloalkyl, aryl, heteroaryl, or heterocycloalkyl that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R³ and R⁴, or R⁴ and R⁵, are each independently selected from a bond, C, N, S, and O; or R₃ and R₄ are linked together to form a —O—C₁₋₂ methylene-O— group;
or R₄ and R₅, together with the C atom to which they are attached form a form a 4-, 5-, 6- 7- or 8-membered cycloalkyl, aryl, heteroaryl, or heterocycloalkyl that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R³ and R⁴, or R⁴ and R⁵, are each independently selected from a bond, C, N, S, and O; or R₄ and R₅ are linked together to form a —O—C₁₋₂ methylene-O— group;
R₇, R₈, R₉, R₁₀, and R₁₁ are each independently selected from H, C₁-C₆ alkyl, OH, OCH₃, OCH(CH₃)₂, OCH₂CH(CH₃)₂, OC(CH₃)₃, O(C₁-C₆ alkyl), OCF₃, OCH₂CH₂OH, O(C₁-C₆ alkyl)OH, O(C₁-C₆ haloalkyl), O(CO)R′, F, Cl, Br, I, CF₃, CN, NO₂, NH₂, C₁-C₆ haloalkyl, C₁-C₆ hydroxyalkyl, C₁₋₆ alkoxy C₁₋₆alkyl, aryl, heteroaryl, C₃₋₇ cycloalkyl, heterocycloalkyl, alkylaryl, heteroaryl, CO₂R′, C(O)R′, NH(C₁₋₄ alkyl), N(C₁₋₄ alkyl)₂, NH(C₃₋₇ cycloalkyl), NHC(O)(C₁₋₄ alkyl), CONR′₂, NC(O)R′, NS(O)ₙR′, S(O)ₙNR′₂, S(O)ₙR′, C(O)O(C₁₋₄ alkyl), OC(O)N(R′)₂, C(O) (C₁₋₄ alkyl), and C(O)NH(C₁₋₄ alkyl); where n=0, 1, or 2; R′ are each independently H, CH₃, CH₂CH₃, C₃-C₆ alkyl, C₁-C₆ haloalkyl, aryl, alkylaryl, piperazin-1-yl, piperidin-1-yl, morpholinyl, heterocycloalkyl, heteroaryl, C₁₋₆ alkoxy, NH(C₁₋₄ alkyl), or NH(C₁₋₄ alkyl)₂;
or R₇ and R₈, together with the N or C atoms to which they are attached form a form a 4-, 5-, 6- 7- or 8-membered cycloalkyl, aryl, heterocycloalkyl or heteroaryl group that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R⁹ and R¹⁰ are each independently selected from a bond, C, N, S, and O; or R₇ and R₈ are linked together to form a —O—C₁₋₂ methylene-O— group;
or R₈ and R₉, together with the N or C atoms to which they are attached form a form a 4-, 5-, 6- 7- or 8-membered cycloalkyl, aryl, heterocycloalkyl or heteroaryl group that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R⁹ and R¹⁰ are each independently selected from a bond, C, N, S, and O; or R₈ and R₉ are linked together to form a —O—C₁₋₂ methylene-O— group,
wherein each of the O, C₁₋₆ alkyl, C₁₋₆ haloalkyl, heteroaryl, aryl, heteroaryl, heterocycloalkyl, and cycloalkyl is optionally independently substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl and heterocycloalkyl;
with the proviso that the following compounds are excluded:

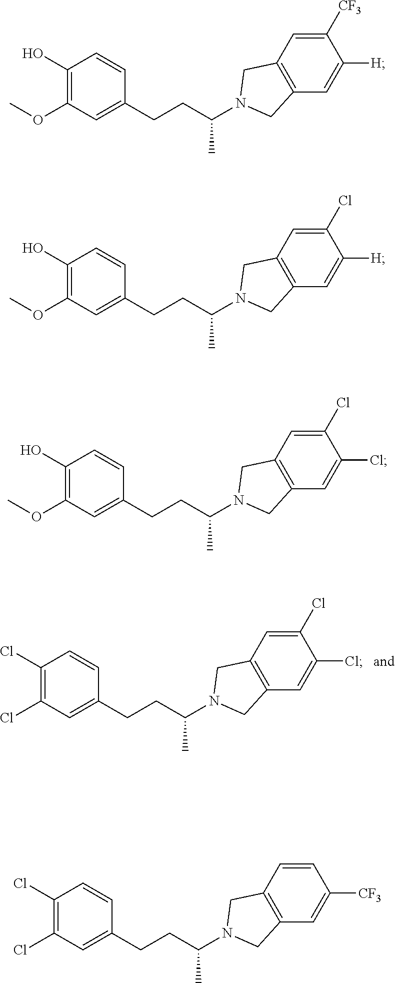

wherein the compound according to Formula I results in a decrease in the expression of at least one biomarker in the cerebral spinal fluid of the subject in need thereof, an increase in the expression of at least one biomarker in the cerebral spinal fluid of the subject in need thereof, or a combination of both,
wherein the at least one biomarker with decreased expression in the cerebral spinal fluid of the subject in need thereof is selected from the group consisting of Chitinase 3 Like 1 (CHI3L1), SPARC-Related Modular Calcium-Binding Protein 1 (SMOC 1), Clusterin (CLU), and any combination thereof.

In some embodiments, subject has been diagnosed with Alzheimer's disease. In some embodiments, the subject has been diagnosed with mild to moderate Alzheimer's disease. In some embodiments, the subject does not exhibit any detectable clinical symptoms of Alzheimer's disease. In some embodiments, the subject has an MMSE score between about 18-26. In some embodiments, the subject has an MMSE score greater than, or equal to 24.

In some embodiments, the subject is aged less than 50 years. In some embodiments, the subject is aged between 50 and 80 years.

In some embodiments, the subject has elevated cerebral spinal fluid levels of a biomarker selected from the group consisting of Chitinase 3 Like 1 (CHI3L1), SPARC-Related Modular Calcium-Binding Protein 1 (SMOC 1), Clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and any combination thereof, prior to administering the compound of Formula I.

In some embodiments, a decrease in expression of at least one biomarker selected from the group consisting of chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and any combination thereof, after administering a therapeutically effective amount of a compound of Formula I is indicative of treatment success.

In some embodiments, a decrease in cerebral spinal fluid levels of at least one biomarker selected from the group consisting of chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and any combination thereof, after administering a therapeutically effective amount of a compound of Formula I is indicative of treatment success.

In some embodiments, the compound of Formula I is administered for at least about 6 months.

In some embodiments, the compound of Formula I is

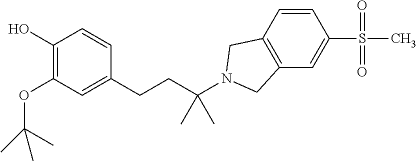 [SMILES: CC(C)(C)Oc1cc(CCC(C)(C)N2Cc3ccc(S(C)(=O)=O)cc3C2)ccc1O]

or a pharmaceutically acceptable salt thereof.

In some embodiments, the pharmaceutically acceptable salt is the fumarate salt.

In some embodiments, the therapeutically effective amount of the compound of Formula I is from about 0.0001 mg to about 1120 mg. In some embodiments, the therapeutically effective amount of the compound of Formula I is about 90 mg, 280 mg, or 560 mg.

Embodiments herein are directed to a method of treating Alzheimer's disease in a subject in need thereof, comprising administering to the subject a therapeutically effective amount of a compound, or a pharmaceutically acceptable salt thereof, according to Formula I.

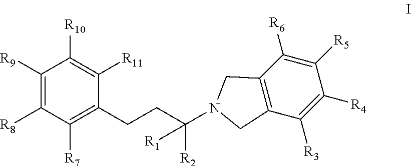

wherein:
R₁ and R₂ are each independently selected from H, C₁-C₆ alkyl, or CH₂OR′; where R′═H or C₁-C₆ alkyl;
R₃, R₄, R₅, and R₆ are each independently selected from H, C₁-C₆ alkyl, OH, OCH₃, OCH(CH₃)₂, OCH₂CH(CH₃)₂, OC(CH₃)₃, O(C₁-C₆ alkyl), OCF₃, OCH₂CH₂OH, O(C₁-C₆ alkyl)OH, O(C₁-C₆ haloalkyl), F, Cl, Br, I, CF₃, CN, NO₂, NH₂, C₁-C₆ haloalkyl, C₁-C₆ hydroxyalkyl, C₁₋₆ alkoxy C₁₋₆alkyl, aryl, heteroaryl, C₃₋₇ cycloalkyl, heterocycloalkyl, alkylaryl, heteroaryl, CO₂R′, C(O)R′, NH(C₁₋₄ alkyl), N(C₁₋₄ alkyl)₂, NH(C₃₋₇ cycloalkyl), NHC(O)(C₁₋₄ alkyl), CONR′₂, NC(O)R′, NS(O)ₙR′, S(O)ₙNR′₂, S(O)ₙR′, C(O)O(C₁₋₄ alkyl), OC(O)N(R′)₂, C(O) (C₁₋₄ alkyl), and C(O)NH(C₁₋₄ alkyl); where n=0, 1, or 2; R′ are each independently H, CH₃, CH₂CH₃, C₃-C₆ alkyl, C₁-C₆ haloalkyl; or optionally substituted aryl, alkylaryl, piperazin-1-yl, piperidin-1-yl, morpholinyl, heterocycloalkyl, heteroaryl, C₁₋₆ alkoxy, NH(C₁₋₄ alkyl), or NH(C₁₋₄ alkyl)₂, wherein optionally substituted group is selected from C₁-C₆ alkyl or C₂-C₇ acyl;
or R₃ and R₄, together with the C atom to which they are attached form a 4-, 5-, 6- 7- or 8-membered cycloalkyl, aryl, heteroaryl, or heterocycloalkyl that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R³ and R⁴, or R⁴ and R⁵, are each independently selected from a bond, C, N, S, and O; or R₃ and R₄ are linked together to form a —O—C₁₋₂ methylene-O— group; or R₄ and R₅, together with the C atom to which they are attached form a 4-, 5-, 6- 7- or 8-membered cycloalkyl, aryl, heteroaryl, or heterocycloalkyl that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R³ and R⁴, or R⁴ and R⁵, are each independently selected from a bond, C, N, S, and O; or R₄ and R₅ are linked together to form a —O—C₁₋₂ methylene-O— group;
R₇, R₈, R₉, R₁₀, and R₁₁ are each independently selected from H, C₁-C₆ alkyl, OH, OCH₃, OCH(CH₃)₂, OCH₂CH(CH₃)₂, OC(CH₃)₃, O(C₁-C₆ alkyl), OCF₃, OCH₂CH₂OH, O(C₁-C₆ alkyl)OH, O(C₁-C₆ haloalkyl), O(CO)R′, F, Cl, Br, I, CF₃, CN, NO₂, NH₂, C₁-C₆ haloalkyl, C₁-C₆ hydroxyalkyl, C₁₋₆ alkoxy C₁₋₆alkyl, aryl, heteroaryl, C₃₋₇ cycloalkyl, heterocycloalkyl, alkylaryl, heteroaryl, CO₂R′, C(O)R′, NH(C₁₋₄ alkyl), N(C₁₋₄ alkyl)₂, NH(C₃₋₇ cycloalkyl), NHC(O)(C₁₋₄ alkyl), CONR′₂, NC(O)R′, NS(O)ₙR′, S(O)ₙNR′₂, S(O)ₙR′, C(O)O(C₁₋₄ alkyl), OC(O)N(R′)₂, C(O) (C₁₋₄ alkyl), and C(O)NH(C₁₋₄ alkyl); where n=0, 1, or 2; R′ are each independently H, CH₃, CH₂CH₃, C₃-C₆ alkyl, C₁-C₆ haloalkyl, aryl, alkylaryl, piperazin-1-yl, piperidin-1-yl, morpholinyl, heterocycloalkyl, heteroaryl, C₁₋₆ alkoxy, NH(C₁₋₄ alkyl), or NH(C₁₋₄ alkyl)₂;
or R₇ and R₈, together with the N or C atoms to which they are attached form a 4-, 5-, 6- 7- or 8-membered cycloalkyl, aryl, heterocycloalkyl or heteroaryl group that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R⁹ and R¹⁰ are each independently selected from a bond, C, N, S, and O; or R₇ and R₈ are linked together to form a —O—C₁₋₂ methylene-O— group;
or R₈ and R₉, together with the N or C atoms to which they are attached form a form a 4-, 5-, 6- 7- or 8-membered cycloalkyl, aryl, heterocycloalkyl or heteroaryl group that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R⁹ and R¹⁰ are each independently selected from a bond, C, N, S, and O; or R₈ and R₉ are linked together to form a —O—C₁₋₂ methylene-O— group,
wherein each of the O, C₁₋₆ alkyl, C₁₋₆ haloalkyl, heteroaryl, aryl, heteroaryl, heterocycloalkyl, and cycloalkyl is optionally independently substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl and heterocycloalkyl;
with the proviso that the following compounds are excluded:

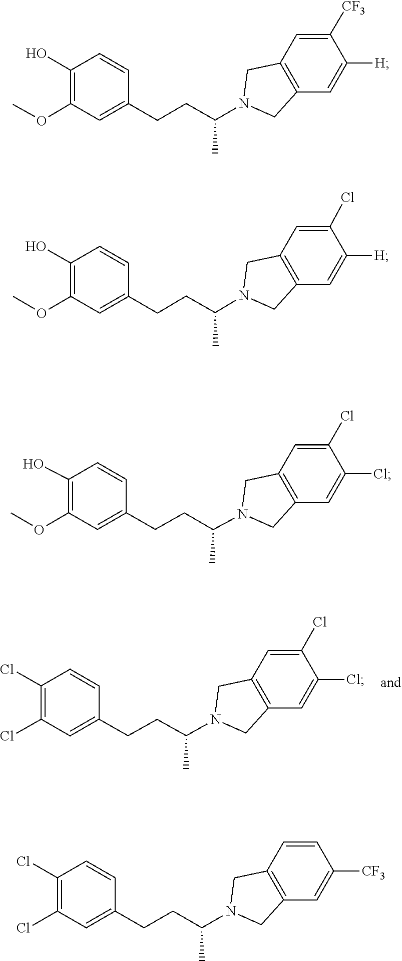

wherein the subject in need thereof, prior to administration of the compound of Formula I, has a decrease in the expression of at least one biomarker of the cerebral spinal fluid of the subject in need thereof, an increase in the expression of at least one biomarker in the cerebral spinal fluid of the subject in need thereof, or a combination of both;
wherein the at least one biomarker with decreased expression is selected from the group consisting of chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and any combination thereof.

### BRIEF DESCRIPTION OF THE FIGURES

FIG. 1 depicts the design of the phase 1b/2a clinical trial.

FIG. 2 depicts Aβ, tau, and NFL levels before and after treatment with CT812.

FIG. 3 depicts neurogranin (synaptic damage marker elevated in Alzheimer's CSF) reduced by 33% in 90 mg dose group and 17.6% in the pooled CT1812-treated group. Reduction consistent with a positive effect on synapses, CT1812's mechanism of action, and preclinical studies.

FIG. 4 depicts the effect of Aβ oligomer treatment on full length neurogranin protein expression in neurons. Neurogranin levels were decreased consistent with increase in cleaved, secreted forms of neurogranin in Alzheimer's CSF. Aβ42 oligomer exposure restores neurogranin expression consistent with positive effect on synapses. Aβ42 oligomers were added to mature (21 days in vitro) hippocampal/cortical cultures (500 nM, 1 hr) followed by CT1812 (4.8 nM) or vehicle treatment (24 hr).

FIGS. 5A and 5B depict various protein expression levels CT1812 for 28 days versus placebo in CSF and plasma.

FIGS. 6A and 6B depicts 30 proteins changed differentially in CT1812-treated vs. placebo patients (p≤0.05, i.e., higher or lower expression vs. placebo). Several play key roles in synaptic plasticity and are dysregulated in Alzheimer's disease brain: synaptotagmin-1, a synaptic damage marker elevated in Alzheimer's CSF. Expression decreased 63% in CT1812-treated vs. placebo consistent with positive effect on synapses and CT1812's mechanism of action.

FIGS. 7A and 7B depict the displacement of Aβ oligomers from neurons, from Alzheimer's disease patient neurocortical tissue, from the hippocampus of living transgenic APP/PS1 mice (where Aβ oligos are cleared into the CSF without affecting monomer concentrations. Also shown is the restoration of synapse number and memory in transgenic Alzheimer's disease mice at concentrations of greater than 80% receptor occupancy.

FIG. 8 depicts exploratory cognitive outcomes from the phase 1b/2a clinical trial.

FIG. 9 depicts CT1812 pharmacokinetic parameters in the plasma (day 28) and CSF (days 22-30).

FIG. 10 depicts plasma concentrations of CT1812 following a single oral dose (SAD) or after Q.D. dosing for 3 or 14 days (MAD) in healthy young and elderly subjects. Plasma concentrations increases were slightly greater than proportional with dose and exhibited minimal accumulation with repeat dosing.

FIG. 11 depicts increase in CT1812 concentrations in cerebrospinal fluid (CSF) in a dose-dependent manner. In the 560 mg-840 mg dose range, average CSF concentrations (at the approximate plasma Tₘₐₓ, 1.5 hr post-dose) were equivalent to those in mice that have a 97-98% receptor occupancy concentration in the brain. Horizontal lines indicate projected receptor occupancy based on concentration and receptor occupancy determined in pre-clinical mouse Alzheimer's disease models.

FIG. 12 depicts biomarkers that are upregulated or downregulated in patients with Alzheimer's disease.

FIG. 13 depicts levels of canonical Alzheimer's disease biomarkers detected by proteomics.

FIG. 14 depicts correlations between TMT-MS values and clinically validated quantitative assays.

FIG. 15 depicts differential expression analysis between cerebral spinal fluid from patients in treatment vs placebo.

### DETAILED DESCRIPTION

Before compounds, compositions and methods are described in detail, it is to be understood that this disclosure is not limited to the particular processes, compositions, or methodologies described, as these may vary. It is also to be understood that the terminology used in the description is for the purpose of describing the particular versions or embodiments only, and is not intended to limit the scope of the disclosure which will be limited only by the appended claims. Unless defined otherwise, all technical and scientific terms used herein have the same meaning as commonly understood by one of ordinary skill in the art. Although any methods and materials similar or equivalent to those described herein can be used in the practice or testing of embodiments of the disclosure, the preferred methods, devices, and materials are now described.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

### Definitions

The singular forms “a”, “an”, and “the” include plural reference unless the context clearly dictates otherwise. Thus, for example, reference to a “cell” is a reference to one or more cells and equivalents thereof known to those skilled in the art, and so forth.

As used herein, the term “about” as used herein, means plus or minus 10% of a given value. For example, “about 50%” means in the range of 45%-55%.

The term “amyloid beta peptide”, “Aβ,” or “Abeta” as used herein, refers to extracellular amyloid beta protein that is produced through the proteolytic processing of a transmembrane protein, amyloid precursor protein (APP), by β- and γ-secretases. Alzheimer's disease (AD) is defined histologically by the presence of Aβ in the brain.

The term “agonist” as used herein, refers to a compound, the presence of which results in a biological activity of a receptor that is the same as the biological activity resulting from the presence of a naturally occurring ligand for the receptor.

The term “partial agonist” as used herein, refers to a compound the presence of which results in a biological activity of a receptor that is of the same type as that resulting from the presence of a naturally occurring ligand for the receptor, but of a lower magnitude.

The term “antagonist” as used herein, refers to an entity, e.g., a compound, antibody or fragment, the presence of which results in a decrease in the magnitude of a biological activity of a receptor. In certain embodiments, the presence of an antagonist results in complete inhibition of a biological activity of a receptor. As used herein, the term “sigma-2 receptor antagonist” is used to describe a compound that acts as a “functional antagonist” at the sigma-2 receptor in that it blocks Abeta effects, for example, Abeta oligomer-induced synaptic dysfunction, for example, as seen in an in vitro assay, such as a membrane trafficking assay, or a synapse loss assay, or Abeta oligomer mediated sigma-2 receptor activation of caspase-3, or in a behavioral assay, or in a patient in need thereof. The functional antagonist may act directly by inhibiting binding of, for example, an Abeta oligomer to a sigma-2 receptor, or indirectly, by interfering with downstream signaling resultant from Abeta oligomer binding the sigma-2 receptor.

The term “modulator” as used herein, refers to an entity, e.g., a compound, antibody, or fragment, a substance, endogenous or exogenous, that binds to and regulates the activity of a substrate such as a receptor. A modulator can be a mixed agonist-antagonist. A modulator can be an antagonist. A modulator can be an agonist.

The term “biomarker” as used herein, shall mean an organic biomolecule which is differentially present in a sample taken from a subject of one phenotypic status (e.g. having a disease) as compared with another phenotypic status (e.g., not having the disease). A biomarker is differentially present between different phenotypic statuses if the mean or median expression level of the biomarker in the different groups is calculated to be statistically significant. “Increased expression” as used herein refers to an increased amount of a biomarker in one group compared to another, in this case the biomarker may be present at “elevated levels.” “Decreased expression” as used herein refers to a decreased amount of a biomarker in one group compared to another; in this case the biomarker may be present in at “decreased levels.” Common tests for statistical significance include, but are not limited to, t-test, ANOVA, Kruskal-Wallis, Wilcoxon, Mann-Whitney and odds ratio. Biomarkers, alone or in combination, provide measures of relative risk that a subject belongs to one phenotypic status or another. As such, they are useful as markers for disease, therapeutic effectiveness of a drug, or drug candidate and of drug toxicity. In some embodiments, the biomarker is a protein. In some embodiments, the biomarker is ribonucleic acid (RNA).

The term “sigma-2 receptor antagonist” as used herein, refers to a molecule that binds to a sigma-2 receptor in a measurable amount and acts as a functional antagonist with respect to Abeta effects oligomer induced synaptic dysfunction resultant from sigma-2 receptor binding.

The term “sigma-2 receptor modulator” as used herein, refers to a molecule that binds to a sigma-2 receptor in a measurable amount and acts to modulate the function of the sigma-2 receptor.

The term “sigma-2 ligand” as used herein, refers to a compound that binds to a sigma-2 receptor and includes agonists, antagonists, partial agonists, inverse agonists and simply competitors for other ligands of this receptor or protein.

The term “selectivity” or “selective” as used herein, refers to a difference in the binding affinity of a compound (Kᵢ) for a sigma receptor, for example, a sigma-2 receptor, compared to a non-sigma receptor. The sigma-2 modulators possess high selectivity for a sigma receptor in synaptic neurons. The Kᵢ for a sigma-2 receptor or both a sigma-2 and a sigma-1 receptor is compared to the Kᵢ for a non-sigma receptor. In some embodiments, the selective sigma-2 receptor modulator, or sigma-1 receptor ligand, has at least 10-fold, 20-fold, 30-fold, 50-fold, 70-fold, 100-fold, or 500-fold higher affinity, or more, for binding to a sigma receptor compared to anon-sigma receptor as assessed by a comparison of binding dissociation constant Kᵢ values, or IC₅₀ values, or binding constant, at different receptors. Any known assay protocol can be used to assess the Kᵢ or IC₅₀ values at different receptors, for example, by monitoring the competitive displacement from receptors of a radiolabeled compound with a known dissociation constant, for example, by the method of Cheng and Prusoff (1973) (Biochem. Pharmacol. 22, 3099-3108), or specifically as provided herein. In some embodiments, the sigma-2 modulator compound is an antibody, or active binding fragment thereof, specific for binding to a sigma-2 receptor compared to a non-sigma receptor. In the case of an antibody, or fragment, binding constants at a sigma-2 receptor, or fragment, can be calculated and compared to binding constants at a non-sigma receptor by any means known in the art, for example, by the method of Beatty et al., 1987, J Immunol Meth, 100(1-2):173-179, or the method of Chalquest, 1988, J. Clin. Microbiol. 26(12): 2561-2563. The non-sigma receptor is, for example, selected from a muscarinic M1-M4 receptor, serotonin (5-HT) receptor, alpha adrenergic receptor, beta adrenergic receptor, opioid receptor, serotonin transporter, dopamine transporter, adrenergic transporter, dopamine receptor, or NMDA receptor.

The term “high affinity” as used herein, is intended to mean a compound which exhibits a Kᵢ value of less than 600 nM, 500 nM, 400 nM, 300 nM, 200 nM, less than 150 nM, less than 100 nM, less than 80 nM, less than 60 nM, or preferably less than 50 nM in a sigma receptor binding assay, for example against [³H]-DTG, as disclosed by Weber et al., Proc. Natl. Acad. Sci (USA) 83: 8784-8788 (1986), incorporated herein by reference, which measures the binding affinity of compounds toward both the sigma-1 and sigma-2 receptor sites. Especially preferred sigma ligands exhibit Kᵢ values of less than about 150 nM, preferably less than 100 nM, less than about 60 nM, less than about 10 nM, or less than about 1 nM against [³H]-DTG.

The term “therapeutic profile” as used herein, refers to a compound that meets the therapeutic phenotype, and also has good brain penetrability (the ability to cross the blood brain barrier), good plasma stability and good metabolic stability.

The term “drug-like properties” as used herein, refers to the pharmacokinetic and stability characteristics of the sigma-2 receptor ligands upon administration; including brain penetrability, metabolic stability and/or plasma stability.

The term “administering,” when used in conjunction with the compounds of the disclosure, means to administer a compound directly into or onto a target tissue or to administer a compound systemically or locally to a patient or other subject.

The term “animal” as used herein includes, but is not limited to, humans and non-human vertebrates such as wild, experimental, domestic and farm animals and pets.

The terms “subject,” “individual,” and “patient,” as used herein are interchangeable and refer to any animal, including mammals, mice, rats, other rodents, rabbits, dogs, cats, swine, cattle, sheep, horses, primates, non-human primates, humans, and the like. In some embodiments the term subject refers to a mammalian cell.

the term “contacting” as used herein, refers to the bringing together or combining of molecules (or of a molecule with a higher order structure such as a cell or cell membrane) such that they are within a distance that allows for intermolecular interactions such as the non-covalent interaction between two peptides or one protein and another protein or other molecule, such as a small molecule. In some embodiments, contacting occurs in a solution in which the combined or contacted molecules are mixed in a common solvent and are allowed to freely associate. In some embodiments, the contacting can occur at or otherwise within a cell or in a cell-free environment. In some embodiments, the cell-free environment is the lysate produced from a cell. In some embodiments, a cell lysate may be a whole-cell lysate, nuclear lysate, cytoplasm lysate, and combinations thereof. In some embodiments, the cell-free lysate is lysate obtained from a nuclear extraction and isolation wherein the nuclei of a cell population are removed from the cells and then lysed. In some embodiments, the nuclei are not lysed, but are still considered to be a cell-free environment. The molecules can be brought together by mixing such as vortexing, shaking, and the like.

The term “improves” as used herein, conveys that the disclosure changes either the characteristics and/or the physical attributes of the tissue to which it is being provided, applied or administered. The term “improves” may also be used in conjunction with a disease state such that when a disease state is “improved” the symptoms or physical characteristics associated with the disease state are diminished, reduced, eliminated, delayed or averted.

The term “inhibiting” as used herein, includes the blockade, aversion of a certain result or process, or the restoration of the converse result or process. In terms of prophylaxis or treatment by administration of a compound of the disclosure, “inhibiting” includes protecting against (partially or wholly) or delaying the onset of symptoms, alleviating symptoms, or protecting against, diminishing or eliminating a disease, condition or disorder.

The term “log P” as used herein, refers to the partition coefficient of a compound. The partition coefficient is the ratio of concentrations of un-ionized compound in each of two solution phases, for example, octanol and water. To measure the partition coefficient of ionizable solute compounds, the pH of the aqueous phase is adjusted such that the predominant form of the compound is un-ionized. The logarithm of the ratio of concentrations of the un-ionized solute compound in the solvents is called log P. The log P is a measure of lipophilicity. For example,

log P_{oct/wat}=log([solute]_{octanol}/[solute]_{un-ionized, water}).

At various places in the present specification, substituents of compounds of the disclosure are disclosed in groups or in ranges. It is specifically intended that embodiments of the disclosure include each and every individual subcombination of the members of such groups and ranges. For example, the term “C₁₋₆ alkyl” is specifically intended to individually disclose e.g. methyl (C₁ alkyl), ethyl (C₂ alkyl), C₃ alkyl, C₄ alkyl, C₅ alkyl, and C₆ alkyl as well as, e.g. C₁-C₂ alkyl, C₁-C₃ alkyl, C₁-C₄ alkyl, C₂-C₃ alkyl, C₂-C₄ alkyl, C₃-C₆ alkyl, C₄-C₅ alkyl, and C₅-C₆ alkyl.

For compounds of the disclosure in which a variable appears more than once, each variable can be a different moiety selected from the Markush group defining the variable. For example, where a structure is described having two R groups that are simultaneously present on the same compound, then the two R groups can represent different moieties selected from the Markush group defined for R.

The term “n-membered” where n is an integer typically describes the number of ring-forming atoms in a moiety where the number of ring-forming atoms is n. For example, pyridine is an example of a 6-membered heteroaryl ring and thiophene is an example of a 5-membered heteroaryl group.

The term “hydrogen bond acceptor group” as used herein, refers to a group capable of accepting a hydrogen bond. Examples of hydrogen bond acceptor groups are known and include, but are not limited to, alkoxy groups, oxazolidin-2-one groups, —O—C(O)—N—; —C(O)—N—; —O—; the hetero atom (e.g. oxygen) in a cycloheteroalkyl; —N—SO₂— and the like. The groups can be bound in either direction and can be connected to another carbon or heteroatom. A hydrogen bond acceptor group can also be present in or near a hydrophobic aliphatic group. For example, a tetrahydrofuran group comprises both a hydrogen bond acceptor group and a hydrophobic aliphatic group. The oxygen present in the tetrahydrofuran ring acts as a hydrogen bond acceptor and the carbons in the tetrahydrofuran ring act as the hydrophobic aliphatic group.

The term “hydrophobic aliphatic group” as used herein, refers to a carbon chain or carbon ring. The carbon chain can be present in a cycloheteroalkyl, but the hydrophobic aliphatic group does not include the heteroatom. The tetrahydrofuran example provided above is one such example, but there are many others. In some embodiments, the hydrophobic aliphatic group is an optionally substituted C₁-C₆ alkyl, cycloalkyl, or C₁-C₆ carbons of a heterocycloalkyl. A “hydrophobic aliphatic group” is not a hydrophobic aromatic group.

The term “positive ionizable group” as used herein, refers to an atom or a group of atoms present in a structure that can be positively charged under certain conditions such as biological conditions present in solution or in a cell. In some embodiments, the positive ionizable group is a nitrogen. In some embodiments, the positive ionizable group is a nitrogen present in a cycloheteroalkyl ring. For example, in a piperazine group, the two nitrogens would be considered two positive ionizable groups. However, in some embodiments, the carbons linked to a positive ionizable group are not considered a hydrophobic aliphatic group. In some embodiments, the positive ionizable group is a nitrogen containing ring. Examples of nitrogen containing rings include, but are not limited to, piperazine, piperadine, triazinane, tetrazinane, and the like. In some embodiments with respect to the positive ionizable group, a nitrogen containing ring comprises 1, 2, 3, or 4 nitrogens. In some embodiments, the positive ionizable group is not the nitrogen present in a —N—SO₂— group

In some embodiments, a group comprises both a hydrogen bond acceptor and a positive ionizable group. For example, a morpholine group comprises both a hydrogen bond acceptor in the oxygen group and a positive ionizable group in the nitrogen.

The term “hydrogen bond donor” as used herein, refers to a group that is capable of donating a hydrogen bond. Examples of a hydrogen bond donor group include, but are not limited to, —OH, and the like.

The term “alkyl” as used herein, refers to a saturated hydrocarbon group which is straight-chained or branched. Example alkyl groups include, but are not limited to, methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 20, from 2 to about 20, from 1 to about 10, from 1 to about 8, from 1 to about 6, from 1 to about 4, or from 1 to about 3 carbon atoms. The term “alkylene” refers to a divalent alkyl linking group. An example of alkylene is methylene (CH₂).

The term “alkenyl” as used herein, refers to an alkyl group having one or more double carbon-carbon bonds. Example alkenyl groups include, but are not limited to, ethenyl, propenyl, cyclohexenyl, and the like. The term “alkenylenyl” refers to a divalent linking alkenyl group.

The term “alkynyl” as used herein, refers to an alkyl group having one or more triple carbon-carbon bonds. Example alkynyl groups include, but are not limited to, ethynyl, propynyl, and the like. The term “alkynylenyl” refers to a divalent linking alkynyl group.

The term “haloalkyl” as used herein, refers to an alkyl group having one or more halogen substituents selected from F, Cl, Br, and/or I. Example haloalkyl groups include, but are not limited to, CF₃, C₂F₅, CHF₂, CCl₃, CHCl₂, C₂Cl₅, CH₂CF₃, and the like.

The term “aryl” as used herein, refers to monocyclic or polycyclic (e.g., having 2, 3 or 4 fused rings) aromatic hydrocarbons such as, for example, phenyl, naphthyl, anthracenyl, phenanthrenyl, indanyl, indenyl, and the like. In some embodiments, aryl groups have from 6 to about 20 carbon atoms. In some embodiments, aryl groups have from 6 to about 10 carbon atoms.

The term “cycloalkyl” as used herein, refers to non-aromatic cyclic hydrocarbons including cyclized alkyl, alkenyl, and alkynyl groups that contain up to 20 ring-forming carbon atoms. Cycloalkyl groups can include mono- or polycyclic (e.g., having 2, 3 or 4 fused rings) ring systems as well as spiro ring systems. A cycloalkyl group can contain from 3 to about 15, from 3 to about 10, from 3 to about 8, from 3 to about 6, from 4 to about 6, from 3 to about 5, or from 5 to about 6 ring-forming carbon atoms. Ring-forming carbon atoms of a cycloalkyl group can be optionally substituted by oxo or sulfido. Example of cycloalkyl groups include, but are not limited to, cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (i.e., having a bond in common with) to the cycloalkyl ring, for example, benzo or thienyl derivatives of pentane, pentene, hexane, and the like (e.g., 2,3-dihydro-1H-indene-1-yl, or 1H-inden-2(3H)-one-1-yl). Preferably, “cycloalkyl” refers to cyclized alkyl groups that contain up to 20 ring-forming carbon atoms. Examples of cycloalkyl preferably include cyclopropyl, cyclobutyl, cyclopentyl, cyclohexyl, cycloheptyl, adamantyl, and the like.

The term “heteroaryl groups” as used herein, refers to an aromatic heterocycle having up to 20 ring-forming atoms and having at least one heteroatom ring member (ring-forming atom) such as sulfur, oxygen, or nitrogen. In some embodiments, the heteroaryl group has at least one or more heteroatom ring-forming atoms each independently selected from sulfur, oxygen, and nitrogen. Heteroaryl groups include monocyclic and polycyclic (e.g., having 2, 3 or 4 fused rings) systems. Examples of heteroaryl groups include without limitation, pyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 1 to about 5, from about 1 to about 4, from about 1 to about 3, from about 1 to about 2, carbon atoms as ring-forming atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

The term “heterocycloalkyl” as used herein, refers to non-aromatic heterocycles having up to 20 ring-forming atoms including cyclized alkyl, alkenyl, and alkynyl groups where one or more of the ring-forming carbon atoms is replaced by a heteroatom such as an O, N, or S atom. Heterocycloalkyl groups can be mono or polycyclic (e.g., both fused and spiro systems).

Example “heterocycloalkyl” groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, pyrrolidin-2-one-3-yl, and the like. Ring-forming carbon atoms and heteroatoms of a heterocycloalkyl group can be optionally substituted by oxo or sulfido. For example, a ring-forming S atom can be substituted by 1 or 2 oxo [i.e., form a S(O) or S(O)₂]. For another example, a ring-forming C atom can be substituted by oxo (i.e., form carbonyl). Also included in the definition of heterocycloalkyl are moieties that have one or more aromatic rings fused (i.e., having a bond in common with) to the nonaromatic heterocyclic ring, for example pyridinyl, thiophenyl, phthalimidyl, naphthalimidyl, and benzo derivatives of heterocycles such as indoline, isoindoline, isoindolin-1-one-3-yl, 4,5,6,7-tetrahydrothieno[2,3-c]pyridine-5-yl, 5,6-dihydrothieno[2,3-c]pyridin-7(4H)-one-5-yl, and 3,4-dihydroisoquinolin-1(2H)-one-3yl groups. Ring-forming carbon atoms and heteroatoms of the heterocycloalkyl group can be optionally substituted by oxo or sulfido. In some embodiments, the heterocycloalkyl group has from 1 to about 20 carbon atoms, and in further embodiments from about 3 to about 20 carbon atoms. In some embodiments, the heterocycloalkyl group contains 3 to about 14, 3 to about 7, or 5 to 6 ring-forming atoms. In some embodiments, the heterocycloalkyl group has 1 to about 4, 1 to about 3, or 1 to 2 heteroatoms. In some embodiments, the heterocycloalkyl group contains 0 to 3 double bonds.

In some embodiments, the heterocycloalkyl group contains 0 to 2 triple bonds.

The terms “halo” or “halogen” as used herein, includes fluoro, chloro, bromo, and iodo.

The term “alkoxy” as used herein, refers to an —O-alkyl group. Example alkoxy groups include methoxy, ethoxy, propoxy (e.g., n-propoxy and isopropoxy), t-butoxy, and the like.

The term “haloalkoxy” as used herein, refers to an —O-haloalkyl group. An example haloalkoxy group is OCF₃. As used herein, “trihalomethoxy” refers to a methoxy group having three halogen substituents. Examples of trihalomethoxy groups include, but are not limited to, —OCF₃, —OCClF₂, —OCCl₃, and the like.

The term “arylalkyl” as used herein, refers to a C₁₋₆ alkyl substituted by aryl and “cycloalkylalkyl” refers to C₁₋₆ alkyl substituted by cycloalkyl.

The term “heteroarylalkyl” as used herein, refers to a C₁₋₆ alkyl group substituted by a heteroaryl group, and “heterocycloalkylalkyl” refers to a C₁₋₆ alkyl substituted by heterocycloalkyl.

The term “amino” as used herein, refers to NH₂.

The term “alkylamino” as used herein, refers to an amino group substituted by an alkyl group.

The term “dialkylamino” as used herein, refers to an amino group substituted by two alkyl groups.

The term “C(O)” as used herein, refers to C(═O).

The term “optionally substituted” as used herein, means that substitution is optional and therefore includes both unsubstituted and substituted atoms and moieties. A “substituted” atom or moiety indicates that any hydrogen on the designated atom or moiety can be replaced with a selection from the indicated substituent group, provided that the normal valence of the designated atom or moiety is not exceeded, and that the substitution results in a stable compound. For example, if a methyl group (i.e., CH₃) is optionally substituted, then 3 hydrogen atoms on the carbon atom can be replaced with substituent groups, in indicated.

The term “salts” as used herein, includes acid addition salts or addition salts of free bases. Preferably, the salts are pharmaceutically acceptable. Examples of acids which may be employed to form pharmaceutically acceptable acid addition salts include, but are not limited to, salts derived from nontoxic inorganic acids such as nitric, phosphoric, sulfuric, or hydrobromic, hydroiodic, hydrofluoric, phosphorous, as well as salts derived from nontoxic organic acids such as aliphatic mono- and dicarboxylic acids, phenyl-substituted alkanoic acids, hydroxyl alkanoic acids, alkanedioic acids, aromatic acids, aliphatic and aromatic sulfonic acids, and acetic, maleic, succinic, or citric acids. Non-limiting examples of such salts include napadisylate, besylate, sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate (see, for example, Berge, et al. “Pharmaceutical Salts,” J. Pharma. Sci. 1977; 66:1).

The term “pharmaceutically acceptable” as used herein, refers to molecular entities and compositions that are generally regarded as safe and nontoxic. In particular, pharmaceutically acceptable carriers, diluents or other excipients used in the pharmaceutical compositions of this disclosure are physiologically tolerable, compatible with other ingredients, and do not typically produce an allergic or similar untoward reaction (for example, gastric upset, dizziness and the like) when administered to a patient. Preferably, as used herein, the term “pharmaceutically acceptable” means approved by a regulatory agency of the Federal or a state government or listed in the U.S. Pharmacopoeia or other generally recognized pharmacopoeia for use in animals, and more particularly in humans. The phrase “pharmaceutically acceptable salt(s)”, as used herein, includes those salts of compounds of the disclosure that are safe and effective for use in mammals and that possess the desired biological activity. Pharmaceutically acceptable salts include salts of acidic or basic groups present in compounds of the disclosure or in compounds identified pursuant to the methods of the disclosure. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the disclosure can form pharmaceutically acceptable salts with various amino acids. Suitable base salts include, but are not limited to, aluminum, calcium, lithium, magnesium, potassium, sodium, zinc, iron and diethanolamine salts. Pharmaceutically acceptable base addition salts are also formed with amines, such as organic amines. Examples of suitable amines are N,N′-dibenzylethylenediamine, chloroprocaine, choline, diethanolamine, dicyclohexylamine, ethylenediamine, N-methylglucamine, and procaine.

The term “therapeutic” as used herein, means an agent utilized to treat, combat, ameliorate, protect against or improve an unwanted condition or disease of a subject.

The term “active ingredient” as used herein, refers to a compound of any of the formulae defined herein.

The term “metabolite” as used herein, refers to a derivative of a compound which is formed when the compound is metabolized. The term “active metabolite” as used herein, refers to a biologically active derivative of a compound which is formed when the compound is metabolized. The term “metabolized” as used herein, refers to the sum of the processes by which a particular substance is changed in the living body. In brief, all compounds present in the body are manipulated by enzymes within the body in order to derive energy and/or to remove them from the body. Specific enzymes produce specific structural alterations to the compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Further information on metabolism may be obtained from The Pharmacological Basis of Therapeutics, 9^{th} Edition, McGraw-Hill (1996), pages 11-17. Metabolites of the compounds disclosed herein can be identified either by administration of compounds to a host and analysis of tissue samples from the host, or by incubation of compounds with hepatic cells in vitro and analysis of the resulting compounds. Both methods are well known in the art.

The term “effective amount” as used herein, refers to an amount that results in measurable inhibition of at least one symptom or parameter of a specific disorder or pathological process. For example, an amount of a sigma-2 ligand of the disclosure that provides a measurably lower synapse reduction in the presence of Abeta oligomer qualifies as an effective amount because it reduces a pathological process even if no clinical symptoms of amyloid pathology are altered, at least immediately.

The terms “therapeutically effective amount” or “effective amount” of a compound or composition of the disclosure as used herein, refers to a predetermined amount which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect or physician observes a change). An effective amount of a compound of the disclosure may broadly range from about 0.01 mg/Kg to about 500 mg/Kg, about 0.1 mg/Kg to about 400 mg/Kg, about 1 mg/Kg to about 300 mg/Kg, about 0.05 to about 20 mg/Kg, about 0.1 mg/Kg to about 10 mg/Kg, or about 10 mg/Kg to about 100 mg/Kg. The effect contemplated herein includes both medical therapeutic and/or prophylactic treatment, as appropriate. The specific dose of a compound administered according to this disclosure to obtain therapeutic and/or prophylactic effects will, of course, be determined by the particular circumstances surrounding the case, including, for example, the compound administered, the route of administration, the co-administration of other active ingredients, the condition being treated, the activity of the specific compound employed, the specific composition employed, the age, body weight, general health, sex and diet of the patient; the time of administration, route of administration, and rate of excretion of the specific compound employed and the duration of the treatment. The effective amount administered will be determined by the physician in the light of the foregoing relevant circumstances and the exercise of sound medical judgment. A therapeutically effective amount of a compound of this disclosure is typically an amount such that when it is administered in a physiologically tolerable excipient composition, it is sufficient to achieve an effective systemic concentration or local concentration in the tissue. The total daily dose of the compounds of this disclosure administered to a human or other animal in single or in divided doses can be in amounts, for example, from 0.01 mg/Kg to about 500 mg/Kg, about 0.1 mg/Kg to about 400 mg/Kg, about 1 mg/Kg to about 300 mg/Kg, about 10 mg/Kg to about 100 mg/Kg, or more usually from 0.1 to 25 mg/kg body weight per day. Single dose compositions may contain such amounts or submultiples thereof to make up the daily dose. In general, treatment regimens according to the disclosure comprise administration to a patient in need of such treatment will usually include from about 1 mg to about 5000 mg, 10 mg to about 2000 mg of the compound(s), 20 to 1000 mg, preferably 20 to 500 mg and most preferably about 50 mg or 100 mg or 300 mg of a compound according to Formula I, Formula II, and/or Formula III, or a pharmaceutically acceptable salt thereof, per day in single or multiple doses. In some embodiments, a compound or pharmaceutical composition according to any embodiment disclosed herein is administered daily for about 6 months. In some embodiments, a compound or pharmaceutical composition according to any embodiment disclosed herein is administered daily for at least about 6 months.

The terms “treat”, “treated”, or “treating” as used herein refer to both therapeutic treatment and prophylactic or preventative measures, wherein the object is to protect against (partially or wholly) or slow down (e.g., lessen or postpone the onset of) an undesired physiological condition, disorder or disease, or to obtain beneficial or desired clinical results such as partial or total restoration or inhibition in decline of a parameter, value, function or result that had or would become abnormal. For the purposes of this disclosure, beneficial or desired clinical results include, but are not limited to, alleviation of symptoms; diminishment of the extent or vigor or rate of development of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether or not it translates to immediate lessening of actual clinical symptoms, or enhancement or improvement of the condition, disorder or disease. Treatment seeks to elicit a clinically significant response without excessive levels of side effects. Treatment also includes prolonging survival as compared to expected survival if not receiving treatment.

The term “tissue” as used herein, generally refers to any aggregation of similarly specialized cells which are united in the performance of a particular function.

The term “cognitive decline” as used herein, refers to any negative change in an animal's cognitive function. For example, cognitive decline, includes but is not limited to, memory loss (e.g. behavioral memory loss), failure to acquire new memories, confusion, impaired judgment, personality changes, disorientation, or any combination thereof. A compound that is effective to treat cognitive decline can be thus effective by restoring long term neuronal potentiation (LTP) or long term neuronal depression (LTD) or a balance of synaptic plasticity measured electrophysiologically; inhibiting, treating, and/or abatement of neurodegeneration; inhibiting, treating, and/or abatement of general amyloidosis; inhibiting, treating, abatement of one or more of amyloid production, amyloid assembly, amyloid aggregation, and amyloid oligomer binding; inhibiting, treating, and/or abatement of a nonlethal effect of one or more of Abeta species on a neuron cell (such as synapse loss or dysfunction and abnormal membrane trafficking); and any combination thereof. Additionally, that compound can also be effective in treating Abeta related neurodegenerative diseases and disorders including, but not limited to dementia, including but not limited to Alzheimer's Disease (AD) including mild Alzheimer's disease, Down's syndrome, vascular dementia (cerebral amyloid angiopathy and stroke), dementia with Lewy bodies, HIV dementia, Mild Cognitive Impairment (MCI); Age-Associated Memory Impairment (AAMI); Age-Related Cognitive Decline (ARCD), preclinical Alzheimer's Disease (PCAD); and Cognitive Impairment No Dementia (CIND).

The term “Alzheimer's disease” as used herein refers to a type of dementia. Patients with Alzheimer's disease (AD) progress through a continuum of stages, starting with asymptomatic followed by mild cognitive impairment (MCI), mild dementia, moderate dementia, and severe dementia.

The term “brain penetrability” as used herein, refers to the ability of a drug, antibody or fragment, to cross the blood-brain barrier. In some embodiments, an animal pharmacokinetic (pK) study, for example, a mouse pharmacokinetic/blood-brain barrier study can be used to determine or predict brain penetrability. In some embodiments various concentrations of drug can be administered, for example at 3, 10 and 30 mg/kg, for example p.o. for 5 days and various pK properties are measured, e.g., in an animal model. In some embodiments, dose related plasma and brain levels are determined. In some embodiments, brain Cmax>100, 300, 600, 1000, 1300, 1600, or 1900 ng/mL. In some embodiments good brain penetrability is defined as a brain/plasma ratio of >0.1, >0.3, >0.5, >0.7, >0.8, >0.9, preferably >1, and more preferably >2, >5, or >10. In other embodiments, good brain penetrability is defined as greater than about 0.1%, 1%, 5%, greater than about 10%, and preferably greater than about 15% of an administered dose crossing the BBB after a predetermined period of time. In certain embodiments, the dose is administered orally (p.o.).

In other embodiments, the dose is administered intravenously (i.v.), prior to measuring pK properties.

The term “plasma stability” as used herein, refers to the degradation of compounds in plasma, for example, by enzymes such as hydrolases and esterases. Any of a variety of in vitro assays can be employed. Drugs are incubated in plasma over various time periods. The percent parent compound (analyte) remaining at each time point reflects plasma stability. Poor stability characteristics can tend to have low bioavailability. Good plasma stability can be defined as greater than 50% analyte remaining after 30 min, greater than 50% analyte remaining after 45 minutes, and preferably greater than 50% analyte remaining after 60 minutes.

The term “metabolic stability” as used herein, refers to the ability of the compound to survive first-pass metabolism (intestinal and hepatic degradation or conjugation of a drug administered orally). This can be assessed, for example, in vitro by exposure of the compounds to mouse or human hepatic microsomes. In some embodiments, good metabolic stability refers to a t_{1/2}>5 min, >10 min, >15 minutes, >20 minutes, and preferably >30 min upon exposure of a compound to mouse or human hepatic microsomes. In some embodiments, good metabolic stability refers to an Intrinsic Clearance Rate (Clᵢₙₜ) of <300 uL/min/mg, preferably <200 uL/min/mg, and more preferably <100 uL/min/mg.

Embodiments disclosed herein are directed to methods of treating Alzheimer's disease in a subject in need thereof, comprising administering to the subject a therapeutically effective amount of a compound, or a pharmaceutically acceptable salt thereof, according to Formula I:

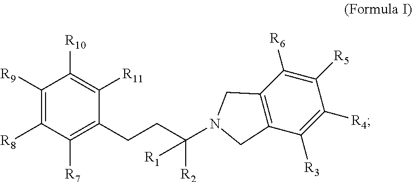

wherein: R₁ and R₂ are each independently selected from H, C₁-C₆ alkyl, or CH₂OR′; where R′ ═H or C₁-C₆ alkyl;
R₃, R₄, R₅, and R₆ are each independently selected from H, C₁-C₆ alkyl, OH, OCH₃, OCH(CH₃)₂, OCH₂CH(CH₃)₂, OC(CH₃)₃, O(C₁-C₆ alkyl), OCF₃, OCH₂CH₂OH, O(C₁-C₆ alkyl)OH, O(C₁-C₆ haloalkyl), F, Cl, Br, I, CF₃, CN, NO₂, NH₂, C₁-C₆ haloalkyl, C₁-C₆ hydroxyalkyl, C₁₋₆ alkoxy C₁₋₆alkyl, aryl, heteroaryl, C₃₋₇ cycloalkyl, heterocycloalkyl, alkylaryl, heteroaryl, CO₂R′, C(O)R′, NH(C₁₋₄ alkyl), N(C₁₋₄ alkyl)₂, NH(C₃₋₇ cycloalkyl), NHC(O)(C₁₋₄ alkyl), CONR′₂, NC(O)R′, NS(O)ₙR′, S(O)ₙNR′₂, S(O)ₙR′, C(O)O(C₁₋₄ alkyl), OC(O)N(R′)₂, C(O) (C₁₋₄ alkyl), and C(O)NH(C₁₋₄ alkyl); where n=0, 1, or 2; R′ are each independently H, CH₃, CH₂CH₃, C₃-C₆ alkyl, C₁-C₆ haloalkyl; or optionally substituted aryl, alkylaryl, piperazin-1-yl, piperidin-1-yl, morpholinyl, heterocycloalkyl, heteroaryl, C₁₋₆ alkoxy, NH(C₁₋₄ alkyl), or NH(C₁₋₄ alkyl)₂, wherein optionally substituted group is selected from C₁-C₆ alkyl or C₂-C₇ acyl;
or R₃ and R₄, together with the C atom to which they are attached form a form a 4-, 5-, 6- 7- or 8-membered cycloalkyl, aryl, heteroaryl, or heterocycloalkyl that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R³ and R⁴, or R⁴ and R⁵, are each independently selected from a bond, C, N, S, and O; or R₃ and R₄ are linked together to form a —O—C₁₋₂ methylene-O— group;
or R₄ and R₅, together with the C atom to which they are attached form a form a 4-, 5-, 6- 7- or 8-membered cycloalkyl, aryl, heteroaryl, or heterocycloalkyl that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R³ and R⁴, or R⁴ and R⁵, are each independently selected from a bond, C, N, S, and O; or R₄ and R₅ are linked together to form a —O—C₁₋₂ methylene-O— group;
R₇, R₈, R₉, R₁₀, and R₁₁ are each independently selected from H, C₁-C₆ alkyl, OH, OCH₃, OCH(CH₃)₂, OCH₂CH(CH₃)₂, OC(CH₃)₃, O(C₁-C₆ alkyl), OCF₃, OCH₂CH₂OH, O(C₁-C₆ alkyl)OH, O(C₁-C₆ haloalkyl), O(CO)R′, F, Cl, Br, I, CF₃, CN, NO₂, NH₂, C₁-C₆ haloalkyl, C₁-C₆ hydroxyalkyl, C₁₋₆ alkoxy C₁₋₆alkyl, aryl, heteroaryl, C₃₋₇ cycloalkyl, heterocycloalkyl, alkylaryl, heteroaryl, CO₂R′, C(O)R′, NH(C₁₋₄ alkyl), N(C₁₋₄ alkyl)₂, NH(C₃₋₇ cycloalkyl), NHC(O)(C₁₋₄ alkyl), CONR′₂, NC(O)R′, NS(O)ₙR′, S(O)ₙNR′₂, S(O)ₙR′, C(O)O(C₁₋₄ alkyl), OC(O)N(R′)₂, C(O) (C₁₋₄ alkyl), and C(O)NH(C₁₋₄ alkyl); where n=0, 1, or 2; R′ are each independently H, CH₃, CH₂CH₃, C₃-C₆ alkyl, C₁-C₆ haloalkyl, aryl, alkylaryl, piperazin-1-yl, piperidin-1-yl, morpholinyl, heterocycloalkyl, heteroaryl, C₁₋₆ alkoxy, NH(C₁₋₄ alkyl), or NH(C₁₋₄ alkyl)₂;
or R₇ and R₈, together with the N or C atoms to which they are attached form a form a 4-, 5-, 6- 7- or 8-membered cycloalkyl, aryl, heterocycloalkyl or heteroaryl group that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R⁹ and R¹⁰ are each independently selected from a bond, C, N, S, and O; or R₇ and R₈ are linked together to form a —O—C₁₋₂ methylene-O— group;
or R₈ and R₉, together with the N or C atoms to which they are attached form a form a 4-, 5-, 6- 7- or 8-membered cycloalkyl, aryl, heterocycloalkyl or heteroaryl group that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R⁹ and R¹⁰ are each independently selected from a bond, C, N, S, and O; or R₈ and R₉ are linked together to form a —O—C₁₋₂ methylene-O— group,
wherein each of the O, C₁₋₆ alkyl, C₁₋₆ haloalkyl, heteroaryl, aryl, heteroaryl, heterocycloalkyl, and cycloalkyl is optionally independently substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl and heterocycloalkyl; with the proviso that the following compounds are excluded:

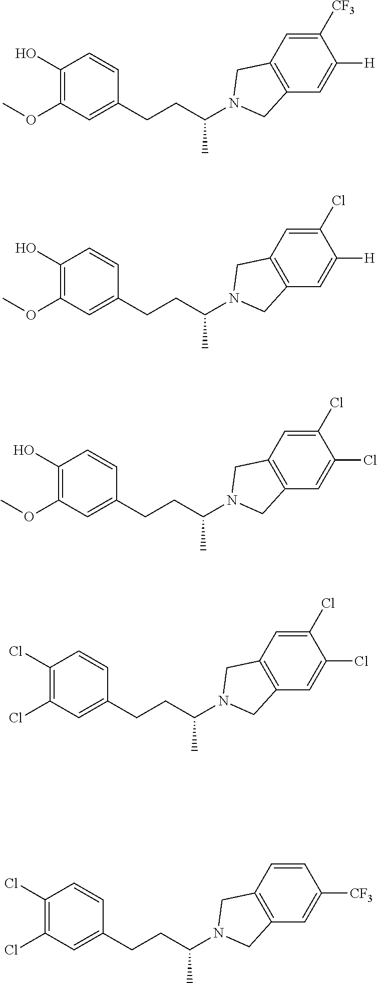 [SMILES: COc1cc(CC[C@@H](C)N2Cc3ccc(C(F)(F)F)cc3C2)ccc1O ; COc1cc(CC[C@@H](C)N2Cc3ccc(Cl)cc3C2)ccc1O ; COc1cc(CC[C@@H](C)N2Cc3cc(Cl)c(Cl)cc3C2)ccc1O ; C[C@H](CCc1ccc(Cl)c(Cl)c1)N1Cc2cc(Cl)c(Cl)cc2C1 ; C[C@H](CCc1ccc(Cl)c(Cl)c1)N1Cc2ccc(C(F)(F)F)cc2C1]

In some embodiments, excluded are certain compounds of the prior art. In some embodiments, the compounds described in Table 1 are disclosed in WO2013/029057 and/or WO2013/029060, each of which is incorporated by reference herein, and are disclaimed with respect to compositions or methods provided herein.

TABLE 1
. Disclaimed Compounds.

| Disclaimed Compound | Reference |
|---|---|
| 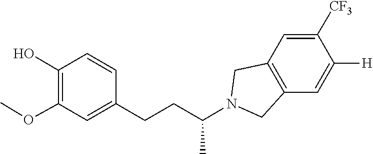 [SMILES: COc1cc(CC[C@@H](C)N2Cc3ccc(C(F)(F)F)cc3C2)ccc1O] | CogRx; Rishton, Catalano WO2013/029060, Table 1B, pp. 81-94; WO2013/029067, p. 48. |
| 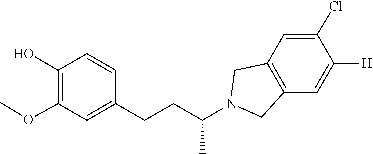 [SMILES: COc1cc(CC[C@@H](C)N2Cc3ccc(Cl)cc3C2)ccc1O] | CogRx; Rishton, Catalano WO2013/029060, Table 1B, pp. 81-94; WO2013/029067, p. 47. |
| 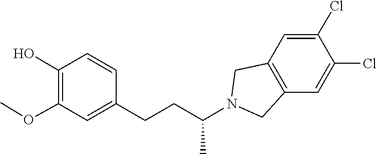 [SMILES: COc1cc(CC[C@@H](C)N2Cc3cc(Cl)c(Cl)cc3C2)ccc1O] | CogRx; Rishton, Catalano WO2013/029060, Table 1B, pp. 81-94; WO2013/029067, p. 48. |
| 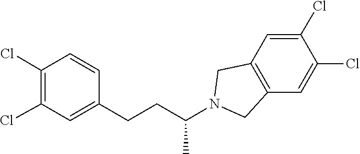 [SMILES: C[C@H](CCc1ccc(Cl)c(Cl)c1)N1Cc2cc(Cl)c(Cl)cc2C1] | CogRx; Rishton, Catalano WO2013/029060, Table 1B, pp. 81-94; WO2013/029067, p. 182. |
| 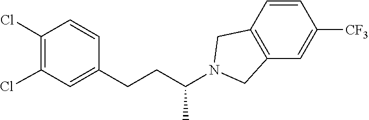 [SMILES: C[C@H](CCc1ccc(Cl)c(Cl)c1)N1Cc2ccc(C(F)(F)F)cc2C1] | CogRx; Rishton, Catalano WO2013/029060, Table 1B, pp. 81-94; WO2013/029067, pp. 55, 184. |

In some embodiments, the compound may comprise a racemic mixture or an enantiomer of compound of Formula I, wherein R₁, R₂, R₃, R₄, R₅, R₆, R₇, R₈, R₉, R₁₀, and R₁₁ are as described above.

In some embodiments, the compounds for use in the methods described herein may be a compound of Formula I:

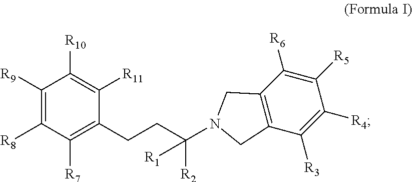

or a pharmaceutically acceptable salt thereof, wherein R₁, R₂, R₃, R₄, R₅, R₆, R₇, R₈, R₉, R₁₀, and R₁₁ are as defined herein, with the proviso that when R₁, R₃, R₆, R₇, R₁₀ and R₁₁ are each H; R₂ is CH₃; R₈ is OCH₃ or Cl; and R₉ is OH or Cl; then R₄ is not Cl or CF₃, and R₅ is not Cl or CF₃.

In other embodiments, the compounds for use in the methods described herein may be a compound of Formula I:

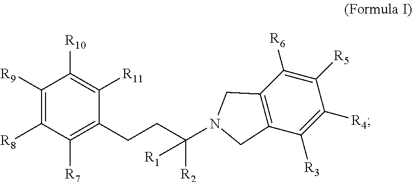

or a pharmaceutically acceptable salt thereof, wherein R₁, R₂, R₃, R₄, R₅, R₆, R₇, R₈, R₉, R₁₀, and R₁ are as defined herein, with the proviso that a compound according to Formula I wherein R₁, R₃, R₆, R₇, R₁₀ and R₁₁ are each H; R₂ is CH₃; R₈ is OCH₃ or Cl; and R₉ is OH or Cl; R₄ is Cl or CF₃, and R₅ is Cl or CF₃, is not a preferred compound.

In another embodiment, a pharmaceutical composition is provided for use in the methods described herein according to Formula I:

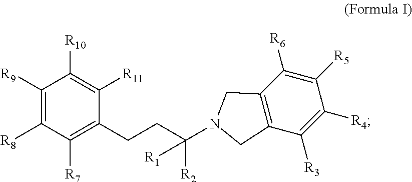

or a pharmaceutically acceptable salt thereof, and a pharmaceutically acceptable carrier, wherein R₁, R₂, R₃, R₄, R₅, R₆, R₇, R₈, R₉, R₁₀, and R₁₁ are as defined herein, with the proviso that when R₁, R₃, R₆, R₇, R₁₀ and R₁₁ are each H; R₂ is CH₃; R₈ is OCH₃ or Cl; and R₉ is OH or Cl; then R₄ is not Cl or CF₃, and R₅ is not Cl or CF₃.

In some embodiments, the compounds for use in the methods described herein may be a compound of Formula II:

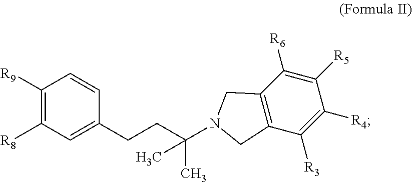

wherein R₃, R₄, R₅, R₆, R₈, and R₉ are as described herein.

In some embodiments, the compounds for use in the methods described herein may be a compound of Formula III, wherein R₃, R₄, R₅, R₆, R₇, R₈, R₉, R₁₀ and R₁₁ are as provided herein and wherein ----- are each independently selected from a single, double or triple bond.

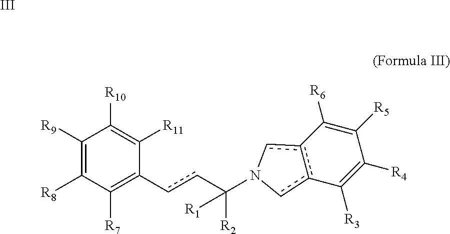

In some aspects, a compound according to Formula III is selected from:

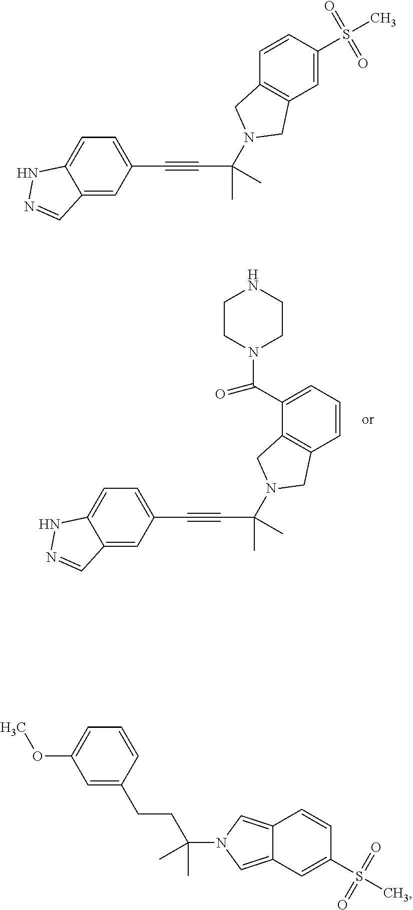

or a pharmaceutically acceptable salt thereof.

In some embodiments, the compounds for use in the methods described herein may comprise a racemic mixture or an enantiomer of a compound of Formula I, wherein R₃, R₄, R₅, R₆, R₈, and R₉ are as described herein.

In some embodiments, the compounds for use in the methods described herein may be a compound or a pharmaceutically acceptable salt of Formula I, wherein R₈ and R₉ are independently selected from OH, C₁₋₆ alkoxy, and hydroxy C₁₋₆ alkoxy.

In some embodiments, the compounds for use in the methods described herein may be a compound or a pharmaceutically acceptable salt of Formula I, wherein R₈ and R₉ are independently selected from OH and NH(C₁₋₄ alkyl).

In some embodiments, the compounds for use in the methods described herein may be a compound or a pharmaceutically acceptable salt of Formula I, wherein R₈ and R₉ are independently selected from H, halo, C₁₋₆ haloalkyl, and C₁₋₆ haloalkoxy.

In some embodiments, the compounds for use in the methods described herein may be a compound or a pharmaceutically acceptable salt of Formula I, wherein R₈ and R₉ are each independently selected from OH, halo, C₁₋₆ alkoxy and C₁₋₆ haloalkoxy and R₁ and R₂ are each independently C₁₋₆ alkyl.

In some embodiments, the compounds for use in the methods described herein may be a compound or a pharmaceutically acceptable salt of Formula I, wherein R₁ and R₂ are each methyl.

In some embodiments, the compounds for use in the methods described herein may be a compound or a pharmaceutically acceptable salt of Formula I, wherein one of R₁ and R₂ is methyl and the other is H.

In some embodiments, the compounds for use in the methods described herein may be a compound or a pharmaceutically acceptable salt of Formula I, wherein R₈ and R₉ are each independently selected from OH and C₁₋₆ alkoxy and R₁ and R₂ are each independently methyl.

In some embodiments, the compounds for use in the methods described herein may be a compound or a pharmaceutically acceptable salt of Formula I, wherein R₈ and R₉ are independently selected from H, halo, and C₁₋₆ haloalkyl, and R₁ and R₂ are each methyl.

In some embodiments, the compounds for use in the methods described herein may be a compound or a pharmaceutically acceptable salt of Formula I, wherein R⁸ and R⁹ are each independently selected from H, halo and C₁₋₆ haloalkyl.

In some embodiments, the compounds for use in the methods described herein may be a compound or a pharmaceutically acceptable salt of Formula I, wherein R₇ and R₁₁ are each H.

In some embodiments, the compounds for use in the methods described herein may be a compound or a pharmaceutically acceptable salt of Formula I, wherein R₃, R₄, R₅, and R₆ are each independently selected from H, halo, C₁₋₆ alkyl, C₁₋₆ haloalkyl and C₁₋₆ alkoxy.

In some embodiments, the compounds for use in the methods described herein may be a compound or a pharmaceutically acceptable salt of Formula I, wherein R₃, R₄ and R₅ are each independently selected from H, halo, C₁₋₆ alkyl, C₁₋₆ haloalkyl and C₁₋₆ alkoxy.

In some embodiments, the compounds for use in the methods described herein may be a compound or a pharmaceutically acceptable salt of Formula I, wherein R₃, R₄, R₅, and R₆ are each independently selected from H, halo, S(O)₁R′, C(O)OR′, C(O)N(R′)₂, and C(O)R′; where n=2; R′ are each independently H, CH₃, CH₂CH₃, C₃-C₆ alkyl, C₁-C₆ haloalkyl, or optionally C₁-C₆ alkyl or C₂-C₇ acyl substituted aryl, alkylaryl, piperazinyl, piperidinyl, morpholinyl, heterocycloalkyl, and heteroaryl.

In some embodiments, the compounds for use in the methods described herein may be a compound or a pharmaceutically acceptable salt of Formula I, wherein R₃, R₄ and R₅ are each independently selected from H, halo, S(O)ₙR′, and C(O)R′; where n=2; R′ are each independently CH₃, CH₂CH₃, C₃-C₆ alkyl, aryl, piperazin-1-yl, piperidin-1-yl, and morpholinyl-4-yl.

In some embodiments, the compounds for use in the methods described herein may be a compound or a pharmaceutically acceptable salt of Formula I, wherein R₃, R₄ and R₅ are each independently selected from H, halo, S(O)ₙR′, and C(O)R′; where n=2; R′ are each independently CH₃, CH₂CH₃, C₃-C₆ alkyl, aryl, piperazin-1-yl, piperidin-1-yl, and morpholinyl-4-yl; R₈ and R₉ are each independently selected from OH, halo, C₁₋₆ alkoxy and C₁₋₆ haloalkoxy; and R₁ and R₂ are each methyl.

In some embodiments, the compounds for use in the methods described herein may be a compound or a pharmaceutically acceptable salt of Formula I, wherein R₃ and R₄ or R₄ and R₅ together with the C atom to which they are attached form a 6-membered cycloalkyl, or a heterocycloalkyl, aryl or heteroaryl ring.

In some embodiments, the compounds for use in the methods described herein may be a compound or a pharmaceutically acceptable salt of Formula I, wherein R₃ and R₄ or R₄ and R₅ are O, and are linked together to form a —O—C₁₋₂ methylene-O— group.

In some embodiments, the compounds for use in the methods described herein may be a compound or a pharmaceutically acceptable salt of Formula I, wherein R² and R³ are independently selected from H, OH, halo, C₁₋₆ alkoxy and C₁₋₆ haloalkyl.

In some embodiments, the compounds for use in the methods described herein may be a compound or a pharmaceutically acceptable salt of Formula II, wherein R₃ and R₄ are independently selected from H, Cl, F, —OMe, —CF₃, S(O)ₙR′, and C(O)R′; where n=2; R′ are each independently H, CH₃, CH₂CH₃, C₃-C₆ alkyl, aryl, piperazin-1-yl, piperidin-1-yl, and morpholinyl-4-yl; R₈ and R₉ are each independently selected from OH and C₁₋₆ alkoxy.

In some embodiments, the compounds for use in the methods described herein may be a compound or a pharmaceutically acceptable salt of Formula I, wherein R² and R³ are independently selected from H, OH, Cl, F, —OMe, and —CF₃, wherein R⁷ and R⁸ are each independently selected from H and C₁₋₆ alkyl, wherein R⁹ is H, and wherein R⁵ and R⁶ are each independently selected from H and C₁₋₆ haloalkyl.

Preferred salts for use in the disclosure include the hydrochloride and fumarate salts of the above compounds.

These have been synthesized in accordance with general methods provided herein and specific synthetic examples with any additional steps being well within the skill in the art.

Several of these compounds have been tested in various assays as detailed herein and have been found active. Tested compounds also display increased bioavailability by reference to compounds disclosed in WO 2010/110855.

In some embodiments, each of the general formulae above may contain a proviso to remove one or more of the following compounds:

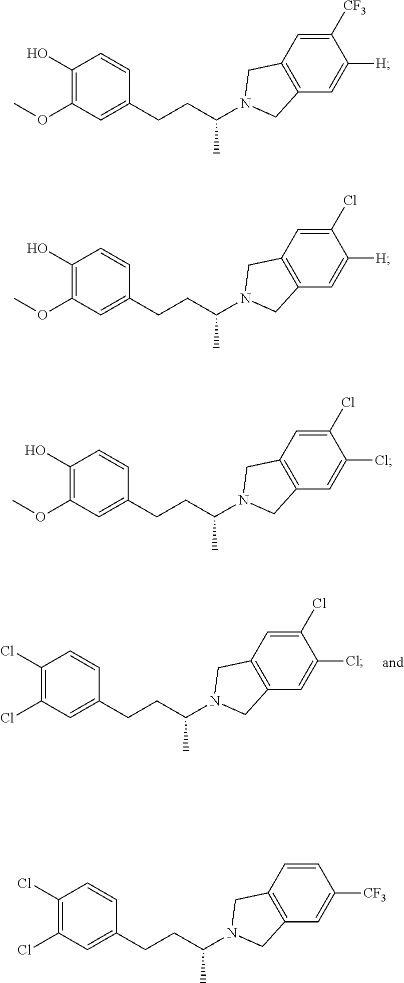

Compounds according to Formula I, Formula II, and/or Formula III have been synthesized in accordance with general methods provided in WO 2015/116923, incorporated herein by reference. Several of these compounds have been tested in various assays as detailed herein and have been found active. Tested compounds also display increased bioavailability by reference to compounds disclosed in WO 2010/110855, incorporated herein by reference.

In some embodiments, the compound of Formula I is

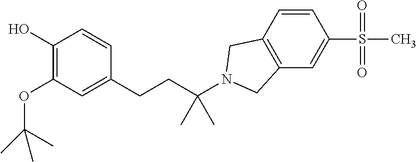 [SMILES: CC(C)(C)Oc1cc(CCC(C)(C)N2Cc3ccc(S(C)(=O)=O)cc3C2)ccc1O]

or a pharmaceutically acceptable salt thereof. This compound is also known as CT1812 and is referred to by this name in the examples. In some embodiments, the pharmaceutically acceptable salt is the fumarate salt.

In some embodiments, the therapeutically effective amount of any of the compounds disclosed herein is from about 0.0001 mg to about 2000 mg, about 0.0001 mg to about 1500 mg, about 0.0001 mg to about 1200 mg, about 0.0001 mg to about 1000 mg, about 0.0001 mg to about 800 mg, about 0.0001 mg to about 500 mg, about 0.0001 mg to about 250 mg, about 0.0001 mg to about 200 mg, or about 0.0001 mg to about 100 mg. In some embodiments, the therapeutically effective amount of the compound of Formula I is about 1 mg, about 5 mg, about 10 mg, about 25 mg, about 30 mg, about 50 mg, about 90 mg, about 180 mg, about 280 mg, about 450 mg, about 560 mg, about 840 mg, about 1120 mg, about 1500 mg, about 2000 mg.

### Salts, Solvates, Stereoisomers, Derivatives, Prodrugs and Active Metabolites of the Compounds for Use in the Methods Described Herein.

The disclosure further encompasses salts, solvates, stereoisomers, prodrugs and active metabolites of the compounds of any of the formulae above.

The acid addition salts of the compounds of any of the formulae above may be prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the disclosure.

Also included are both total and partial salts, that is to say salts with 1, 2 or 3, preferably 2, equivalents of base per mole of acid of a, e.g., formula I compound or salt, with 1, 2 or 3 equivalents, preferably 1 equivalent, of acid per mole of base of a any of the formulae above compound.

For the purposes of isolation or purification it is also possible to use pharmaceutically unacceptable salts. However, only the pharmaceutically acceptable, non-toxic salts are used therapeutically and they are therefore preferred.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine.

The base addition salts of said acidic compounds are prepared by contacting the free acid form with a sufficient amount of the desired base to produce the salt in the conventional manner. The free acid form may be regenerated by contacting the salt form with an acid and isolating the free acid.

Compounds of the disclosure may have both a basic and an acidic center and may therefore be in the form of zwitterions or internal salts.

Typically, a pharmaceutically acceptable salt of a compound of any of the formulae above may be readily prepared by using a desired acid or base as appropriate. The salt may precipitate from solution and be collected by filtration or may be recovered by evaporation of the solvent. For example, an aqueous solution of an acid such as hydrochloric acid may be added to an aqueous suspension of a compound of any of the formulae above and the resulting mixture evaporated to dryness (lyophilized) to obtain the acid addition salt as a solid.

Alternatively, a compound of any of the formulae above may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compound of the disclosure are within the scope of the disclosure. The salts of the compound of any of the formulae above may form solvates (e.g., hydrates) and the disclosure also includes all such solvates. The meaning of the word “solvates” is well known to those skilled in the art as a compound formed by interaction of a solvent and a solute (i.e., solvation). Techniques for the preparation of solvates are well established in the art (see, for example, Brittain. Polymorphism in Pharmaceutical solids. Marcel Decker, New York, 1999.).

The disclosure also encompasses N-oxides of the compounds of formulas I. The term “N-oxide” means that for heterocycles containing an otherwise unsubstituted sp² N atom, the N atom may bear a covalently bound O atom, i.e., —N→O. Examples of such N-oxide substituted heterocycles include pyridyl N-oxides, pyrimidyl N-oxides, pyrazinyl N-oxides and pyrazolyl N-oxides.

Compounds of any of the formulae above may have one or more chiral centers and, depending on the nature of individual substituents, they can also have geometrical isomers. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has a chiral center, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomer respectively). A chiral compound can exist as either an individual enantiomer or as a mixture of enantiomers. A mixture containing equal proportions of the enantiomers is called a “racemic mixture”. A mixture containing unequal portions of the enantiomers is described as having an “enantiomeric excess” (ee) of either the R or S compound. The excess of one enantiomer in a mixture is often described with a % enantiomeric excess (% ee) value determined by the formula:

$$\% ee = ( R ) - ( S ) / ( R ) + ( S )$$

The ratio of enantiomers can also be defined by “optical purity” wherein the degree at which the mixture of enantiomers rotates plane polarized light is compared to the individual optically pure R and S compounds. Optical purity can be determined using the following formula:

$$Optical purity = {enant}_{. major} / ( {enant}_{. major} + {enant}_{. minor} )$$

Compounds of any of the formulae above can also be a substantially pure (+) or (−) enantiomer of the compounds described herein. In some embodiments, a composition comprising a substantially pure enantiomer comprises at least 90, 91, 92, 93, 94, 95, 96, 97, 98, or 99% of one enantiomer. In some embodiments, a composition comprising a substantially pure enantiomer is at least 99.5% one enantiomer. In some embodiments, the composition comprises only one enantiomer of a compound described herein.

The disclosure encompasses all individual isomers of the compounds of any of the formulae above. The description or naming of a particular compound in the specification and claims is intended to include both individual enantiomers and mixtures, racemic or otherwise, thereof. Methods for the determination of stereochemistry and the resolution or stereotactic synthesis of stereoisomers are well-known in the art. Specifically, there is a chiral center shown in the compounds of any of the formulae above which gives rise to one set of enantiomers. Additional chiral centers may be present depending on the substituents.

For many applications, it is preferred to carry out stereoselective syntheses and/or to subject the reaction product to appropriate purification steps so as to produce substantially optically pure materials. Suitable stereoselective synthetic procedures for producing optically pure materials are well known in the art, as are procedures for purifying racemic mixtures into optically pure fractions. Those of skill in the art will further recognize that disclosure compounds may exist in polymorphic forms wherein a compound is capable of crystallizing in different forms. Suitable methods for identifying and separating polymorphisms are known in the art.

Diastereomers differ in both physical properties and chemical reactivity. A mixture of diastereomers can be separated into enantiomeric pairs based on solubility, fractional crystallization or chromatographic properties, e.g., thin layer chromatography, column chromatography or HPLC.

Purification of complex mixtures of diastereomers into enantiomers typically requires two steps. In a first step, the mixture of diastereomers is resolved into enantiomeric pairs, as described above. In a second step, enantiomeric pairs are further purified into compositions enriched for one or the other enantiomer or, more preferably resolved into compositions comprising pure enantiomers. Resolution of enantiomers typically requires reaction or molecular interaction with a chiral agent, e.g., solvent or column matrix. Resolution may be achieved, for example, by converting the mixture of enantiomers, e.g., a racemic mixture, into a mixture of diastereomers by reaction with a pure enantiomer of a second agent, i.e., a resolving agent. The two resulting diastereomeric products can then be separated. The separated diastereomers are then reconverted to the pure enantiomers by reversing the initial chemical transformation.

Resolution of enantiomers can also be accomplished by differences in their non-covalent binding to a chiral substance, e.g., by chromatography on homochiral adsorbants. The noncovalent binding between enantiomers and the chromatographic adsorbant establishes diastereomeric complexes, leading to differential partitioning in the mobile and bound states in the chromatographic system. The two enantiomers therefore move through the chromatographic system, e.g., column, at different rates, allowing for their separation.

Chiral resolving columns are well known in the art and are commercially available (e.g., from MetaChem Technologies Inc., a division of ANSYS Technologies, Inc., Lake Forest, CA). Enantiomers can be analyzed and purified using, for example, chiral stationary phases (CSPs) for HPLC. Chiral HPLC columns typically contain one form of an enantiomeric compound immobilized to the surface of a silica packing material.

D-phenylglycine and L-leucine are examples of Type I CSPs and use combinations of π-π interactions, hydrogen bonds, dipole-dipole interactions, and steric interactions to achieve chiral recognition. To be resolved on a Type I column, analyte enantiomers must contain functionality complementary to that of the CSP so that the analyte undergoes essential interactions with the CSP. The sample should preferably contain one of the following functional groups: π-acid or π-base, hydrogen bond donor and/or acceptor, or an amide dipole. Derivatization is sometimes used to add the interactive sites to those compounds lacking them. The most common derivatives involve the formation of amides from amines and carboxylic acids.

The MetaChiral ODM™ is an example of a type II CSP. The primary mechanisms for the formation of solute-CSP complexes is through attractive interactions, but inclusion complexes also play an important role. Hydrogen bonding, π-π interactions, and dipole stacking are important for chiral resolution on the MetaChiral™ ODM. Derivatization may be necessary when the solute molecule does not contain the groups required for solute-column interactions. Derivatization, usually to benzylamides, may be required for some strongly polar molecules like amines and carboxylic acids, which would otherwise interact strongly with the stationary phase through non-specific-stereo interactions.

Where applicable, compounds of any of the formulae above can be separated into diastereomeric pairs by, for example, separation by column chromatography or TLC on silica gel. These diastereomeric pairs are referred to herein as diastereomer with upper TLC Rf; and diastereomer with lower TLC Rf. The diastereomers can further be enriched for a particular enantiomer or resolved into a single enantiomer using methods well known in the art, such as those described herein.

The relative configuration of the diastereomeric pairs can be deduced by the application of theoretical models or rules (e.g. Cram's rule, the Felkin-Ahn model) or using more reliable three-dimensional models generated by computational chemistry programs. In many instances, these methods are able to predict which diastereomer is the energetically favored product of a chemical transformation. As an alternative, the relative configuration of the diastereomeric pairs can be indirectly determined by discovering the absolute configurations of a single enantiomer in one (or both) of the diastereomeric pair(s).

The absolute configuration of the stereocenters can be determined by very well known method to those skilled in the art (e.g. X-Ray diffraction, circular dichroism). Determination of the absolute configuration can be useful also to confirm the predictability of theoretical models and can be helpful to extend the use of these models to similar molecules prepared by reactions with analogous mechanisms (e.g. ketone reductions and reductive amination of ketones by hydrides).

The disclosure may also encompass stereoisomers of the Z-E type, and mixtures thereof due to R₂-R₃ substituents to the double bond not directly linked to the ring. Additional Z-E stereoisomers are encountered when m is not 1 and m and n are different. The Cahn-Ingold-Prelog priority rules are applied to determine whether the stereoisomers due to the respective position in the plane of the double bond of the doubly bonded substituents are Z or E. The stereoisomer is designated as Z (zusammen=together) if the 2 groups of highest priority lie on the same side of a reference plane passing through the C═C bond. The other stereoisomer is designated as E (entgegen=opposite).

Mixture of stereoisomers of E-Z type can be separated (and/or characterized) in their components using classical method of purification that are based on the different chemico-physical properties of these compounds. Included in these method are fractional crystallization, chromatography carried out by low, medium or high pressure techniques, fractional distillation and any other method very well known to those skilled in the art.

The disclosure also encompasses prodrugs of the compounds of any of the formulae above, i.e., compounds which release an active drug according to any of the formulae above in vivo when administered to a mammalian subject. A prodrug is a pharmacologically active or more typically an inactive compound that is converted into a pharmacologically active agent by a metabolic transformation. Prodrugs of a compound of any of the formulae above are prepared by modifying functional groups present in the compound of any of the formulae above in such a way that the modifications may be cleaved in vivo to release the parent compound. In vivo, a prodrug readily undergoes chemical changes under physiological conditions (e.g., are hydrolyzed or acted on by naturally occurring enzyme(s)) resulting in liberation of the pharmacologically active agent. Prodrugs include compounds of any of the formulae above wherein a hydroxy, amino, or carboxy group is bonded to any group that may be cleaved in vivo to regenerate the free hydroxyl, amino or carboxy group, respectively. Examples of prodrugs include, but are not limited to esters (e.g., acetate, formate, and benzoate derivatives) of compounds of any of the formulae above or any other derivative which upon being brought to the physiological pH or through enzyme action is converted to the active parent drug. Conventional procedures for the selection and preparation of suitable prodrug derivatives are described in the art (see, for example, Bundgaard. Design of Prodrugs. Elsevier, 1985).

Prodrugs may be administered in the same manner as the active ingredient to which they convert or they may be delivered in a reservoir form, e.g., a transdermal patch or other reservoir which is adapted to permit (by provision of an enzyme or other appropriate reagent) conversion of a prodrug to the active ingredient slowly over time, and delivery of the active ingredient to the patient.

The disclosure also encompasses metabolites.

### Sigma-2 Receptor Ligands for Selection as Sigma-2 Receptor Modulators

The isoindoline compounds provided herein act as high affinity, selective sigma-2 functional modulators or selective sigma-2 functional antagonists having the therapeutic phenotype, and good drug-like properties, and thus can be used to treat Abeta oligomer-induced synaptic dysfunction.

In certain embodiments, the compositions are provided comprising isoindoline compounds of Formula I, Formula II, or Formula III as selective sigma-2 functional modulators that have high binding affinity to the sigma receptors. In some embodiments, the sigma receptors include both the sigma-1 and sigma-2 subtypes. See Hellewell, S. B. and Bowen, W. D., Brain Res. 527: 224-253 (1990); and Wu, X.-Z. et al., J. Pharmacol. Exp. Ther. 257: 351-359 (1991). A sigma receptor binding assay which quantitates the binding affinity of a putative ligand for both sigma sites (against ³H-DTG, which labels both sites with about equal affinity) is disclosed by Weber et al., Proc. Natl. Acad. Sci (USA) 83: 8784-8788 (1986). Alternatively, [³H]pentozocine may be used to selectively label the sigma-1 binding site in a binding assay. A mixture of [³H]DTG and unlabeled (+)pentazocine is used to selectively label the sigma-2 site in a binding assay. The disclosure is also directed to compositions comprising certain ligands which are selective for the sigma-1 and sigma-2 receptors and act as sigma-2 functional modulators or sigma-2 functional antagonists as well as use of these compositions to treat Abeta oligomer-induced synaptic dysfunction. The discovery of such ligands which are selective for one of the two sigma receptor subtypes may be an important factor in identifying compounds which are efficacious in treating central nervous system disorders with minimal side effects.

In some embodiments, isoindoline compounds of Formula I, Formula II, or Formula III exhibit sigma-2 antagonist activity, high affinity for the sigma-2 receptor, and the ability to block soluble Abeta oligomer binding or Abeta oligomer-induced synaptic dysfunction.

In some embodiments, the sigma-2 modulators, are designed to enhance their ability to cross the blood-brain barrier.

In some embodiments, the specific sigma-2 receptor modulator compound blocks binding between soluble Abeta oligomers and a sigma-2 receptor.

In some embodiments, the sigma-2 modulator compound exhibits high affinity for the sigma-2 receptor.

In some embodiments, sigma-2 receptor modulators for use in the present disclosure are selected from among sigma-2 receptor ligand compounds that also meet additional selection criteria. Additional criteria are used to select sigma-2 receptor modulators for use in the present disclosure from among sigma-2 receptor ligands. Additional selection criteria include: acting as a functional antagonist in a neuronal cell with respect to inhibiting soluble Abeta oligomer induced synapse loss, and inhibiting soluble Abeta oligomer induced deficits in a membrane trafficking assay; having high selectivity for one or more sigma receptors compared to any other non-sigma receptor; exhibiting high affinity at a sigma-2 receptor; and exhibiting good drug-like properties including good brain penetrability, good metabolic stability and good plasma stability. In some embodiments, the sigma-2 receptor modulator is further selected on the basis of exhibiting one or more of the additional following properties: does not affect trafficking or synapse number in the absence of Abeta oligomer; does not induce caspase-3 activity in a neuronal cell; inhibits induction of caspase-3 activity by a sigma-2 receptor agonist; and/or decreases or protects against neuronal toxicity in a neuronal cell caused by a sigma-2 receptor agonist.

In some embodiments, certain sigma-2 receptor ligand compounds subject to further selection criteria are selected from compounds described herein and can be synthesized according to the methods described herein or in WO 2011/014880 (Application No. PCT/US2010/044136), WO 2010/118055 (Application No. PCT/US2010/030130), WO 2011/0106785 (Application No. PCT/US2011/026530), WO 2012/106426 (Application No. PCT/US2012/023483), WO 2013/029057 (Application No. PCT/US2012/052572), and WO 2013/029060 (Application No. PCT/US2012/052578), each of which is incorporated herein by reference in its entirety.

In some embodiments, the compounds for use in the methods described herein have an IC₅₀ value of less than 100 μM, 50 μM, 20 μM, 15 μM, 10 μM, 5 M, 1 μM, 500 nM. 100 nM, 50 nM, or 10 nM with respect to inhibition of one or more of the effect of Abeta oligomers on neurons (such as neurons in the brain), amyloid assembly or disruption thereof, and amyloid (including amyloid oligomer) binding, and amyloid deposition. In some embodiments, the compound has an IC₅₀ value of less than 100 μM, 50 μM, 20 μM, 15 μM, 10 μM, 5 μM, 1 μM, 500 nM, 100 nM, 50 nM, or 10 nM with respect to inhibition of the activity/effect of Abeta species such as oligomers on neurons (such as central nervous system neurons).

In some embodiments, percentage inhibition by the compounds for use in the methods described herein of one or more of the effects of Abeta species such as oligomers on neurons (such as neurons in the brain), such as amyloid (including amyloid oligomer) binding to synapses, and abnormalities in membrane trafficking mediated by Abeta oligomer was measured at a concentration of from 10 nM to 10 μM. In some embodiments, the percentage inhibition measured is about 1% to about 20%, about 20% to about 50%, about 1% to about 50%, or about 1% to about 80%. Inhibition can be assessed for example by quantifying synapse number of a neuron prior to and after exposure to an amyloid beta species or quantifying the number of synapses in the presence of both of a sigma-2 modulator and the Abeta species wherein the sigma-2 modulator is simultaneous with, or precedes or follows, Abeta species exposure. As another example, inhibition can be assessed by determining membrane trafficking and comparing one or more parameters that measure exocytosis rate and extent, endocytosis rate and extent, or other indicators of cell metabolism in the presence and absence of an Abeta species and in the presence and absence of a sigma-2 modulator according to the disclosure. The present inventors have adduced biochemical assay evidence that compounds of the disclosure also inhibit amyloid aggregation (data not shown).

In some embodiments, the compounds for use in the methods described herein bind specifically to a sigma-2 receptor. A compound that binds specifically to a specific receptor refers to a compound that has a preference for one receptor over another. For example, although a compound may be capable of binding both sigma-1 and sigma-2 receptor, a compound can be said to be specific for a sigma-2 receptor when it binds with a binding affinity that is at least 10% greater than to the sigma-1 receptor. In some embodiments, the specificity is at least 10, 20, 30, 40, 50, 60, 70, 80, 90, 100, 200, 300, 400, 500, or 1000% greater for one binding partner (e.g. receptor) than a second binding partner.

In determining whether a compound of any of the formulae above and other compounds described as sigma-2 antagonists above is effective in treating the various conditions described herein, in vitro assays can be used. The in vitro assays have been correlated with an in vivo effect. For example, in the in vitro assays described herein, it can also be used in vivo to treat or ameliorate the conditions described herein including inhibiting or restoring synapse loss, modulating a membrane trafficking change in neuronal cells, protecting against or restoring memory loss, and treating cognitive decline conditions, diseases and disorders such as MCI and Alzheimer's disease. The assays are based, in part, on the amyloid beta oligomers and their function in binding to neurons at the synapses and the effect that amyloid beta oligomers have on neurons in vitro. In some embodiments, an Abeta oligomer receptor in neurons which the present inventors believe includes a sigma-2 protein is contacted with an amyloid beta assembly as described herein and a compound according to Formula I, II, or III that binds to the sigma-2 protein will inhibit the binding of the amyloid beta assembly to the receptor. In competitive radioligand binding assays the present inventors have shown that the present compounds are specific for the sigma-2 receptor. The inventors have also shown that the compounds of the disclosure inhibit binding of Abeta oligomers to their heretofore unidentified receptor on the surface of neurons. In some embodiments, methods are provided to determine a compound of any above formula's sigma-2 ligand efficacy in neuronal signaling. In some embodiments, the method comprises contacting a cell, such as but not limited to, a primary neuron, with a sigma-2 ligand and measuring neuronal function. In some embodiments, the cell is contacted in vitro. In some embodiments the cell is contacted in vivo. The neuronal activity can be signaling activity, electrical activity, the production or release of synaptic proteins, and the like. A sigma-2 modulator that enhances or restores the signaling is identified as a compound that is effective in modulating neuronal activity. In some embodiments, the cell is derived from a pathological sample. In some embodiments, the cell is derived from a subject having a neurodegenerative disease. In some embodiments, the neurodegenerative disease is MCI or Alzheimer's Disease, especially mild Alzheimer's disease.

### Pharmaceutical Compositions Comprising a Sigma-2 Receptor Modulator

The compounds provided herein can be administered in the form of pharmaceutical compositions. These compositions can be prepared in a manner well known in the pharmaceutical art, and can be administered by a variety of routes, depending upon whether local or systemic treatment is desired and upon the area to be treated.

Thus, another embodiment of the disclosure comprises pharmaceutical compositions for use in the methods described herein comprising a pharmaceutically acceptable excipient or diluent and a therapeutically effective amount of a compound of the disclosure, including an enantiomer, diastereomer, N-oxide or pharmaceutically acceptable salt thereof.

While it is possible that a compound may be administered as the bulk substance, it is preferable to present the active ingredient in a pharmaceutical formulation, e.g., wherein the active agent is in admixture with a pharmaceutically acceptable carrier selected with regard to the intended route of administration and standard pharmaceutical practice.

Accordingly, in one aspect, the disclosure provides a pharmaceutical composition comprising at least one compound, antibody or fragment, of any of the formulae above and other compounds described as sigma-2 receptor modulators above described above or a pharmaceutically acceptable derivative (e.g., a salt or solvate) thereof, and, optionally, a pharmaceutically acceptable carrier. In particular, the disclosure provides a pharmaceutical composition comprising a therapeutically effective amount of at least one compound of any of the formulae above or a pharmaceutically acceptable derivative thereof, and, optionally, a pharmaceutically acceptable carrier.

### Combinations

For the compositions and methods of the disclosure, a compound of any of the formulae above and other compounds described as sigma-2 receptor modulators above described above may be used in combination with other therapies and/or active agents.

In some embodiments, the compounds for use in the methods described herein can be combined with one or more of a cholinesterase inhibitor, an N-methyl-D-aspartate (NMDA) glutamate receptor antagonist, a beta-amyloid specific antibody, a beta-secretase 1 (BACE1, beta-site amyloid precursor protein cleaving enzyme 1) inhibitor, a tumor necrosis factor alpha (TNF alpha) modulator, an intravenous immunoglobulin (IVIG), or a prion protein antagonist.

In some embodiments the sigma-2 receptor modulator is combined with a cholinesterase inhibitor selected from tacrine (COGNEX®; Sciele), donepezil (ARICEPT®; Pfizer), rivastigmine (EXELON®; Novartis), or galantamine (RAZADYNE®; Ortho-McNeil-Janssen). In some embodiments, the sigma-2 receptor modulator is combined with a TNFalpha modulator that is perispinal etanercept (ENBREL®, Amgen/Pfizer). In some embodiments, the sigma-2 receptor modulator is combined with a beta-amyloid specific antibody selected from bapineuzumab (Pfizer), solanezumab (Lilly), PF-04360365 (Pfizer), GSK933776 (GlaxoSmithKline), Gammagard (Baxter) or Octagam (Octapharma). In some embodiments, the sigma-2 receptor modulator is combined with an NMDA receptor antagonist that is memantine (NAMENDAR; Forest). In some embodiments, the BACE1 inhibitor is MK-8931 (Merck). In some embodiments, the sigma-2 receptor modulator is combined with IVIG as described in Magga et al., J Neuroinflam 2010, 7:90, Human intravenous immunoglobulin provides protection against Ab toxicity by multiple mechanisms in a mouse model of Alzheimer's disease, and Whaley et al., 2011, Human Vaccines 7:3, 349-356, Emerging antibody products and Nicotiana manufacturing; each of which is incorporated herein by reference. In some embodiments, the sigma-2 receptor modulator is combined with a prion protein antagonist as disclosed in Strittmatter et al., US 2010/0291090, which is incorporated herein by reference.

Accordingly, the disclosure provides, in a further aspect, pharmaceutical compositions comprising at least one compound of any of the formulae above or a pharmaceutically acceptable derivative thereof, a second active agent, and optionally, a pharmaceutically acceptable carrier.

When combined in the same formulation it will be appreciated that the two or more compounds must be stable and compatible with each other and the other components of the formulation. When formulated separately they may be provided in any convenient formulation, conveniently in such manner as are known for such compounds in the art.

Preservatives, stabilizers, dyes and even flavoring agents may be provided in the pharmaceutical composition. Examples of preservatives include sodium benzoate, ascorbic acid and esters of p-hydroxybenzoic acid. Antioxidants and suspending agents may be also used.

With respect to combinations including biologics such as monoclonal antibodies or fragments, suitable excipients will be employed to prevent aggregation and stabilize the antibody or fragment in solution with low endotoxin, generally for parenteral, for example, intravenous, administration. For example, see Formulation and Delivery Issues for Monoclonal Antibody Therapeutics, Daugherty et al., in Current Trends in Monoclonal Antibody Development and Manufacturing, Part 4, 2010, Springer, New York pp 103-129.

The compounds of the disclosure may be milled using known milling procedures such as wet milling to obtain a particle size appropriate for tablet formation and for other formulation types. Finely divided (nanoparticulate) preparations of the compounds of the disclosure may be prepared by processes known in the art, for example see WO 02/00196 (SmithKline Beecham).

### Routes of Administration and Unit Dosage Forms

In some embodiments, the therapeutically effective amount of the compounds of Formula I, Formula II, and Formula III is from about 0.0001 mg to about 2000 mg, about 0.0001 mg to about 1500 mg, about 0.0001 mg to about 1200 mg, about 0.0001 mg to about 1000 mg, about 0.0001 mg to about 800 mg, about 0.0001 mg to about 500 mg, about 0.0001 mg to about 250 mg, about 0.0001 mg to about 200 mg, or about 0.0001 mg to about 100 mg. In some embodiments, the therapeutically effective amount of the compounds Formula I-II is about 100 mg to about 300 mg. In some embodiments, the therapeutically effective amount of the compounds of Formula I-111 is about 1 mg, about 5 mg, about 10 mg, about 25 mg, about 30 mg, about 50 mg, about 90 mg, about 100 mg, about 180 mg, about 280 mg, about 300 mg, about 450 mg, about 560 mg, about 840 mg, about 1120 mg, about 1500 mg, about 2000 mg.

In some embodiments, the compounds disclosed herein can be administered once daily (QD), twice daily, once in two days, once in three days, once in four days, once in five days, once in six days, or once in seven days. In some embodiments, the compounds disclosed herein can be administered for at least about 6 months. In some embodiments, the compounds disclosed herein can be administered for at least about 12 months. In some embodiments, the compounds disclosed herein can be administered once daily (QD) for 2 consecutive days, for 3 consecutive days, for 4 consecutive days, for 5 consecutive days, for 6 consecutive days, for 7 consecutive days, for 8 consecutive days, for 9 consecutive days, for 10 consecutive days, or for 14 consecutive days. A dosing cycle may include administration for about 1 week, about 2 weeks, about 3 weeks, about 4 weeks, about 5 weeks, about 6 weeks, about 7 weeks, about 8 weeks, about 9 weeks, or about 10 weeks. After this cycle, a subsequent cycle may begin approximately 1, 2, 3, 4, 5, 6, 7, 8, 9, 10, 11, or 12 weeks later. The treatment regime may include 1, 2, 3, 4, 5, or 6 cycles, each cycle being spaced apart by approximately 1, 2, 3, 4, 5, 6, 7, 8, 9, 10, 11, or 12 weeks. In some embodiments, the dosing can be in fed state or in fasting state.

In some embodiments, the dosing of the compounds of Formula I, Formula II, and Formula III is such that they can achieve a Cmax of about 1 ng/mL to about 2000 ng/mL, about 1 ng/mL to about 1500 ng/mL, about 1 ng/mL to about 1000 ng/mL, about 1 ng/mL to about 750 ng/mL, about 1 ng/mL to about 500 ng/mL, about 1 ng/mL to about 200 ng/mL, about 1 ng/mL to about 100 ng/mL, about 1 ng/mL to about 50 ng/mL, or about 1 ng/mL to about 10 ng/mL. Specific examples include about 1 ng/mL, about 5 ng/mL, about 20 ng/mL, about 80 ng/mL, about 100 ng/mL, about 160 ng/mL, about 240 ng/mL, about 430 ng/mL, about 500 ng/mL, about 560 ng/mL, about 650 ng/mL, about 810 ng/mL, about 850 ng/mL, about 990 ng/mL, about 1460 ng/mL, or about 2000 ng/mL.

In some embodiments, the Cmax is achieved at about 0.5 hrs to about 5 hrs after administration (i.e. Tmax), about 0.5 hrs to about 4 hrs after administration, about 0.5 hrs to about 3 hrs after administration, about 0.5 hrs to about 2 hrs after administration, or about 0.5 hrs to about 1 hr after administration. Specific examples include about 0.5 hrs, about 1 hr, about 1.5 hrs, about 2 hrs, about 2.5 hrs, about 3 hrs, about 3.5 hrs, about 4 hrs, about 4.5 hrs, or about 5 hrs

In some embodiments, the compounds of Formula I, Formula II, and Formula III achieve a target area under the curve (herein after AUC) of about 10 ng·hr/mL to about 10,000 ng·hr/mL over a 24 hour period. In some embodiments, the compounds of Formula I, Formula II, and Formula III achieve an AUC of about 10 ng·hr/mL to about 8,000 ng·hr/mL over a 24 hour period. In some embodiments, the compounds of Formula I-III achieve an AUC of about 10 ng·hr/mL to about 6,000 ng·hr/mL over a 24 hour period. In some embodiments, the compounds of Formula I, Formula II, and Formula III achieve an AUC of about 10 ng·hr/mL to about 5,000 ng·hr/mL over a 24 hour period. In some embodiments, the compounds of Formula I, Formula II, and Formula III achieve an AUC of about 10 ng·hr/mL to about 4,000 ng·hr/mL over a 24 hour period. In some embodiments, the compounds of Formula I, Formula II, and Formula III achieve an AUC of about 10 ng·hr/mL to about 2,000 ng·hr/mL over a 24 hour period. In some embodiments, the compounds of Formula I-III achieve an AUC of about 10 ng·hr/mL to about 1,000 ng·hr/mL over a 24 hour period. In some embodiments, the compounds of Formula I, Formula II, and Formula III achieve an AUC of about 10 ng·hr/mL to about 500 ng·hr/mL over a 24 hour period.

In some embodiments, the compounds of Formula I, Formula II, and Formula III achieve an AUC of about 10 ng·hr/mL to about 1000 ng·hr/mL. In some embodiments, the compounds of Formula I, Formula II, and Formula III achieve an AUC of about 10 ng·hr/mL to about 800 ng·hr/mL. In some embodiments, the compounds of Formula I, Formula II, and Formula III achieve an AUC of about 10 ng·hr/mL to about 600 ng·hr/mL. In some embodiments, the compounds of Formula I, Formula II, and Formula III achieve an AUC of about 10 ng·hr/mL to about 500 ng·hr/mL. In some embodiments, the compounds of Formula I, Formula II, and Formula III achieve an AUC of about 10 ng·hr/mL to about 400 ng·hr/mL. In some embodiments, the compounds of Formula I, Formula II, and Formula III achieve an AUC of about 10 ng·hr/mL to about 200 ng·hr/mL. In some embodiments, the compounds of Formula I, Formula II, and Formula III achieve an AUC of about 10 ng·hr/mL to about 100 ng·hr/mL. In some embodiments, the compounds of Formula I, Formula II, and Formula III achieve an AUC of about 10 ng·hr/mL to about 50 ng·hr/mL.

The routes for administration (delivery) include, but are not limited to, one or more of oral (e.g., as a tablet, capsule, or as an ingestible solution), topical, mucosal (e.g., as a nasal spray or aerosol for inhalation), parenteral (e.g., by an injectable form), gastrointestinal, intraspinal, intraperitoneal, intramuscular, intravenous, intracerebroventricular, or other depot administration etc. Administration of an antibody or fragment will generally be by parenteral means.

Therefore, the compositions of the disclosure include those in a form especially formulated for, the mode of administration. In certain embodiments, the pharmaceutical compositions of the disclosure are formulated in a form that is suitable for oral delivery. For example compound CB and compound CF are sigma-2 receptor modulator compounds that are orally bioavailable in animal models and have been administered orally once per day and shown efficacy in a fear conditioning model. Orally bioavailable compounds as described herein can be prepared in an oral formulation. In some embodiments, the sigma-2 modulator compound is an orally bioavailable compound, suitable for oral delivery. In other embodiments, the pharmaceutical compositions of the disclosure are formulated in a form that is suitable for parenteral delivery. In some embodiments, the sigma-2 receptor modulator is an antibody or fragment thereof, wherein the antibody or fragment is formulated in a parenteral composition. For example, an anti-sigma-2 receptor antibody such as an anti-PGRMC1 antibody that blocks binding of Abeta oligomers to the sigma-2 receptor can be formulated for parenteral delivery.

The compounds of the disclosure may be formulated for administration in any convenient way for use in human or veterinary medicine and the disclosure therefore includes within its scope pharmaceutical compositions comprising a compound of the disclosure adapted for use in human or veterinary medicine. Such compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro edit. 1985). The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

There may be different composition/formulation requirements depending on the different delivery systems. It is to be understood that not all of the compounds need to be administered by the same route. Likewise, if the composition comprises more than one active component, then those components may be administered by different routes. By way of example, the pharmaceutical composition of the disclosure may be formulated to be delivered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be delivered by multiple routes.

The combination of a compound provided herein and an antibody or antibody fragment molecule can be formulated and administered by any of a number of routes and are administered at a concentration that is therapeutically effective in the indication or for the purpose sought. To accomplish this goal, the antibodies may be formulated using a variety of acceptable excipients known in the art. Typically, the antibodies are administered by injection, for example, intravenous injection. Methods to accomplish this administration are known to those of ordinary skill in the art. For example, Gokarn et al., 2008, J Pharm Sci 97(8):3051-3066, incorporated herein by reference, describe various high concentration antibody self buffered formulations. For example, monoclonal antibodies in self buffered formulation at e.g., 50 mg/mL mAb in 5.25% sorbitol, pH 5.0 or 60 mg/mL mAb in 5% sorbitol, 0.01% polysorbate 20, pH 5.2; or conventional buffered formulations, for example, 50 mg/mL mAb1 in 5.25% sorbitol, 25 or 50 mM acetate, glutamate or succinate, at pH 5.0; or 60 mg/mL in 10 mM acetate or glutamate, 5.25% sorbitol, 0.01% polysorbate 20, pH 5.2; other lower concentration formulations can be employed as known in the art.

Because compounds for use in the methods described herein cross the blood brain barrier they can be administered in a variety of methods including for example systemic (e.g., by iv, SC, oral, mucosal, transdermal route) or localized methods (e.g., intracranially). Where the compound of the disclosure is to be delivered mucosally through the gastrointestinal mucosa, it should be able to remain stable during transit though the gastrointestinal tract; for example, it should be resistant to proteolytic degradation, stable at acid pH and resistant to the detergent effects of bile. For example, the sigma-2 modulator compounds selected from the sigma-2 ligands and prepared for oral administration described above may be coated with an enteric coating layer. The enteric coating layer material may be dispersed or dissolved in either water or in a suitable organic solvent. As enteric coating layer polymers, one or more, separately or in combination, of the following can be used; e.g., solutions or dispersions of methacrylic acid copolymers, cellulose acetate phthalate, cellulose acetate butyrate, hydroxypropyl methylcellulose phthalate, hydroxypropyl methylcellulose acetate succinate, polyvinyl acetate phthalate, cellulose acetate trimellitate, carboxymethylethylcellulose, shellac or other suitable enteric coating layer polymer(s). For environmental reasons, an aqueous coating process may be preferred. In such aqueous processes methacrylic acid copolymers are most preferred.

Where appropriate, the pharmaceutical compositions can be administered by inhalation, by use of a skin patch, orally in the form of tablets containing excipients such as starch or lactose, or in capsules or ovules either alone or in admixture with excipients, or in the form of elixirs, solutions or suspensions containing flavoring or coloring agents, or they can be injected parenterally, for example intravenously, intramuscularly or subcutaneously. For buccal or sublingual administration the compositions may be administered in the form of tablets or lozenges, which can be formulated in a conventional manner.

Where the composition of the disclosure is to be administered parenterally, such administration includes without limitation: intravenously, intraarterially, intrathecally, intraventricularly, intracranially, intramuscularly or subcutaneously administering the compound of the disclosure; and/or by using infusion techniques. Antibodies or fragments are typically administered parenterally, for example, intravenously.

Pharmaceutical compositions suitable for injection or infusion may be in the form of a sterile aqueous solution, a dispersion or a sterile powder that contains the active ingredient, adjusted, if necessary, for preparation of such a sterile solution or dispersion suitable for infusion or injection. This preparation may optionally be encapsulated into liposomes. In all cases, the final preparation must be sterile, liquid, and stable under production and storage conditions. To improve storage stability, such preparations may also contain a preservative to prevent the growth of microorganisms. Prevention of the action of micro-organisms can be achieved by the addition of various antibacterial and antifungal agents, e.g., paraben, chlorobutanol, or ascorbic acid. In many cases isotonic substances are recommended, e.g., sugars, buffers and sodium chloride to assure osmotic pressure similar to those of body fluids, particularly blood. Prolonged absorption of such injectable mixtures can be achieved by introduction of absorption-delaying agents, such as aluminum monostearate or gelatin.

Dispersions can be prepared in a liquid carrier or intermediate, such as glycerin, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants.

For parenteral administration, the compound is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Sterile injectable solutions can be prepared by mixing a compound of Formula I, Formula II, or Formula III, with an appropriate solvent and one or more of the aforementioned carriers, followed by sterile filtering. In the case of sterile powders suitable for use in the preparation of sterile injectable solutions, preferable preparation methods include drying in vacuum and lyophilization, which provide powdery mixtures of the sigma-2 receptor modulators and desired excipients for subsequent preparation of sterile solutions.

The compounds according to the disclosure may be formulated for use in human or veterinary medicine by injection (e.g., by intravenous bolus injection or infusion or via intramuscular, subcutaneous or intrathecal routes) and may be presented in unit dose form, in ampoules, or other unit-dose containers, or in multi-dose containers, if necessary with an added preservative. The compositions for injection may be in the form of suspensions, solutions, or emulsions, in oily or aqueous vehicles, and may contain formulatory agents such as suspending, stabilizing, solubilizing and/or dispersing agents. Alternatively the active ingredient may be in sterile powder form for reconstitution with a suitable vehicle, e.g., sterile, pyrogen-free water, before use.

The compounds of the disclosure can be administered in the form of tablets, capsules, troches, ovules, elixirs, solutions or suspensions, for immediate-, delayed-, modified-, sustained-, pulsed- or controlled-release applications.

The compounds of the disclosure may also be presented for human or veterinary use in a form suitable for oral or buccal administration, for example in the form of solutions, gels, syrups, or suspensions, or a dry powder for reconstitution with water or other suitable vehicle before use. Solid compositions such as tablets, capsules, lozenges, troches, pastilles, pills, boluses, powder, pastes, granules, bullets or premix preparations may also be used. Solid and liquid compositions for oral use may be prepared according to methods well-known in the art.

Such compositions may also contain one or more pharmaceutically acceptable carriers and excipients which may be in solid or liquid form.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

The compositions may be administered orally, in the form of rapid or controlled release tablets, microparticles, mini tablets, capsules, sachets, and oral solutions or suspensions, or powders for the preparation thereof. Oral preparations may optionally include various standard pharmaceutical carriers and excipients, such as binders, fillers, buffers, lubricants, glidants, dyes, disintegrants, odorants, sweeteners, surfactants, mold release agents, antiadhesive agents and coatings. Some excipients may have multiple roles in the compositions, e.g., act as both binders and disintegrants.

Examples of pharmaceutically acceptable disintegrants for oral compositions useful in the disclosure include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and cross-linked polyvinylpyrrolidone.

Examples of pharmaceutically acceptable binders for oral compositions useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthine resin, alginates, magnesium-aluminum silicate, polyethylene glycol or bentonite.

Examples of pharmaceutically acceptable fillers for oral compositions include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulphate.

Examples of pharmaceutically acceptable lubricants useful in the compositions of the disclosure include, but are not limited to, magnesium stearate, talc, polyethylene glycol, polymers of ethylene oxide, sodium lauryl sulphate, magnesium lauryl sulphate, sodium oleate, sodium stearyl fumarate, and colloidal silicon dioxide.

Examples of suitable pharmaceutically acceptable odorants for the oral compositions include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Examples of useful pharmaceutically acceptable coatings for the oral compositions, typically used to facilitate swallowing, modify the release properties, improve the appearance, and/or mask the taste of the compositions include, but are not limited to, hydroxypropylmethylcellulose, hydroxypropylcellulose and acrylate-methacrylate copolymers.

Suitable examples of pharmaceutically acceptable sweeteners for the oral compositions include, but are not limited to, aspartame, saccharin, saccharin sodium, sodium cyclamate, xylitol, mannitol, sorbitol, lactose and sucrose.

Suitable examples of pharmaceutically acceptable buffers include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants include, but are not limited to, sodium lauryl sulphate and polysorbates.

Solid compositions of a similar type may also be employed as fillers in gelatin capsules.

Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

As indicated, the compounds of the disclosure can be administered intranasally or by inhalation and is conveniently delivered in the form of a dry powder inhaler or an aerosol spray presentation from a pressurized container, pump, spray or nebulizer with the use of a suitable propellant, e.g., dichlorodifluoromethane, trichlorofluoromethane, dichlorotetrafluoroethane, a hydrofluoroalkane such as 1,1,1,2-tetrafluoroethane (HFA 134AT) or 1,1,1,2,3,3,3-heptafluoropropane (HFA 227EA), carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e.g., using a mixture of ethanol and the propellant as the solvent, which may additionally contain a lubricant, e.g., sorbitan trioleate.

Capsules and cartridges (made, for example, from gelatin) for use in an inhaler or insufflator may be formulated to contain a powder mix of the compound and a suitable powder base such as lactose or starch.

For topical administration by inhalation the compounds according to the disclosure may be delivered for use in human or veterinary medicine via a nebulizer.

The pharmaceutical compositions of the disclosure may contain from 0.01 to 99% weight per volume of the active material. For topical administration, for example, the composition will generally contain from 0.01-10%, more preferably 0.01-1% of the active material.

The compounds can also be administered in the form of liposome delivery systems, such as small unilamellar vesicles, large unilamellar vesicles and multilamellar vesicles.

Liposomes can be formed from a variety of phospholipids, such as cholesterol, stearylamine or phosphatidylcholines.

The pharmaceutical composition or unit dosage form of the disclosure may be administered according to a dosage and administration regimen defined by routine testing in the light of the guidelines given above in order to obtain optimal activity while minimizing toxicity or side effects for a particular patient. However, such fine tuning of the therapeutic regimen is routine in the light of the guidelines given herein.

The dosage of the compounds of the disclosure may vary according to a variety of factors such as underlying disease conditions, the individual's condition, weight, sex and age, and the mode of administration. An effective amount for treating a disorder can easily be determined by empirical methods known to those of ordinary skill in the art, for example by establishing a matrix of dosages and frequencies of administration and comparing a group of experimental units or subjects at each point in the matrix. The exact amount to be administered to a patient will vary depending on the state and severity of the disorder and the physical condition of the patient. A measurable amelioration of any symptom or parameter can be determined by a person skilled in the art or reported by the patient to the physician. It will be understood that any clinically or statistically significant attenuation or amelioration of any symptom or parameter of urinary tract disorders is within the scope of the disclosure. Clinically significant attenuation or amelioration means perceptible to the patient and/or to the physician.

The amount of the compound to be administered can range between about 0.01 and about 25 mg/kg/day, usually between about 0.1 and about 10 mg/kg/day and most often between 0.2 and about 5 mg/kg/day. It will be understood that the pharmaceutical formulations of the disclosure need not necessarily contain the entire amount of the compound that is effective in treating the disorder, as such effective amounts can be reached by administration of a plurality of divided doses of such pharmaceutical formulations.

In a preferred embodiment of the disclosure, the compounds of Formula I, Formula II, and Formula III are formulated in capsules or tablets, usually containing 10 to 200 mg of the compounds of the disclosure, and are preferably administered to a patient at a total daily dose of 10 to 300 mg, preferably 20 to 150 mg and most preferably about 50 mg, about 100 mg, and about 300 mg.

A pharmaceutical composition for parenteral administration contains from about 0.01% to about 100% by weight of the active compound of the disclosure, based upon 100% weight of total pharmaceutical composition.

Generally, transdermal dosage forms contain from about 0.01% to about 100% by weight of the active compound versus 100% total weight of the dosage form.

The pharmaceutical composition or unit dosage form may be administered in a single daily dose, or the total daily dosage may be administered in divided doses. In addition, co-administration or sequential administration of another compound for the treatment of the disorder may be desirable. To this purpose, the combined active principles are formulated into a simple dosage unit.

### Synthesis of the Compounds

Compounds of Formula I, Formula II, and Formula III and enantiomers, diastereomers, N-oxides, and pharmaceutically acceptable salts thereof, may be prepared by the general methods outlined in, for example, WO2013/029057, incorporated herein by reference, or as described hereinafter, said methods constituting a further aspect of the disclosure.

It will be appreciated by those skilled in the art that it may be desirable to use protected derivatives of intermediates used in the preparation of the compounds. Protection and deprotection of functional groups may be performed by methods known in the art (see, for example, Green and Wuts Protective Groups in Organic Synthesis. John Wiley and Sons, New York, 1999.). Hydroxy or amino groups may be protected with any hydroxy or amino protecting group. The amino protecting groups may be removed by conventional techniques. For example, acyl groups, such as alkanoyl, alkoxycarbonyl and aroyl groups, may be removed by solvolysis, e.g., by hydrolysis under acidic or basic conditions. Arylmethoxycarbonyl groups (e.g., benzyloxycarbonyl) may be cleaved by hydrogenolysis in the presence of a catalyst such as palladium-on-charcoal.

The synthesis of the target compounds is completed by removing any protecting groups which may be present in the penultimate intermediates using standard techniques, which are well-known to those skilled in the art. The deprotected final products are then purified, as necessary, using standard techniques such as silica gel chromatography, HPLC on silica gel and the like, or by recrystallization.

### Methods

Changes in Biomarker Expression after Treating a Subject with Alzheimer's Disease

Embodiments of the present disclosure are directed to a method of treating Alzheimer's disease in a subject in need thereof, comprising administering to the subject a therapeutically effective amount of a compound, or a pharmaceutically acceptable salt thereof, according to Formula I:

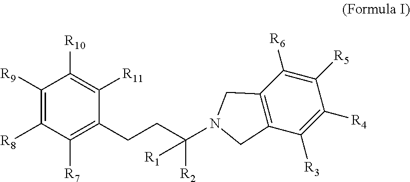

wherein: R₁ and R₂ are each independently selected from H, C₁-C₆ alkyl, or CH₂OR′; where R′═H or C₁-C₆ alkyl;
R₃, R₄, R₅, and R₆ are each independently selected from H, C₁-C₆ alkyl, OH, OCH₃, OCH(CH₃)₂, OCH₂CH(CH₃)₂, OC(CH₃)₃, O(C₁-C₆ alkyl), OCF₃, OCH₂CH₂OH, O(C₁-C₆ alkyl)OH, O(C₁-C₆ haloalkyl), F, Cl, Br, I, CF₃, CN, NO₂, NH₂, C₁-C₆ haloalkyl, C₁-C₆ hydroxyalkyl, C₁₋₆ alkoxy C₁₋₆alkyl, aryl, heteroaryl, C₃₋₇ cycloalkyl, heterocycloalkyl, alkylaryl, heteroaryl, CO₂R′, C(O)R′, NH(C₁₋₄ alkyl), N(C₁₋₄ alkyl)₂, NH(C₃₋₇ cycloalkyl), NHC(O)(C₁₋₄ alkyl), CONR′₂, NC(O)R′, NS(O)ₙR′, S(O)ₙNR′₂, S(O)ₙR′, C(O)O(C₁₋₄ alkyl), OC(O)N(R′)₂, C(O) (C₁₋₄ alkyl), and C(O)NH(C₁₋₄ alkyl); where n=0, 1, or 2; R′ are each independently H, CH₃, CH₂CH₃, C₃-C₆ alkyl, C₁-C₆ haloalkyl; or optionally substituted aryl, alkylaryl, piperazin-1-yl, piperidin-1-yl, morpholinyl, heterocycloalkyl, heteroaryl, C₁₋₆ alkoxy, NH(C₁₋₄ alkyl), or NH(C₁₋₄ alkyl)₂, wherein optionally substituted group is selected from C₁-C₆ alkyl or C₂-C₇ acyl;
or R₃ and R₄, together with the C atom to which they are attached form a form a 4-5-, 6- 7- or 8-membered cycloalkyl, aryl, heteroaryl, or heterocycloalkyl that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R³ and R⁴, or R⁴ and R⁵, are each independently selected from a bond, C, N, S, and O; or R₃ and R₄ are linked together to form a —O—C₁₋₂ methylene-O— group;
or R₄ and R₅, together with the C atom to which they are attached form a form a 4-, 5-, 6- 7- or 8-membered cycloalkyl, aryl, heteroaryl, or heterocycloalkyl that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R³ and R⁴, or R⁴ and R⁵, are each independently selected from a bond, C, N, S, and O; or R₄ and R₅ are linked together to form a —O—C₁₋₂ methylene-O— group;
R₇, R₈, R₉, R₁₀, and R₁₁ are each independently selected from H, C₁-C₆ alkyl, OH, OCH₃, OCH(CH₃)₂, OCH₂CH(CH₃)₂, OC(CH₃)₃, O(C₁-C₆ alkyl), OCF₃, OCH₂CH₂OH, O(C₁-C₆ alkyl)OH, O(C₁-C₆ haloalkyl), O(CO)R′, F, Cl, Br, I, CF₃, CN, NO₂, NH₂, C₁-C₆ haloalkyl, C₁-C₆ hydroxyalkyl, C₁₋₆ alkoxy C₁₋₆alkyl, aryl, heteroaryl, C₃₋₇ cycloalkyl, heterocycloalkyl, alkylaryl, heteroaryl, CO₂R′, C(O)R′, NH(C₁₋₄ alkyl), N(C₁₋₄ alkyl)₂, NH(C₃₋₇ cycloalkyl), NHC(O)(C₁₋₄ alkyl), CONR′₂, NC(O)R′, NS(O)ₙR′, S(O)ₙNR′₂, S(O)ₙR′, C(O)O(C₁₋₄ alkyl), OC(O)N(R′)₂, C(O) (C₁₋₄ alkyl), and C(O)NH(C₁₋₄ alkyl); where n=0, 1, or 2; R′ are each independently H, CH₃, CH₂CH₃, C₃-C₆ alkyl, C₁-C₆ haloalkyl, aryl, alkylaryl, piperazin-1-yl, piperidin-1-yl, morpholinyl, heterocycloalkyl, heteroaryl, C₁₋₆ alkoxy, NH(C₁₋₄ alkyl), or NH(C₁₋₄ alkyl)₂;
or R₇ and R₈, together with the N or C atoms to which they are attached form a form a 4-, 5-, 6- 7- or 8-membered cycloalkyl, aryl, heterocycloalkyl or heteroaryl group that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R⁹ and R¹⁰ are each independently selected from a bond, C, N, S, and O; or R₇ and R₈ are linked together to form a —O—C₁₋₂ methylene-O— group;
or R₈ and R₉, together with the N or C atoms to which they are attached form a form a 4-, 5-, 6- 7- or 8-membered cycloalkyl, aryl, heterocycloalkyl or heteroaryl group that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R⁹ and R¹⁰ are each independently selected from a bond, C, N, S, and O; or R₈ and R₉ are linked together to form a —O—C₁₋₂ methylene-O— group,
wherein each of the O, C₁₋₆ alkyl, C₁₋₆ haloalkyl, heteroaryl, aryl, heteroaryl, heterocycloalkyl, and cycloalkyl is optionally independently substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl and heterocycloalkyl;
with the proviso that the following compounds are excluded:

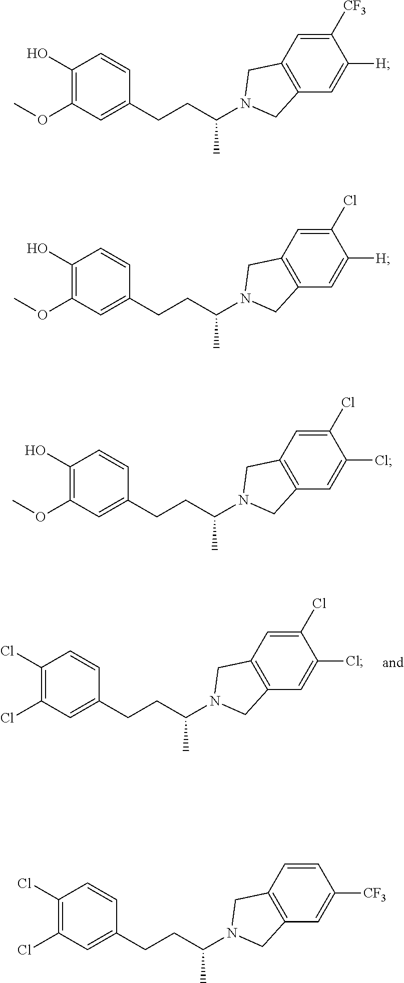

In some embodiments of the method, the compound of Formula I is:

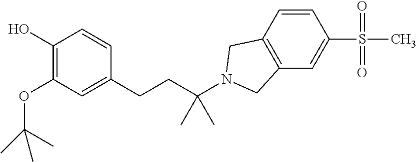 [SMILES: CC(C)(C)Oc1cc(CCC(C)(C)N2Cc3ccc(S(C)(=O)=O)cc3C2)ccc1O]

or a pharmaceutically acceptable sale thereof. In some embodiments, the salt is fumarate salt.

Embodiments of the present disclosure are directed to a method of treating Alzheimer's disease in a subject in need thereof, comprising administering to the subject a therapeutically effective amount of a compound, or a pharmaceutically acceptable salt thereof, according to a compound or pharmaceutical composition according to any embodiment described herein.

In some embodiments, the administration of a compound or pharmaceutical composition according to any embodiment described herein results in a decrease in the expression of at least one biomarker, an increase in the expression of at least one biomarker of the subject in need thereof, or a combination of both. In some embodiments, the administration of a compound of Formula I results in a decrease in the expression of at least one biomarker, an increase in the expression of at least one biomarker of the subject in need thereof, or a combination of both. In some embodiments, the administration of a compound of Formula I, Formula II, or Formula III results in a decrease in the expression of at least one biomarker, an increase in the expression of at least one biomarker of the subject in need thereof, or a combination of both.

In some embodiments, the at least one biomarker with decreased expression is selected from the group consisting of chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL) and any combination thereof.

In some embodiments, the at least one biomarker with increased expression is selected from the group consisting of GDI1, SMOC1, YWHAZ, YWHAG, MAPT, CHI3L1, FABP3, GAP43, NRGN, NEFL, TREM2, and any combination thereof.

In some embodiments, the subject has been diagnosed with Alzheimer's disease. In some embodiments, the subject does not exhibit any detectable clinical symptoms of Alzheimer's disease. In some embodiments, the subject has no cognitive impairment. In some embodiments, the subject has been diagnosed with mild cognitive impairment. In some embodiments, the subject has been diagnosed with mild Alzheimer's disease. In some embodiments, the subject has been diagnosed with mild to moderate Alzheimer's disease. In some embodiments, the subject has been diagnosed with moderate Alzheimer's disease. In some embodiments, the subject has been diagnosed with moderate to severe Alzheimer's disease. In some embodiments, the subject has been diagnosed with severe Alzheimer's disease.

In some embodiments, the subject is a mammal. In some embodiments, the subject is a non-human mammal. In some embodiments, the subject is a human. In some embodiments, the subject is a human with a diagnosis of Alzheimer's disease.

Alzheimer's disease (AD) is defined histologically by the presence of extracellular β-amyloid (Aβ) plaques and intraneuronal neurofibrillary tangles in the cerebral cortex. Various diagnostic and prognostic biomarkers are known in the art, such as magnetic resonance imaging, single photon emission tomography, FDG PET, PiB PET, CSF tau and Abeta analysis, as well as available data on their diagnostic accuracy are discussed in Alves et al., 2012, Alzheimer's disease: a clinical practice-oriented review, Frontiers in Neurology, April, 2012, vol 3, Article 63, 1-20, which is incorporated herein by reference.

The diagnosis of dementia, along with the prediction of who will develop dementia, has been assisted by magnetic resonance imaging and positron emission tomography (PET) by using [(18)F]fluorodeoxyglucose (FDG). These techniques are not specific for AD. See, e.g., Vallabhajosula S. Positron emission tomography radiopharmaceuticals for imaging brain Beta-amyloid. Semin Nucl Med. 2011 July; 41(4):283-99. Another PET ligand recently FDA approved for imaging moderate to frequent amyloid neuritic plaques in patients with cognitive impairment is Florbetapir F 18 injection, (4-((1E)-2-(6-{2-(2-(2-(18F)fluoroethoxy)ethoxy)ethoxy}pyridin-3-yl)ethenyl)-N-methylbenzenamine, AMYVID®, Lilly). Florbetapir binds specifically to fibrillar Abeta, but not to neurofibrillary tangles. See, e.g., Choi S R, et al., Correlation of amyloid PET ligand florbetapir F 18 binding with Aβ aggregation and neuritic plaque deposition in postmortem brain tissue. Alzheimer Dis Assoc Disord. 2012 January; 26(1):8-16. The PET ligand florbetapir suffers from low specificity with respect to qualitative visual assessment of the PET scans. Camus et al., 2012, Eur J Nucl Med Mol Imaging 39:621-631. However, many people with neuritic plaques seem cognitively normal.

CSF markers for Alzheimer's disease include total tau, phosphor-tau and Abeta42. See, for example, Andreasen, Sjogren and Blennow, World J Biol Psyciatry, 2003, 4(4): 147-155, which is incorporated herein by reference. Reduced CSF levels of the 42 amino acid fon of Abeta (Abeta42) and increased CSF levels of total tau in Alzheimer's disease have been found in numerous studies. In addition, there are known genetic markers for mutations in the APP gene useful in the identification of subjects at risk for developing AD. See, for example, Goate et al., “Segregation of a missense mutation in the amyloid precursor protein gene with familial Alzheimer's disease,” Nature, 349, 704-706, 1991, which is incorporated herein by reference. In embodiments, any known diagnostic or prognostic method can be employed to identify a subject having or at risk of having Alzheimer's disease.

In some embodiments, the subject is aged between 50 and 80 years. In some embodiments the subject is younger than 50 years. In some embodiments, the subject is aged 80 years old or older.

In some embodiments, the subject has an MMSE score between about 18-26. In some embodiments, the subject has an MMSE score between about 18-26, corresponding to mild to moderate cognitive impairment. In some embodiments, the subject has an MMSE score greater than or equal to 24. In some embodiments, the subject has an MMSE score greater than or equal to 24, corresponding to mild cognitive impairment to normal cognitive ability. In some embodiments, the subject has an MMSE score between about 10 to about 18. In some embodiments, the subject has an MMSE score between about 10 to about 18, corresponding moderate to severe Alzheimer's disease. In some embodiments, the subject has mild Alzheimer's disease. In some embodiments, the subject has mild to moderate Alzheimer's disease. In some embodiments, the subject has moderate to severe Alzheimer's disease. In some embodiments, the subject does not exhibit any detectable clinical symptoms of Alzheimer's disease. In some embodiments, the subject has no cognitive impairment.

In some embodiments, a compound or pharmaceutical composition according to any embodiment described herein is administered for at least about 6 months. In some embodiments, a compound of Formula I, Formula II, and/or Formula III is administered for at least about 6 months.

In some embodiments, the therapeutically effective amount of a compound or pharmaceutical composition according to any embodiment described herein is from about 0.0001 mg to about 2000 mg, about 0.0001 mg to about 1500 mg, about 0.0001 mg to about 1200 mg, about 0.0001 mg to about 1000 mg, about 0.0001 mg to about 800 mg, about 0.0001 mg to about 500 mg, about 0.0001 mg to about 250 mg, about 0.0001 mg to about 200 mg, or about 0.0001 mg to about 100 mg. In some embodiments, the therapeutically effective amount of a compound or pharmaceutical composition according to any embodiment described herein is about 100 mg to about 300 mg. In some embodiments, the therapeutically effective amount is about 1 mg, about 5 mg, about 10 mg, about 25 mg, about 30 mg, about 50 mg, about 90 mg, about 100 mg, about 180 mg, about 280 mg, about 300 mg, about 450 mg, about 560 mg, about 840 mg, about 1120 mg, about 1500 mg, about 2000 mg.

In some embodiments, a compound or pharmaceutical composition of according to any of the embodiments described herein is administered for at least about 6 months. In some embodiments, a compound or pharmaceutical composition of according to any of the embodiments described herein is administered daily for about 6 months. In some embodiments, the compound of Formula I, Formula II, and/or Formula III is administered for at least about 6 months. In some embodiments, the therapeutically effective amount of the compound of Formula I, Formula II, and/or Formula III is administered daily for about 6 months.

Some embodiments are directed to methods of screening for compounds that may be useful in the treatment and/or prevention of Alzheimer's disease comprising: (a) measuring the level of at least one biomarker selected from the group consisting of chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL) and any combination thereof in a first biological sample obtained from a test subject; (b) administering the test compound to the test subject; (c) measuring the level of the at least one biomarker after administration of the test compound in a second biological sample from the test subject; and (d) correlating a decrease or increase in the expression of at least one biomarker selected from the group consisting of chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL) and any combination thereof, with potential therapeutic efficacy of the test compound. In some embodiments, a test compound with potential therapeutic efficacy will result in a decrease in the expression of at least one biomarker selected from the group consisting of chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL) and any combination thereof.

Some embodiments are directed to methods of screening for compounds that may be useful in the treatment and/or prevention of Alzheimer's disease comprising: (a) measuring the level of at least one biomarker selected from the group consisting of chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL) and any combination thereof in a first biological sample obtained from a test subject; (b) administering the test compound to the test subject; (c) measuring the level of the at least one biomarker after administration of the test compound in a second biological sample from the test subject; and (d) correlating a decrease or increase in the expression of at least one biomarker selected from the group consisting of chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL) and any combination thereof with potential therapeutic efficacy of the test compound. In some embodiments, a test compound with potential therapeutic efficacy will result in an decrease in the expression of at least one biomarker selected from the group consisting of chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and any combination thereof.

In some embodiments, the at least one biomarker is a protein. In some embodiments, the at least one biomarker is a network of proteins. In some embodiments, the at least one biomarker is a ribonucleic acid (RNA). In some embodiments, the at least one biomarker is a network of RNA. In some embodiments, the at least one biomarker is measured longitudinally across patients.

In some embodiments, the at least one biomarker is measured by Liquid chromatography-mass spectrometry. In some embodiments, the at least one biomarker is measured by mass spectrometry. In some embodiments, the mass spectrometry is SELDI-MS. In some embodiments, the level of at the at least one biomarker is measured by immunoassay. In some embodiments, the level of at the at least one biomarker is measured by a weighted gene co-expression network analysis (WGCNA).

In some embodiments, the sample is blood or a blood derivative. In some embodiments, the blood derivative is serum. In some embodiments, the sample is cerebrospinal fluid. In some embodiments, the correlating is performed by executing a software classification algorithm. In some embodiments, the subject is a cell capable of expressing chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and any combination thereof at levels that mimic Alzheimer's disease. In some embodiments, the subject is a cell capable of expressing chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), or a combination thereof at levels that mimic Alzheimer's disease.

Treating a Subject with Alzheimer's Disease

Embodiments of the present disclosure are directed to a method of treating Alzheimer's disease in a subject in need thereof, comprising administering to the subject a therapeutically effective amount of a compound, or a pharmaceutically acceptable salt thereof, according to Formula I:

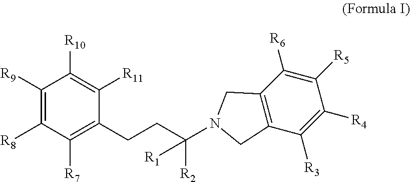

wherein: R₁ and R₂ are each independently selected from H, C₁-C₆ alkyl, or CH₂OR′; where R′═H or C₁-C₆ alkyl;
R₃, R₄, R₅, and R₆ are each independently selected from H, C₁-C₆ alkyl, OH, OCH₃, OCH(CH₃)₂, OCH₂CH(CH₃)₂, OC(CH₃)₃, O(C₁-C₆ alkyl), OCF₃, OCH₂CH₂OH, O(C₁-C₆ alkyl)OH, O(C₁-C₆ haloalkyl), F, Cl, Br, I, CF₃, CN, NO₂, NH₂, C₁-C₆ haloalkyl, C₁-C₆ hydroxyalkyl, C₁₋₆ alkoxy C₁₋₆alkyl, aryl, heteroaryl, C₃₋₇ cycloalkyl, heterocycloalkyl, alkylaryl, heteroaryl, CO₂R′, C(O)R′, NH(C₁₋₄ alkyl), N(C₁₋₄ alkyl)₂, NH(C₃₋₇ cycloalkyl), NHC(O)(C₁₋₄ alkyl), CONR′₂, NC(O)R′, NS(O)ₙR′, S(O)ₙNR′₂, S(O)ₙR′, C(O)O(C₁₋₄ alkyl), OC(O)N(R′)₂, C(O) (C₁₋₄ alkyl), and C(O)NH(C₁₋₄ alkyl); where n=0, 1, or 2; R′ are each independently H, CH₃, CH₂CH₃, C₃-C₆ alkyl, C₁-C₆ haloalkyl; or optionally substituted aryl, alkylaryl, piperazin-1-yl, piperidin-1-yl, morpholinyl, heterocycloalkyl, heteroaryl, C₁₋₆ alkoxy, NH(C₁₋₄ alkyl), or NH(C₁₋₄ alkyl)₂, wherein optionally substituted group is selected from C₁-C₆ alkyl or C₂-C₇ acyl;
or R₃ and R₄, together with the C atom to which they are attached form a form a 4-, 5-, 6- 7- or 8-membered cycloalkyl, aryl, heteroaryl, or heterocycloalkyl that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R³ and R⁴, or R⁴ and R⁵, are each independently selected from a bond, C, N, S, and O; or R₃ and R₄ are linked together to form a —O—C₁₋₂ methylene-O— group;
or R₄ and R₅, together with the C atom to which they are attached form a form a 4-, 5-, 6- 7- or 8-membered cycloalkyl, aryl, heteroaryl, or heterocycloalkyl that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R³ and R⁴, or R⁴ and R⁵, are each independently selected from a bond, C, N, S, and O; or R₄ and R₅ are linked together to form a —O—C₁₋₂ methylene-O— group;
R₇, R₈, R₉, R₁₀, and R₁₁ are each independently selected from H, C₁-C₆ alkyl, OH.

OCH₃, OCH(CH₃)₂, OCH₂CH(CH₃)₂, OC(CH₃)₃, O(C₁-C₆ alkyl), OCF₃, OCH₂CH₂OH, O(C₁-C₆ alkyl)OH, O(C₁-C₆ haloalkyl), O(CO)R′, F, Cl, Br, I, CF₃, CN, NO₂, NH₂, C₁-C₆ haloalkyl, C₁-C₆ hydroxyalkyl, C₁₋₆ alkoxy C₁₋₆alkyl, aryl, heteroaryl, C₃₋₇ cycloalkyl, heterocycloalkyl, alkylaryl, heteroaryl, CO₂R′, C(O)R′, NH(C₁₋₄ alkyl), N(C₁₋₄ alkyl)₂, NH(C₃₋₇ cycloalkyl), NHC(O)(C₁₋₄ alkyl), CONR′₂, NC(O)R′, NS(O)ₙR′, S(O)ₙNR′₂, S(O)ₙR′, C(O)O(C₁₋₄ alkyl), OC(O)N(R′)₂, C(O) (C₁₋₄ alkyl), and C(O)NH(C₁₋₄ alkyl); where n=0, 1, or 2; R′ are each independently H, CH₃, CH₂CH₃, C₃-C₆ alkyl, C₁-C₆ haloalkyl, aryl, alkylaryl, piperazin-1-yl, piperidin-1-yl, morpholinyl, heterocycloalkyl, heteroaryl, C₁₋₆ alkoxy, NH(C₁₋₄ alkyl), or NH(C₁₋₄ alkyl)₂;
or R₇ and R₈, together with the N or C atoms to which they are attached form a form a 4-, 5-, 6- 7- or 8-membered cycloalkyl, aryl, heterocycloalkyl or heteroaryl group that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R⁹ and R¹⁰ are each independently selected from a bond, C, N, S, and O; or R₇ and R₈ are linked together to form a —O—C₁₋₂ methylene-O— group;
or R₈ and R₉, together with the N or C atoms to which they are attached form a form a 4-, 5-, 6- 7- or 8-membered cycloalkyl, aryl, heterocycloalkyl or heteroaryl group that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R⁹ and R¹⁰ are each independently selected from a bond, C, N, S, and O; or R₈ and R₉ are linked together to form a —O—C₁₋₂ methylene-O— group,
wherein each of the O, C₁₋₆ alkyl, C₁₋₆ haloalkyl, heteroaryl, aryl, heteroaryl, heterocycloalkyl, and cycloalkyl is optionally independently substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl and heterocycloalkyl;
with the proviso that the following compounds are excluded:

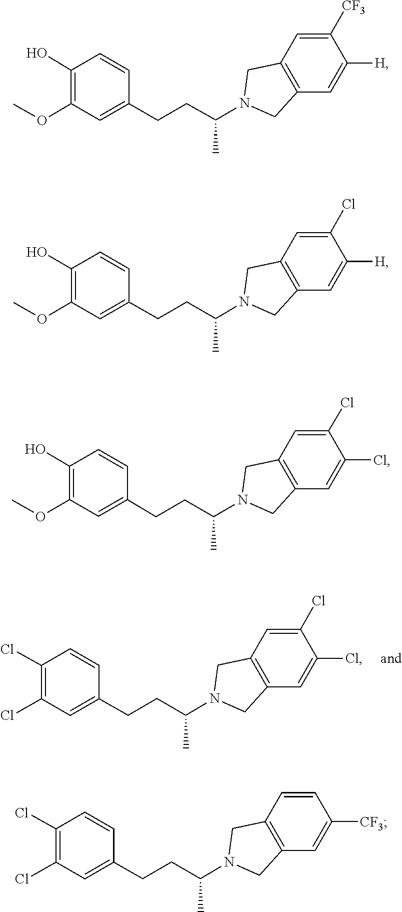

In some embodiments of the method, the compound of Formula I is:

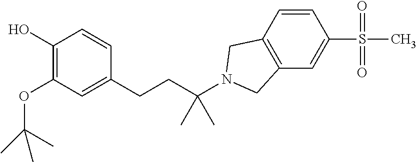 [SMILES: CC(C)(C)Oc1cc(CCC(C)(C)N2Cc3ccc(S(C)(=O)=O)cc3C2)ccc1O]

or a pharmaceutically acceptable sale thereof. In some embodiments, the salt is fumarate salt.

Embodiments of the present disclosure are directed to a method of treating Alzheimer's disease in a subject in need thereof, comprising administering to the subject a therapeutically effective amount of a compound, or a pharmaceutically acceptable salt thereof, according to a compound or pharmaceutical composition according to any embodiment described herein.

In some embodiments, the subject in need thereof, prior to administration of a compound or pharmaceutical composition according to any embodiment described herein, has a decrease in the expression level of at least one biomarker, an increase in the expression of at least one biomarker. In some embodiments, the subject in need thereof, prior to administration of a compound of Formula I, has a decrease in the expression of at least one biomarker of the subject in need thereof, an increase in the expression of at least one biomarker of the subject in need thereof, or a combination of both. In some embodiments, the subject in need thereof, prior to administration of a compound of Formula I, Formula II, and/or Formula III, has a decrease in the expression of at least one biomarker of the subject in need thereof, an increase in the expression of at least one biomarker of the subject in need thereof, or a combination of both.

In some embodiments, the at least one biomarker with increased expression prior to administration of a compound or pharmaceutical composition according to any embodiment described herein is selected from the group consisting of chitinase 3 like 1 (CHI3L1). SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and any combination thereof.

In some embodiments, the at least one biomarker with increased expression prior to administration of a compound or pharmaceutical composition according to any embodiment described herein is selected from the group consisting of chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and any combination thereof.

In some embodiments, the subject has been diagnosed with Alzheimer's disease. In some embodiments, the subject does not exhibit any detectable clinical symptoms of Alzheimer's disease. In some embodiments, the subject has no cognitive impairment. In some embodiments, the subject has been diagnosed with mild cognitive impairment. In some embodiments, the subject has been diagnosed with mild Alzheimer's disease. In some embodiments, the subject has been diagnosed with mild to moderate Alzheimer's disease. In some embodiments, the subject has been diagnosed with moderate Alzheimer's disease. In some embodiments, the subject has been diagnosed with moderate to severe Alzheimer's disease. In some embodiments, the subject has been diagnosed with severe Alzheimer's disease.

In some embodiments, the administration of a compound or pharmaceutical composition according to any embodiments disclosed herein to a subject with no detectable clinical symptoms of Alzheimer's disease returns at least one biomer to baseline levels. In some embodiments, the administration of a compound or pharmaceutical composition according to any embodiments disclosed herein to a subject with no detectable clinical symptoms of Alzheimer's disease significantly improves biomarker levels toward baseline levels.

In some embodiments, the subject is a mammal. In some embodiments, the subject is a non-human mammal. In some embodiments, the subject is a human. In some embodiments, the subject is a human with a diagnosis of Alzheimer's disease.

Alzheimer's disease (AD) is defined histologically by the presence of extracellular β-amyloid (Aβ) plaques and intraneuronal neurofibrillary tangles in the cerebral cortex. Various diagnostic and prognostic biomarkers are known in the art, such as magnetic resonance imaging, single photon emission tomography, FDG PET, PiB PET, CSF tau and Abeta analysis, as well as available data on their diagnostic accuracy are discussed in Alves et al., 2012, Alzheimer's disease: a clinical practice-oriented review, Frontiers in Neurology, April, 2012, vol 3, Article 63, 1-20, which is incorporated herein by reference.

The diagnosis of dementia, along with the prediction of who will develop dementia, has been assisted by magnetic resonance imaging and positron emission tomography (PET) by using [(18)F]fluorodeoxyglucose (FDG). These techniques are not specific for AD. See, e.g., Vallabhajosula S. Positron emission tomography radiopharmaceuticals for imaging brain Beta-amyloid. Semin Nucl Med. 2011 July; 41(4):283-99. Another PET ligand recently FDA approved for imaging moderate to frequent amyloid neuritic plaques in patients with cognitive impairment is Florbetapir F 18 injection, (4-((1E)-2-(6-{2-(2-(2-(18F)fluoroethoxy)ethoxy)ethoxy}pyridin-3-yl)ethenyl)-N-methylbenzenamine, AMYVID®, Lilly). Florbetapir binds specifically to fibrillar Abeta, but not to neurofibrillary tangles. See, e.g., Choi S R, et al., Correlation of amyloid PET ligand florbetapir F 18 binding with Aβ aggregation and neuritic plaque deposition in postmortem brain tissue. Alzheimer Dis Assoc Disord. 2012 January; 26(1):8-16. The PET ligand florbetapir suffers from low specificity with respect to qualitative visual assessment of the PET scans. Camus et al., 2012, Eur J Nucl Med Mol Imaging 39:621-631. However, many people with neuritic plaques seem cognitively normal.

CSF markers for Alzheimer's disease include total tau, phosphor-tau and Abeta42. See, for example, Andreasen, Sjogren and Blennow, World J Biol Psyciatry, 2003, 4(4): 147-155, which is incorporated herein by reference. Reduced CSF levels of the 42 amino acid fon of Abeta (Abeta42) and increased CSF levels of total tau in Alzheimer's disease have been found in numerous studies. In addition, there are known genetic markers for mutations in the APP gene useful in the identification of subjects at risk for developing AD. See, for example, Goate et al., Segregation of a missense mutation in the amyloid precursor protein gene with familial Alzheimer's disease, Nature, 349, 704-706, 1991, which is incorporated herein by reference. In embodiments, any known diagnostic or prognostic method can be employed to identify a subject having or at risk of having Alzheimer's disease.

Disclosed herein are biomarker combinations that are useful in qualifying Alzheimer's disease status in a patient as well as identifying therapeutic interventions that may be useful in treating and/or preventing the progression of Alzheimer's disease in a patient. In some embodiments, the biomarker is a protein-based biomarker. In some embodiments, the biomarker may be selected from the group consisting of chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and any combination thereof.

Some embodiments are directed to methods for qualifying Alzheimer's disease status in a subject comprising measuring at least one biomarker in a biological sample from the subject, wherein at least one biomarker is selected from the group consisting of chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and any combination thereof, and correlating the measurement or measurements with an Alzheimer's disease status selected from Alzheimer's disease and non-Alzheimer's disease. In some embodiments, a plurality of biomarkers in the biological sample are measured, wherein the measured biomarkers comprise at least two biomarkers selected from the group consisting of chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and combination thereof. In some embodiments, a change in protein expression that differs from that of a healthy patient is indicative of a status of Alzheimer's disease. In some embodiments, a status of Alzheimer's disease may be demonstrated by an increase in the expression of at least one biomarker selected from the group consisting of chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), or any combination thereof.

In some embodiments, a status of Alzheimer's disease may be demonstrated by a decrease in the expression of at least one biomarker, an increase in the expression of at least one biomarker, or a combination thereof. In some embodiments, the decrease or increase in the expression of a biomarker is determined by comparing expression of a biomarker in the subject with expression of the same biomarker in a healthy patient. In some embodiments, the decrease or increase in the expression of a biomarker is determined by measuring the expression of the same biomarker in the same patient but at an earlier point in time.

In some embodiments, the subject is aged between 50 and 80 years. In some embodiments the subject is younger than 50 years. In some embodiments, the subject is aged 80 years old or older.

In some embodiments, the subject has an MMSE score between about 18-26. In some embodiments, the subject has an MMSE score between about 18-26, corresponding to mild to moderate cognitive impairment. In some embodiments, the subject has an MMSE score greater than or equal to 24. In some embodiments, the subject has an MMSE score greater than or equal to 24, corresponding to mild cognitive impairment to normal cognitive ability. In some embodiments, the subject has an MMSE score between about 10 to about 18. In some embodiments, the subject has an MMSE score between about 10 to about 18, corresponding moderate to severe Alzheimer's disease. In some embodiments, the subject has mild Alzheimer's disease. In some embodiments, the subject has mild to moderate Alzheimer's disease. In some embodiments, the subject has moderate to severe Alzheimer's disease. In some embodiments, the subject does not exhibit any detectable clinical symptoms of Alzheimer's disease. In some embodiments, the subject has no cognitive impairment.

In some embodiments, a compound or pharmaceutical composition according to any embodiment described herein is administered for at least about 6 months. In some embodiments, a compound of Formula I, Formula II, and/or Formula III is administered for at least about 6 months.

In some embodiments, the therapeutically effective amount of a compound or pharmaceutical composition according to any embodiment described herein is from about 0.0001 mg to about 2000 mg, about 0.0001 mg to about 1500 mg, about 0.0001 mg to about 1200 mg, about 0.0001 mg to about 1000 mg, about 0.0001 mg to about 800 mg, about 0.0001 mg to about 500 mg, about 0.0001 mg to about 250 mg, about 0.0001 mg to about 200 mg, or about 0.0001 mg to about 100 mg. In some embodiments, the therapeutically effective amount of a compound or pharmaceutical composition according to any embodiment described herein is about 100 mg to about 300 mg. In some embodiments, the therapeutically effective amount is about 1 mg, about 5 mg, about 10 mg, about 25 mg, about 30 mg, about 50 mg, about 90 mg, about 100 mg, about 180 mg, about 280 mg, about 300 mg, about 450 mg, about 560 mg, about 840 mg, about 1120 mg, about 1500 mg, about 2000 mg.

In some embodiments, the methods described herein may be useful in qualifying Alzheimer's disease status in a patient that does not yet exhibit clinical symptoms of Alzheimer's disease.

Methods of the invention may further comprise reporting the status to the subject, recording the status on a tangible medium, and/or managing subject treatment based on the status.

### Measurement of Biomarkers

A variety of biological samples may be employed in any of the methods of the invention as described herein, including e.g. where the biological sample comprises blood or a blood derivative such as plasma, or where the biological sample comprises cerebrospinal fluid. In some embodiments, the biological sample is plasma, cerebrospinal fluid or a combination thereof.

Any biomarker according to any embodiment described herein, can be detected by any suitable method. Detection paradigms include optical methods, electrochemical methods (voltammetry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

Any biomarker according to any embodiment described herein can be measured by Liquid chromatography-mass spectrometry. In some embodiment, one or more biomarkers disclosed herein may be measured by mass spectrometry. The mass spectrometry may be SELDI-MS. In a further aspect, one or more biomarkers may be measured by immunoassay. In some embodiments, the immunoassay is an ELISA assay.

In one embodiment, a sample is analyzed by means of a biochip. A biochip generally comprises a solid substrate having a substantially planar surface, to which a capture reagent (also called an adsorbent or affinity reagent) is attached. Frequently, the surface of a biochip comprises a plurality of addressable locations, each of which has the capture reagent bound there. Protein biochips are biochips adapted for the capture of polypeptides. Many protein biochips are described in the art.

In some embodiments, the biomarkers disclosed herein are detected by mass spectrometry, a method that employs a mass spectrometer to detect gas phase ions. Examples of mass spectrometers are time-of-flight, magnetic sector, quadrupole filter, ion trap, ion cyclotron resonance, electrostatic sector analyzer and hybrids of these. In some embodiments, the biomarkers disclosed herein may be measured by Liquid chromatography-mass spectrometry.

In some embodiments, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer. The analysis of proteins by LDI can take the form of MALDI or of SELDI

According to any embodiment described herein, the biomarkers of the invention are measured by a method other than mass spectrometry or other than methods that rely on a measurement of the mass of the biomarker. In one such embodiment that does not rely on mass, the biomarkers of this invention are measured by immunoassay. Immunoassay requires biospecific capture reagents, such as antibodies, to capture the biomarkers. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well known in the art.

This invention contemplates traditional immunoassays including, for example, sandwich immunoassays including ELISA or fluorescence-based immunoassays, as well as other enzyme immunoassays. Nephelometry is an assay done in liquid phase, in which antibodies are in solution. Binding of the antigen to the antibody results in changes in absorbance, which is measured. In the SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry.

According to any embodiment described herein, one or more biomarkers can be measured by Liquid chromatography-mass spectrometry. In some embodiment, one or more biomarkers disclosed herein may be measured by mass spectrometry. The mass spectrometry suitably may be SELDI-MS. In a further aspect, one or more biomarkers may be measured by immunoassay. In some embodiments, the immunoassay is an ELISA assay.

According to any embodiment described herein, a sample can be analyzed by means of a biochip. A biochip generally comprises a solid substrate having a substantially planar surface, to which a capture reagent (also called an adsorbent or affinity reagent) is attached. Frequently, the surface of a biochip comprises a plurality of addressable locations, each of which has the capture reagent bound there. Protein biochips are biochips adapted for the capture of polypeptides. Many protein biochips are described in the art.

Any biomarkers according to any embodiment disclosed herein can be used in diagnostic tests to assess Alzheimer's disease status in a subject, e.g., to diagnose Alzheimer's disease. The phrase “Alzheimer's disease status” includes any distinguishable manifestation of the disease, including non-disease. For example, Alzheimer's disease status includes, without limitation, the presence or absence of disease (e.g., Alzheimer's disease v. non-Alzheimer's disease), the risk of developing disease, the stage of the disease, the progression of disease (e.g., progress of disease or remission of disease over time) and the effectiveness or response to treatment of disease.

The correlation of test results with Alzheimer's disease status may involve applying a classification algorithm of some kind to the results to generate the status. The classification algorithm may be as simple as determining whether or not the amount of biomarker measured is above or below a particular cut-off number or baseline measurement. When multiple biomarkers are used, the classification algorithm may be a linear regression formula. Alternatively, the classification algorithm may be the product of any of a number of learning algorithms described herein.

In the case of complex classification algorithms, it may be necessary to perform the algorithm on the data, thereby determining the classification, using a computer, e.g., a programmable digital computer. In either case, one can then record the status on tangible medium, for example, in computer-readable format such as a memory drive or disk or simply printed on paper. The result also could be reported on a computer screen.

### Determining Disease Status in a Subject

This invention provides methods for determining the presence or absence of Alzheimer's disease in a subject (status: Alzheimer's disease v. non-Alzheimer's disease). The presence or absence of Alzheimer's disease is determined by measuring the relevant biomarker or biomarkers and then either submitting them to a classification algorithm or comparing them with a reference amount and/or pattern of biomarkers that is associated with the particular risk level. In some embodiments, the presence or absence of Alzheimer's disease in a subject can be determined prior to the manifestation of any clinical symptoms indicative of the presence of Alzheimer's disease in a subject.

In one embodiment, this invention provides methods for determining the risk of developing Alzheimer's disease in a subject. In some embodiments, the subject does not exhibit clinical symptoms of Alzheimer's disease. The risk of developing a disease is determined by measuring the relevant biomarker or biomarkers and then either submitting them to a classification algorithm or comparing them with a reference amount and/or pattern of biomarkers that is associated with the Alzheimer's disease. In some embodiments, an increase in the expression of at least one biomarker selected from the group consisting of Chitinase 3 Like 1 (CH13L1), SPARC-Related Modular Calcium-Binding Protein 1 (SMOC 1), Clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), or any combination thereof may be indicative that a subject is at risk of developing Alzheimer's disease. In some embodiments, a change in the expression of at least one biomarker selected from the group consisting of Chitinase 3 Like 1 (CHI3L1), SPARC-Related Modular Calcium-Binding Protein 1 (SMOC 1), Clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and any combination thereof, may be indicative that a subject is at risk of developing Alzheimer's disease. In some embodiments, a decrease in the expression of at least one biomarker selected from the group consisting of chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and any combination thereof may be indicative of a low risk of developing Alzheimer's disease. Some embodiments are directed to methods for determining the stage of disease in a subject. Each stage of the disease has a characteristic amount of a biomarker or relative amounts of a set of biomarkers (a pattern). The stage of a disease is determined by measuring the relevant biomarker or biomarkers and then either submitting them to a classification algorithm or comparing them with a reference amount and/or pattern of biomarkers that is associated with the particular stage. For example, one can classify between mild, moderate and severe Alzheimer's disease.

In one embodiment, this invention provides methods for determining the course of disease in a subject. Disease course refers to changes in disease status over time, including disease progression (worsening) and disease regression (improvement). Over time, the amount or relative amounts (e.g., the pattern) of the biomarkers change. Accordingly, this method involves measuring one or more biomarkers in a subject for at least two different time points, e.g., a first time and a second time, and comparing the change in amounts, if any. The course of disease is determined based on these comparisons. In some embodiments, an increase in the expression of at least one biomarker selected from the group consisting of Chitinase 3 Like 1 (CHI3L1), SPARC-Related Modular Calcium-Binding Protein 1 (SMOC 1), Clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), or any combination thereof is indicative of disease progression. In some embodiments, a decrease in the expression of at least one biomarker selected from the group consisting of chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), or any combination thereof, may be indicative of a lack of disease progression. In some embodiments, a change in the expression of at least one biomarker selected from the group consisting of Chitinase 3 Like 1 (CHI3L1), SPARC-Related Modular Calcium-Binding Protein 1 (SMOC 1), Clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and any combination thereof, may be indicative of disease regression. In some embodiments, a decrease in the expression of at least one biomarker selected from the group consisting of Chitinase 3 Like 1 (CHI3L1), SPARC-Related Modular Calcium-Binding Protein 1 (SMOC 1), Clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and any combination thereof may be indicative of disease regression.

Additional embodiments of the invention relate to the communication of assay results or diagnoses or both to technicians, physicians or patients, for example. In certain embodiments, computers will be used to communicate assay results or diagnoses or both to interested parties, e.g., physicians and their patients. In some embodiments, the assays will be performed or the assay results analyzed in a country or jurisdiction which differs from the country or jurisdiction to which the results or diagnoses are communicated.

In certain embodiments of the methods of qualifying Alzheimer's disease status, the methods further comprise managing subject treatment based on the status. Such management includes the actions of the physician or clinician subsequent to determining Alzheimer's disease status. For example, if a physician makes a diagnosis of Alzheimer's disease, then a certain regime of treatment, such as prescription or administration of therapy might follow.

Alternatively, a diagnosis of non-Alzheimer's disease might be followed with further testing to determine a specific disease that the patient might be suffering from. Also, if the diagnostic test gives an inconclusive result on Alzheimer's disease status, further tests may be called for.

In some embodiments, data derived from the spectra (e.g., mass spectra or time-of-flight spectra) that are generated using samples such as “known samples” can then be used to “train” a classification model. A “known sample” is a sample that has been pre-classified. The data that are derived from the spectra and are used to form the classification model can be referred to as a “training data set.” Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased versus non-diseased).

Classification models can be formed using any suitable statistical classification (or “learning”) method that attempts to segregate bodies of data into classes based on objective parameters present in the data.

In another embodiment, this invention provides methods for determining the therapeutic efficacy of a pharmaceutical drug. These methods are useful in performing clinical trials of the drug, as well as monitoring the progress of a patient on the drug. Therapy or clinical trials involve administering the drug in a particular regimen. The regimen may involve a single dose of the drug or multiple doses of the drug over time. The doctor or clinical researcher monitors the effect of the drug on the patient or subject over the course of administration. If the drug has a pharmacological impact on the condition, the amounts or relative amounts (e.g., the pattern or profile) of the biomarkers of this invention changes toward a non-disease profile. Therefore, one can follow the course of the amounts of these biomarkers in the subject during the course of treatment. Accordingly, this method involves measuring one or more biomarkers in a subject receiving drug therapy, and correlating the amounts of the biomarkers with the disease status of the subject. One embodiment of this method involves determining the levels of the biomarkers for at least two different time points during a course of drug therapy, e.g., a first time and a second time, and comparing the change in amounts of the biomarkers, if any. For example, the biomarkers can be measured before and after drug administration or at two different time points during drug administration. The effect of therapy is determined based on these comparisons. If a treatment is effective, then the biomarkers will trend toward normal, while if treatment is ineffective, the biomarkers will trend toward disease indications. In some embodiments, the biomarker may be selected from the group consisting of Chitinase 3 Like 1 (CHJ3L1), SPARC-Related Modular Calcium-Binding Protein 1 (SMOC 1), Clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and any combination thereof. In some embodiments, the biomarker combinations may be made up of at least two biomarkers selected from the group consisting of Chitinase 3 Like 1 (CHI3L1), SPARC-Related Modular Calcium-Binding Protein 1 (SMOC 1), Clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and any combination thereof. In some embodiments, a change in protein expression that is inverse to a protein expression pattern typically observed in a subject with Alzheimer's disease compared with a healthy subject is indicative of therapeutic efficacy. In some embodiments, a decrease in the expression of at least one biomarker selected from the group consisting of chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and any combination thereof, is indicative of therapeutic efficacy. In some embodiments, a decrease in the expression of at least one biomarker, an increase in the expression of at least one biomarker, or a combination thereof may be indicative of therapeutic efficacy.

The methods of the present invention have other applications as well. For example, the biomarkers can be used to screen for compounds that modulate the expression of the biomarkers in vitro or in vivo, which compounds in turn may be useful in treating or preventing Alzheimer's disease in patients. In another example, the biomarkers can be used to monitor the response to treatments for Alzheimer's disease. In yet another example, the biomarkers can be used in heredity studies to determine if the subject is at risk for developing Alzheimer's disease.

Compounds suitable for therapeutic testing may be screened initially by identifying compounds which modulate the expression of at least one biomarker selected from the group consisting of chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and any combination thereof.

In some embodiments, the ability of a test compound to modulate the expression and/or the activity of one or more of the biomarkers selected from the group consisting of chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and any combination thereof, may be measured. One of skill in the art will recognize that the techniques used to measure the activity of a particular biomarker will vary depending on the function and properties of the biomarker. For example, an enzymatic activity of a biomarker may be assayed provided that an appropriate substrate is available and provided that the concentration of the substrate or the appearance of the reaction product is readily measurable. The ability of potentially therapeutic test compounds to inhibit or enhance the activity of a given biomarker may be determined by measuring the rates of catalysis in the presence or absence of the test compounds. The ability of a test compound to interfere with a non-enzymatic (e.g., structural) function or activity of one or more of the biomarkers herein may also be measured. For example, the self-assembly of a multi-protein complex which includes one or more of the biomarkers herein may be monitored by spectroscopy in the presence or absence of a test compound. Alternatively, if the biomarker is a non-enzymatic enhancer of transcription, test compounds which interfere with the ability of the biomarker to enhance transcription may be identified by measuring the levels of biomarker-dependent transcription in vivo or in vitro in the presence and absence of the test compound.

Test compounds capable of modulating the expression and/or activity of any of the biomarkers selected from the group consisting of chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and any combination there of may be administered to patients who are suffering from or are at risk of developing Alzheimer's disease.

Some embodiments are directed to methods for identifying compounds useful for the treatment of disorders such as Alzheimer's disease which are associated with changes in the expression of at least one biomarker selected from the group consisting of chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and any combination thereof. In some embodiments, the biomarker combinations may be made up of at least two biomarkers selected from the group consisting of chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and any combination thereof. In some embodiments, a useful therapeutic agent is an agent that when administered results in a change in biomarker expression that is inverse to a change in biomarker expression typically observed in a subject with Alzheimer's disease compared with a healthy subject.

In some embodiments, a useful therapeutic agent is an agent that when administered results in a decrease in the expression of at least one biomarker selected from the group consisting of chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and any combination thereof. In some embodiments, a useful therapeutic agent is an agent that when administered result in a decrease in the expression of at least one biomarker, an increase in the expression of at least one biomarker, or a combination thereof.

In some embodiments, screening a test compound includes obtaining samples from test subjects before and after the subjects have been exposed to a test compound. The levels in the samples of one or more of the biomarkers selected from the group consisting of chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and any combination thereof may be measured and analyzed to determine whether the levels of the biomarkers change after exposure to a test compound. The samples may be analyzed by mass spectrometry, as described herein, or the samples may be analyzed by any appropriate means known to one of skill in the art. For example, the levels of one or more of the biomarkers selected from the group consisting of chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and any combination thereof may be measured directly by Western blot using radio- or fluorescently-labeled antibodies which specifically bind to the biomarkers. Alternatively, changes in the levels of mRNA encoding the one or more biomarkers may be measured and correlated with the administration of a given test compound to a subject. In a further embodiment, the changes in the level of expression of one or more of the biomarkers may be measured using in vitro methods and materials. For example, human tissue cultured cells which express, or are capable of expressing, one or more of the biomarkers selected from the group consisting of chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and any combination thereof may be contacted with test compounds. Subjects who have been treated with test compounds will be routinely examined for any physiological effects which may result from the treatment. In particular, the test compounds will be evaluated for their ability to decrease disease likelihood in a subject. Alternatively, if the test compounds are administered to subjects who have previously been diagnosed with Alzheimer's disease, test compounds will be screened for their ability to slow or stop the progression of the disease.

Some embodiments are directed to qualifying Alzheimer's disease status in a subject comprising: (a) measuring the level of at least one biomarker selected from the group consisting of chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and any combination thereof in a biological sample from the subject being screened for Alzheimer's disease; and (b) correlating the measurement of an increased level of, chitinase 3 like 1 (CHI3L1), SPARC-related modular calcium-binding protein 1 (SMOC 1), clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), or any combination thereof in a biological sample from a healthy subject. In some embodiments, the at least one biomarker is measured by mass spectrometry. In some embodiments, the mass spectrometry is SELDI-MS. In some embodiments, the level of at the at least one biomarker is measured by immunoassay. In some embodiments, the sample is blood or a blood derivative. In some embodiments, the blood derivative is serum. In some embodiments, the sample is cerebrospinal fluid. In some embodiments, the correlating is performed by executing a software classification algorithm. Some embodiments further comprise (c) reporting the status to the subject. Some embodiments further comprising: recording the status on a tangible medium. Some embodiments further comprise (c) managing subject treatment based on the status. Some embodiments further comprise: (d) measuring the level of the at least one biomarker after subject management and correlating the measurement with disease progression. In some embodiments, the subject does not exhibit clinical symptoms of Alzheimer's disease.

### EXAMPLES

### Example 1: CT1812 Meets Phase 1b/2a Study Objectives: Well Tolerated with Positive Influence on Biomarkers of Synapse Recovery

CT1812, is an orally-administered lipophilic isoindoline as a fumarate; that is rapidly absorbed, highly brain penetrant. CT1812 is being developed for the treatment of mild-to-moderate Alzheimer's disease, is a highly brain penetrant small molecule that displaces amyloid beta (AD) oligomers from neuronal receptors, potentially allowing synapses to regenerate and cognitive performance to improve. Early clinical development has shown that CT1812 is safe and well tolerated at single doses of up to 1120 mg up to 840 mg in young and 560 mg in elderly (aged 65-75) with 14-day multiple dose (QD). Drug interaction studies showed minor interactions with CYP isoenzymes.

This study enrolled a total of 19 individuals with mild-to-moderate Alzheimer's disease who were randomized to receive placebo or one of three doses of CT1812: 90 mg, 280 mg and 560 mg for 28 days. Safety and pharmacokinetics were primary objectives of the study, with changes in molecular biomarkers and cognitive outcomes identified as an exploratory objective.

All three doses were well tolerated with no drug-related significant adverse events reported. All adverse events were mild or moderate and resolved by the end of study. Pharmacokinetics were consistent with previous clinical study results, and suggested that CT1812 achieved greater than 80 percent receptor occupancy at all doses, a level previously demonstrated to be the minimum efficacious concentration. Cognitive outcomes were similar across the treatment groups.

Notably, treatment with CT1812 resulted in a differential change compared to placebo in the levels of 30 proteins, several of which play key roles in synaptic plasticity and are dysregulated in Alzheimer's disease. Among these is neurogranin, a synaptic protein that has been indicated as a predictor of cognitive decline when elevated. In an analysis of pooled CT1812-treated patients compared to those receiving placebo, the reduction in the level of neurogranin was statistically significant (p=0.05). In addition, treatment with CT1812 resulted in a reduction in synaptotagmin-1, a synaptic biomarker that is elevated in the cerebral spinal fluid of patients with Alzheimer's disease.

The study results yielded some meaningful insights about the activity of CT1812 in patients with mild to moderate Alzheimer's disease. The changes observed in biomarker levels after treatment with CT1812 were consistent with a positive effect on synapses.

### Example 2: Biomarker Outcomes from the Phase 1b/2a Safety Trial of the Anti-Aβ Oligomer Drug CT1812 in Alzheimer's Patients

### Abstract

Background: CT1812 is the only therapeutic candidate demonstrated to displace Aβ oligomers from synaptic receptor sites and clear them from the brain into the cerebrospinal fluid, restoring normal cognitive performance in aged transgenic mouse models of AD.

Chronic treatment of aged transgenic mice with efficacious doses of CT1812 significantly reduces inflammatory protein expression in CSF, and normalizes Alzheimer's disease-related protein expression in CSF and plasma as measured by LC/MSMS. CT1812 appears safe and well tolerated with multiple doses up to 560 mg/day in healthy elderly volunteers (ClinicalTrials.gov NCT02570997). To further the clinical development of CT1812, we completed a clinical trial in mild to moderate Alzheimer's patients to evaluate protein biomarkers as well as safety (ClinicalTrials.gov NCT02907567).

Methods: A multi-center, double-blind, placebo-controlled parallel group trial was performed to evaluate the safety, tolerability and pharmacokinetics of three doses of CT1812 (90, 280 and 560 mg) or placebo (N=4 or 5 patients/group) given once daily for 28 days to Alzheimer's patients (MMSE 18-26). Plasma and CSF protein expression were measured by LC/MSMS in samples drawn prior to dosing (Day 0) and at end of dosing (Day 28) and were compared within each patient and between dosing groups.

Results: LC/MSMS analysis resulted in the identification and relative quantitation of 911 CSF proteins and 1965 plasma proteins across all subjects. Changes in expression of specific proteins were observed in both CSF and plasma following treatment with study drug. Multiple proteins were upregulated in the CSF in response to drug. These include proteins previously linked to Alzheimer's disease and proteins involved in axon guidance/CNS development, all of which could be expected to increase with disease reversion. The relationship between protein function and disease, and association with therapeutic target receptor pathways will be reported in detail, along with additional CSF outcomes including Aβ 40, 42 tau and p-tau).

Conclusions: Treatment of Alzheimer's patients with study drug once daily for 28 days results in protein expression changes in plasma and CSF as measured by LC/MSMS. Along with safety and clinical outcomes, the protein expression outcomes will help guide the future development of CT1812. Additional trials include an indwelling lumbar catheter study to detect changes in Aβ oligomers in CSF, a PET study to assess synaptic density after treatment with CT1812 and a Phase 2 six-month efficacy trial.

### Methods

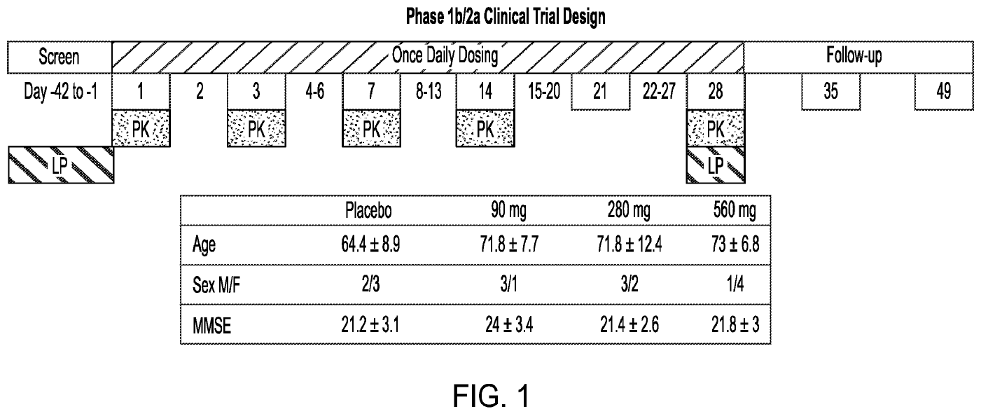

COG0102 clinical trial: This trial (ClinicalTrials.gov NCT02907567) was a randomized, double-blind, placebo-controlled clinical trial in mild to moderate Alzheimer's patients (MMSE 18-26) (See FIG. 1 for clinical trial design). The trial was conducted at six sites in Australia. Patients were randomized into placebo (n=5) or CT1812-treated groups: 90 mg (n=4), 280 mg (n=5) or 560 mg (n=5) and treated once daily for 28 days. Clinical chemistry was done at Sydpath (Sydney, AU). Pharmacokinetics were done at CPR Pharma Services (Adelaide, AU). Cerebrospinal fluid samples were taken at screening and day 21-28 of treatment and aliquoted. Plasma samples were taken at screening or Day 1 (one sample) and day 14-28, then spun down to separate plasma and aliquoted. One set of CSF samples was analyzed for Alzheimer's disease biomarkers; protein measurements (Aβ42, Aβ40, total tau, tau phosphorylated at threonine 181 (phosphorylated tau), NFL and neurogranin) were performed using commercially available ELISA assays from Fujirebio according to the manufacturer's instructions (INNOTEST β-AMYLOID(1-42), INNOTEST β-AMYLOID(1-40), INNOTEST hTau Ag, INNOTEST PHOSPHO-TAU(181P), Uman Diagnostics NFL assay). Neurogranin was measured with a sandwich ELISA method using antibodies Ng2 and Ng22 developed at the University of Gotteborg (Kvartsberg et al. 2015). Identical aliquots of CSF, and plasma were analyzed for protein expression changes via LC/MSMS at Caprion (see below).

TABLE 2
Phase 1b/2a clinical study parameters

| | |
|---|---|
| Population | 19 participants, 50-80 years, mild/ |
| | moderateAlzheimer's disease MMSE 18-26 |
| N Per Dose Group | Placebo (n = 5), 90 mg (n = 4), 280 mg (n = 5), |
| | 560 mg (n = 5) |
| Dosing | 1x daily for 28 days |
| Primary Objectives | Safety and tolerability |
| Secondary Objectives | Pharmacokinetics |
| Exploratory Objectives | ADAS-Cog, COWAT, CFT, and composite |
| | CSF concentrations of CT1812 and CSF biomarkers |

Neurogranin expression study in vitro: 0.5 uM synthetic Aβ oligomers were added to DIV21 hippocampal/cortical cultures (described in Izzo et al., 2014 a,b) for 1 hour prior to 4.8 nM of CT1812, then incubated for 24 hours. Neurons were fixed in 3.75% formaldehyde and stained with full-length neurogranin (Abcam catalogue #ab23570, used in combination with novel Ng7 to detect full length neurogranin inAlzheimer's disease patient brain IP; Kvartsberg et al. 2015 Blennow lab), MAP2 (Millipore), and 4G8 (Biolegend) antibodies. Imaging was performed on Cellomics VX automated microscope with a 20×, 0.75 NA objective and analyzed using a compartmental analysis algorithm to measure staining in the nucleus versus cytoplasm.

LC MSMS biomarker discovery: Matched screening and day 28 plasma and CSF samples were analyzed by LC/MSMS at Caprion Biosciences Inc. Plasma was depleted of high and medium abundance proteins using a commercial immunoaffinity column, IgY14-Supermix (Sigma). CSF was depleted of only 14 high abundance proteins using MARS-14 (Agilent). All samples were digested with trypsin and plasma samples were fractionated by strong cation exchange chromatography (3 fractions). Both CSF and plasma samples were analyzed using a NanoAcquity UPLC coupled to a Q Exactive MS. Peptide separation was achieved with a nanoAcquity Symmetry UPLC Trap column and nanoAcquity UPLC BEH300 analytical column. The 12 most intense peaks per survey scan with charge states 2-8 fragmented and scanned with a mass range from 200 to 2000 m/z at a resolution of 17,500. Raw spectrometer data files for each LC-MS run were aligned independently using Elucidator software (Rosetta Biosoftware). The MS/MS spectra were matched to corresponding peptide sequences found in the Uniprot Human protein database (January 2017) using Mascot software, allowing for up to 2 missed cleavages, a peptide tolerance of 20 ppm, and an MS/MS tolerance of 0.05 Da. Outlier detection was performed by determining the average log-intensity of all isotope groups (IG) over injection order for all samples. Samples with an average value greater than 2 standard deviations from the mean were flagged for investigation. Following data transformation and normalization, expression analysis of the identified isotope groups was performed and the statistical significance of each comparison was assessed with a parametric, linear mixed model (LMM) and a non-parametric Wilcoxon ranked test (ranked p-values). Expression analysis was also performed at the peptide and protein levels, which used the same methodology as above, but applied to peptide/protein intensities, derived by rolling-up the corresponding isotope group intensities. Isotope groups not detected in at least half of the samples in either of the two groups being compared were not used for the roll-up.

Heat map analysis: The difference in protein abundance between day 28 and baseline for each sample was normalized for graphical comparison by converting to z-score. First, the mean and standard deviation (SD) of day 28—baseline values were calculated across all paired samples for each protein. Then, for each patient's paired sample fold change, the difference from the mean was determined and divided by the SD to derive the z-score. These values, normalized by SD to the same scale, allow direct comparison of relative change from baseline in response to treatment for each protein in each subject.

### Results

The change in cognitive testing scores from baseline was similar across all groups in all tests, as might be expected in a study of only 28 days duration. CT1812 is generally safe and well tolerated at all doses. No severe AEs or SAEs were reported and all AEs were mild or moderate (See Table 3 and 4. One participant showed ALT˜4.7×ULN at 560 mg which resolved by the end of study and had no associated increase in bilirubin. Four patients had lymphocytopenia (3 mild) at 560 mg which resolved by the end of the study. Cognitive outcomes were similar across the treatment groups.

TABLE 3
Treatment-Emergent Adverse Events

| | Placebo | 90 mg | 280 mg | 560 mg |
|---|---|---|---|---|
| | (n = 5) | (n = 4) | (n = 5) | (n = 5) |
| TEAEs-n (%) | 3 (60%) | 3 (75%) | 4 (80%) | 5 (100%) |
| Mild TEAEs-n | 3 (60%) | 4 (100%) | 3 (60%) | 5 (100%) |
| (%) | | | | |
| Moderate TEAEs- | 1 (20%) | 0 (0%) | 1 (20%) | 3 (60%) |
| n (%) | | | | |
| Severe TEAEs-n | 0 (0%) | 0 (0%) | 0 (0%) | 0 (0%) |
| (%) | | | | |
| SAEs-n (%) | 0 (0%) | 0 (0%) | 0 (0%) | 0 (0%) |

TABLE 4
Adverse events occurring in more than 1 participant
Adverse Events Occurring in More Than 1 Participant

| | | | | |
|---|---|---|---|---|
| Lymphocytopenia- | 0 (0%) | 0 (0%) | 0 (0%) | 3 (60%)* |
| n (%) | | | | |
| Headache-n (%) | 0 (0%) | 1 (25%) | 0 (0%) | 2 (40%) |
| Nausea-n (%) | 1 (20%) | 0 (0%) | 0 (0%) | 2 (40%) |
| Vomiting-n (%) | 1 (20%) | 0 (0%) | 0 (0%) | 2 (40%) |

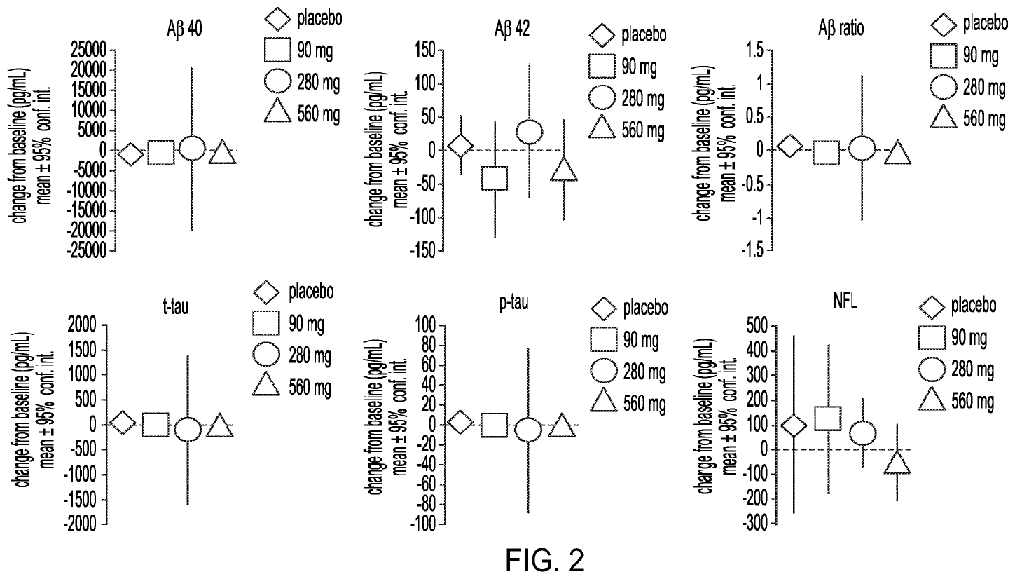

Most biomarker levels remain unchanged with CT1812 treatment. FIG. 2 depicts Aβ, tau, and NFL levels before and after treatment with CT812.

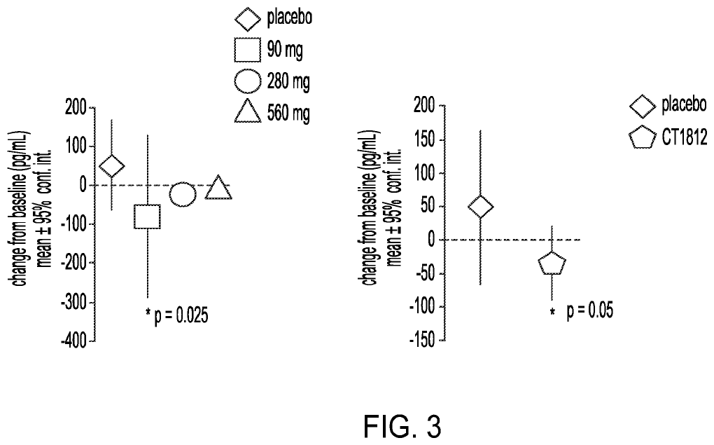

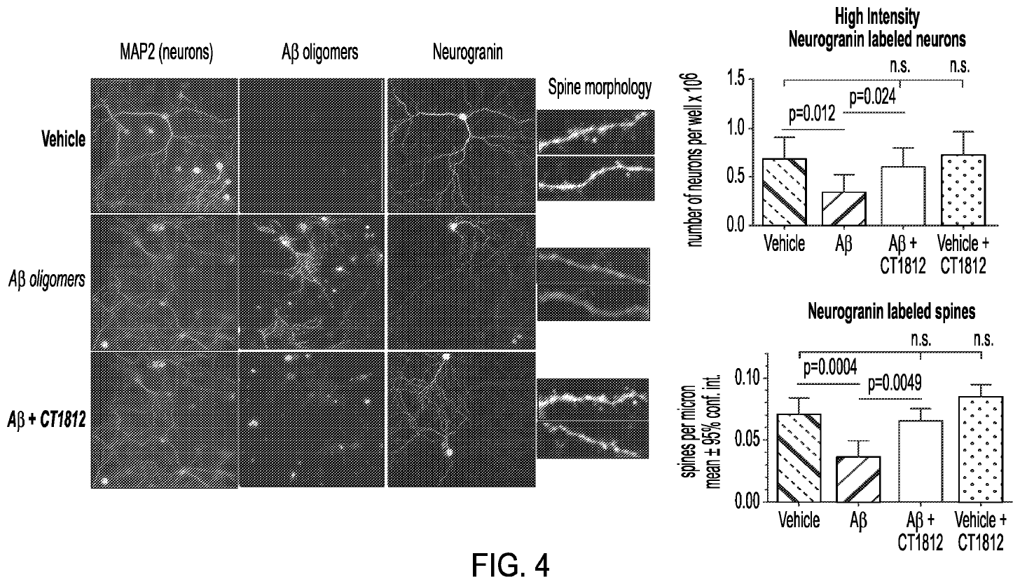

However, neurogranin, a synaptic damage marker elevated in Alzheimer's CSF was reduced by 33% in the 90 mg dose group and 17.6% in the pooled CT1812-treated group (See FIG. 3). Reduction was consistent with a positive effect on synapses, CT1812's mechanism of action, and preclinical studies. Neurogranin levels were decreased consistent with increase in cleaved, secreted forms of neurogranin in Alzheimer's CSF (see FIG. 4) where Aβ42 oligomer exposure restores neurogranin expression consistent with positive effect on synapses. Aβ42 oligomers were added to mature (21 days in vitro) hippocampal/cortical cultures (500 nM, 1 hr) followed by CT1812 (4.8 nM) or vehicle treatment (24 hr)).

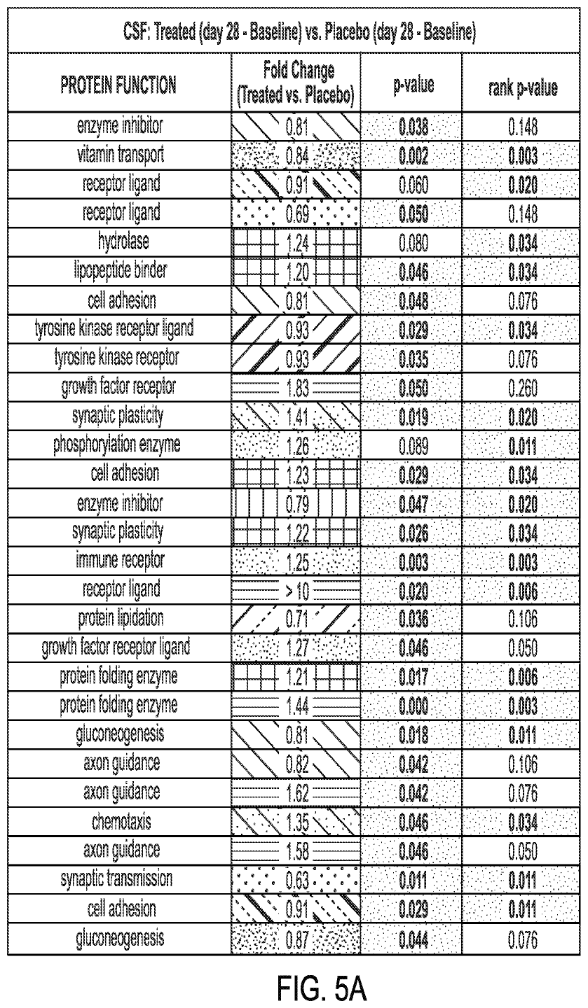

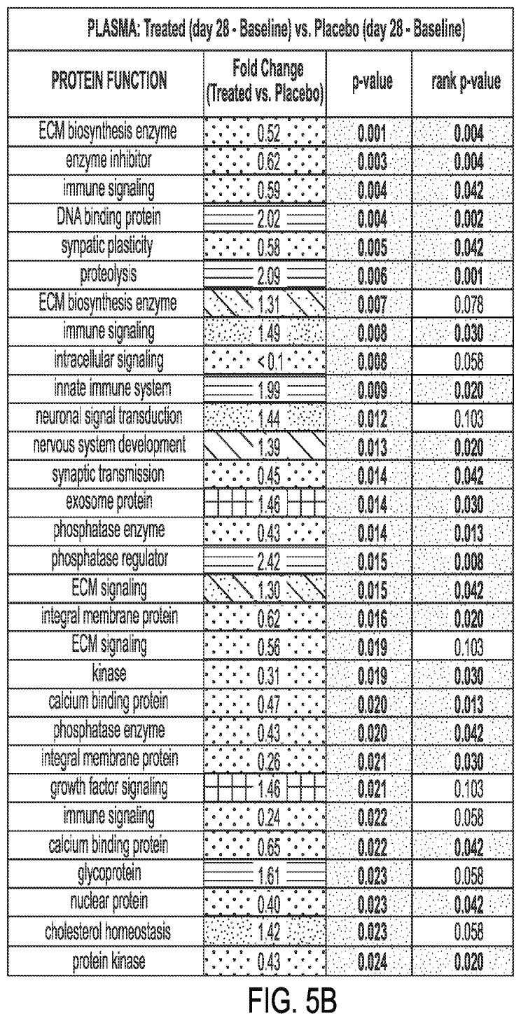

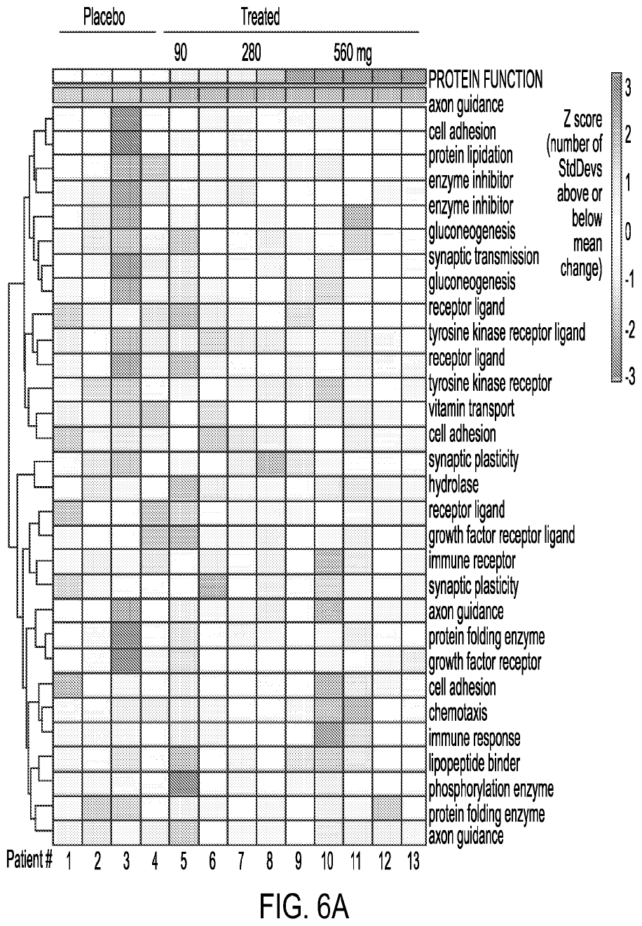

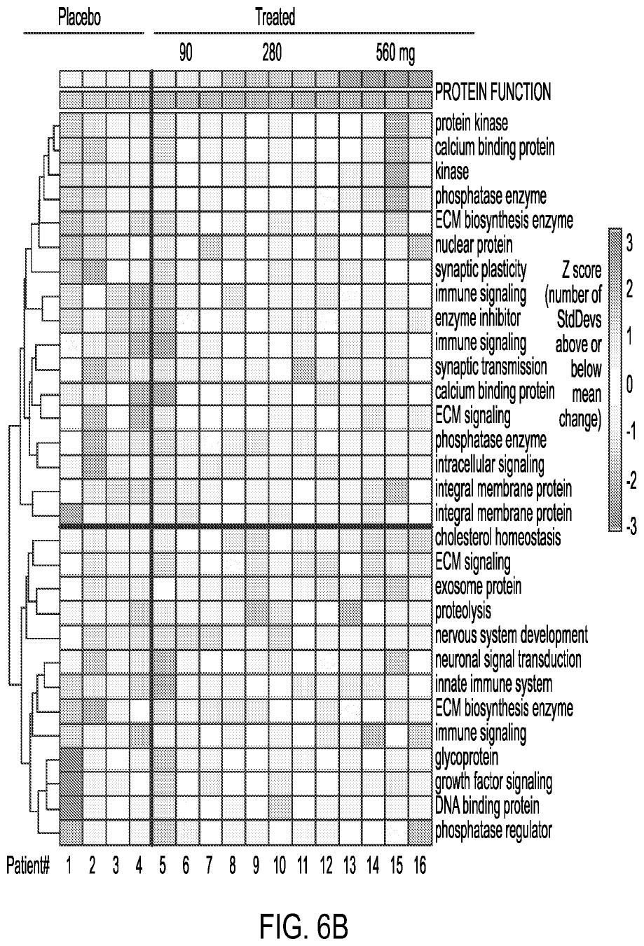

The concentrations of 30 CSF proteins changed differentially in the CT1812 treatment group versus placebo (p<0.05). FIG. 5 depicts various protein expression levels CT1812 for 28 days versus placebo in CSF and plasma. FIG. 6 depicts 30 proteins changed differentially in CT1812-treated vs. placebo patients (p<0.05, i.e., higher or lower expression vs. placebo). Several play key roles in synaptic plasticity and are dysregulated in Alzheimer's disease brain: Synaptotagmin-1, a synaptic damage marker elevated in Alzheimer's CSF. Expression decreased 63% in CT1812-treated vs. placebo consistent with positive effect on synapses and CT1812's mechanism of action.

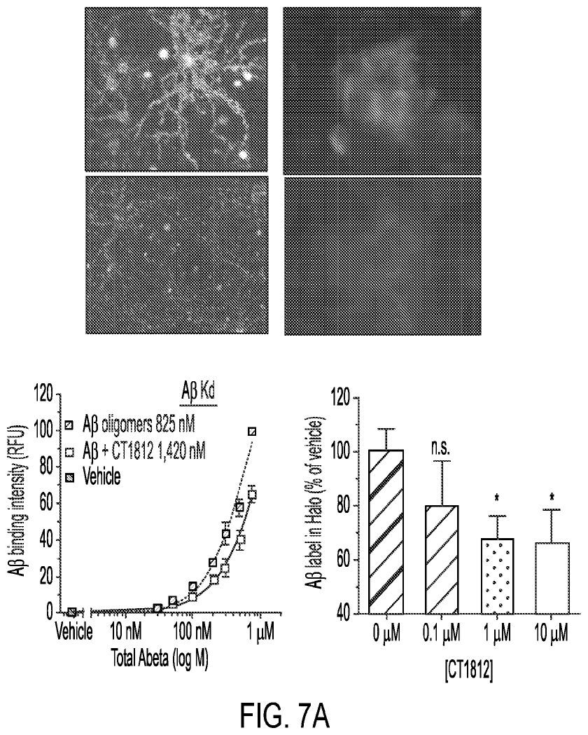

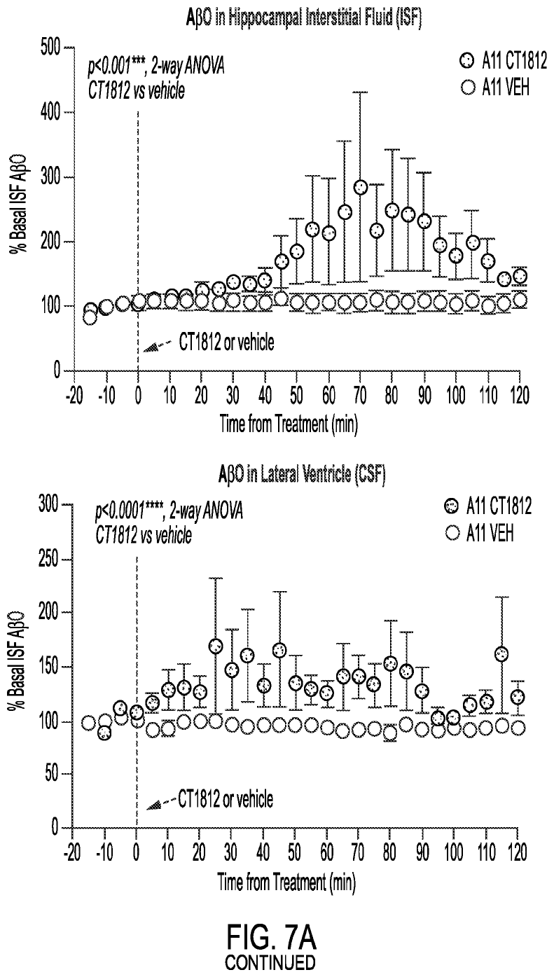

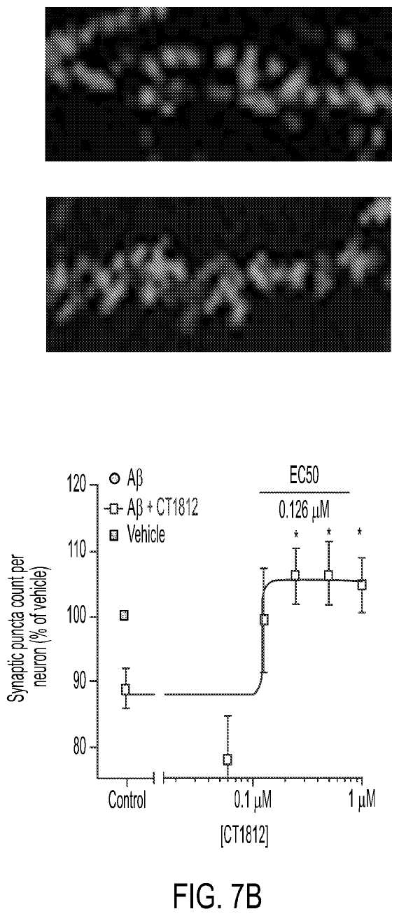

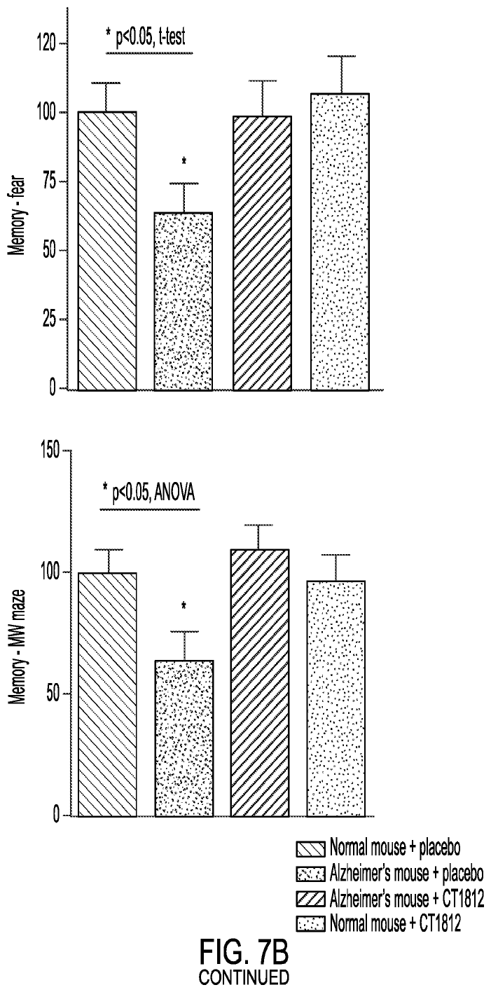

CT1812 is believed to be a Sigma-2/PGRMC1* receptor complex allosteric antagonist, destabilizes the Aβ oligomer binding site, increases off-rate of oligomers from synaptic receptors, Aβ oligomers then cleared into CSF. FIG. 7 depicts the displacement of Aβ oligomers from neurons, from Alzheimer's disease patient neurocortical tissue, from the hippocampus of living transgenic APP/PS1 mice (where Aβ oligos are cleared into the CSF without affecting monomer concentrations. Also shown is the restoration of synapse number and memory in transgenic Alzheimer's disease mice at concentrations of greater than 80% receptor occupancy.

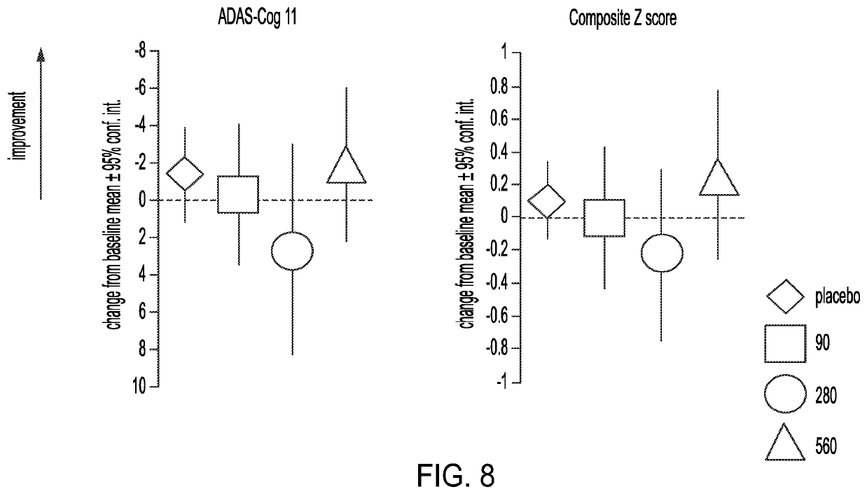

FIG. 8 depicts exploratory cognitive outcomes from the phase 1b/2a clinical trial. Changes from baseline were similar across groups.

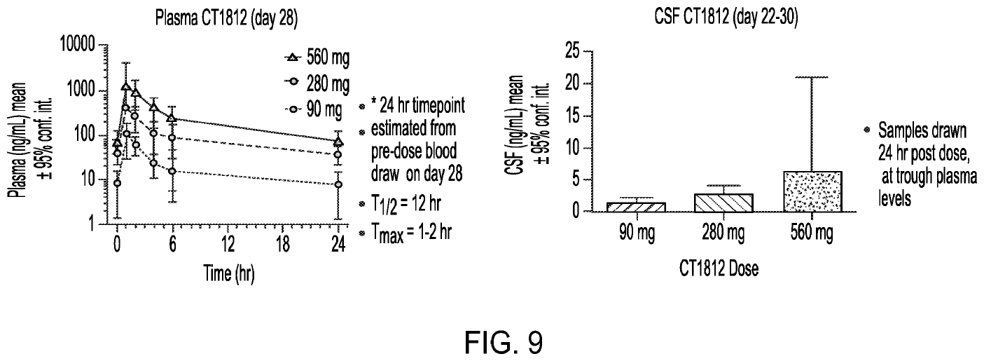

FIG. 9 depicts CT1812 pharmacokinetic parameters in the plasma (day 28) and CSF (days 22-30). Plasma CT1812 concentration increased approximately dose proportionally while a dose dependent increase in CSF concentration was observed. All CSF concentrations were greater than 80% estimated brain receptor occupancy (threshold needed to demonstrate efficacy in preclinical studies).

### Conclusions:

CT1812 is safe and well tolerated across all doses, no SAEs. Greater than 80% estimated brain receptor occupancy was achieved at all doses (threshold needed to demonstrate efficacy in preclinical studies). The concentrations of 30 CSF proteins changed differentially in the CT1812 treatment group versus placebo (p<0.05). CSF synaptic damage markers decreased (neurogranin and synaptotagmin-1), consistent with a positive synaptic effect and CT1812's mechanism of action

### Example 3—Proteomic Analysis of Cerebrospinal Fluid (CSF) from Clinical Trial Subjects

Table 5 lists 30 proteins that were detected with at least 20% change in placebo vs. treated 28 days vs screening. The following values are reported for each statistical analysis performed: FC: Fold change calculated from the protein normalized intensity. FC>10 or <0.1 are unusually large and may be artifacts. The color shading for the FC values is a function of the direction and amplitude of the observed fold-change. Values corresponding to up-regulation in the Group of interest are displayed in shades of red and those corresponding to down-regulation in shades of blue. p-value: Significance of the protein-level statistical test. p-values meeting statistical significance arbitrary threshold are colored in grey. Ranked p-value: Significance of the protein-level using Wilcoxon ranked statistical test. p-values meeting statistical significance arbitrary threshold are colored in grey.

TABLE 5
Changes in protein expression levels in CSF of trial subjects
treated with CT812 for 28 days versus placebo

| | | | FC | | | |
|---|---|---|---|---|---|---|
| | | | (Treated | | rank | |
| | FC | FC | vs. | p- | p- | |
| Gene | (Treated) | (Placebo) | Placebo) | value | value | Protein Description |
| PDIA6 | 1.15 | 0.80 | 1.44 | 0.000 | 0.003 | Protein disulfide-isomerase |
| | | | | | | A6 (EC 5.3.4.1) (Endoplasmic |
| | | | | | | reticulum protein 5) (ER |
| | | | | | | protcin 5) (ERp5) (Protcin |
| | | | | | | disulfide isomerase P5) |
| | | | | | | (Thioredoxin domain- |
| | | | | | | containing protein 7) |
| AFM | 0.94 | 1.12 | 0.84 | 0.002 | 0.003 | Afamin (Alpha-albumin) |
| | | | | | | (Alpha-Alb) |
| LILRB4 | 1.11 | 0.90 | 1.25 | 0.003 | 0.003 | Leukocyte immunoglobulin- |
| | | | | | | like receptor subfamily B |
| | | | | | | member 4 (CD85 antigen-like |
| | | | | | | family member K) |
| | | | | | | (Immunoglobulin-like |
| | | | | | | transcript 3) (ILT-3) |
| | | | | | | (Leukocyte immunoglobulin- |
| | | | | | | like receptor 5) (LIR-5) |
| | | | | | | (Monocyte inhibitory receptor |
| | | | | | | HM18) (CD antigen CD85k) |
| SYT1 | 0.78 | 1.24 | 0.63 | 0.011 | 0.011 | Synaptotagmin-1 |
| | | | | | | (Synaptotagmin I) (SytI) |
| | | | | | | (p65) |
| P4HB | 1.10 | 0.91 | 1.21 | 0.017 | 0.006 | Protein disulfide-isomerase |
| | | | | | | (PDI) (EC 5.3.4.1) (Cellular |
| | | | | | | thyroid hormone-binding |
| | | | | | | protein) (Prolyl 4-hydroxylase |
| | | | | | | subunit beta) (p55) |
| RBP4 | 0.82 | 1.02 | 0.81 | 0.018 | 0.011 | Retinol-binding protein 4 |
| | | | | | | (Plasma retinol-binding |
| | | | | | | protein) (PRBP) (RBP) |
| | | | | | | [Cleaved into: Plasma retinol- |
| | | | | | | binding protein(1-182); |
| | | | | | | Plasma retinol-binding |
| | | | | | | protein(1-181); Plasma |
| | | | | | | retinol-binding protein(1- |
| | | | | | | 179); Plasma retinol-binding |
| | | | | | | protein(1-176)] |
| GMFB | 1.04 | 0.74 | 1.41 | 0.019 | 0.020 | Glia maturation factor beta |
| | | | | | | (GMF-beta) |
| MBL2 | 0.75 | <0.1 | >10 | 0.020 | 0.006 | Mannose-binding protein C |
| | | | | | | (MBP-C) (Collectin-1) |
| | | | | | | (MBP1) (Mannan-binding |
| | | | | | | protein) (Mannose-binding |
| | | | | | | lectin) |
| CAMK2D | 1.04 | 0.85 | 1.22 | 0.026 | 0.034 | Calcium/calmodulin- |
| | | | | | | dependent protein kinase type |
| | | | | | | II subunit delta (CaM kinase |
| | | | | | | II subunit delta) (CaMK-II |
| | | | | | | subunit delta) (EC 2.7.11.17) |
| EFNB1 | 0.97 | 1.04 | 0.93 | 0.029 | 0.034 | Ephrin-B1 (EFL-3) (ELK |
| | | | | | | ligand) (ELK-L) (EPH-related |
| | | | | | | receptor tyrosine kinase |
| | | | | | | ligand 2) (LERK-2) |
| ICAM2 | 1.11 | 0.90 | 1.23 | 0.029 | 0.034 | Intercellular adhesion |
| | | | | | | molecule 2 (ICAM-2) (CD |
| | | | | | | antigen CD102) |
| TNC | 1.04 | 1.15 | 0.91 | 0.029 | 0.011 | Tenascin (TN) (Cytotactin) |
| | | | | | | (GMEM) (GP 150-225) |
| | | | | | | (Glioma-associated- |
| | | | | | | extracellular matrix antigen) |
| | | | | | | (Hexabrachion) (JI) |
| | | | | | | (Myotendinous antigen) |
| | | | | | | (Neuronectin) (Tenascin-C) |
| | | | | | | (TN-C) |
| EPHA4 | 0.94 | 1.01 | 0.93 | 0.035 | 0.076 | Ephrin type-A receptor 4 (EC |
| | | | | | | 2.7.10.1) (EPH-like kinase 8) |
| | | | | | | (EK8) (hEK8) (Tyrosine- |
| | | | | | | protein kinase TYRO1) |
| | | | | | | (Tyrosine-protein kinase |
| | | | | | | receptor SEK) |
| MDGA2 | 1.01 | 1.42 | 0.71 | 0.036 | 0.106 | MAM domain-containing |
| | | | | | | glycosylphosphatidylinositol |
| | | | | | | anchor protein 2 (MAM |
| | | | | | | domain-containing protein 1) |
| YWHAB | 0.90 | 1.11 | 0.81 | 0.038 | 0.148 | 14-3-3 protein beta/alpha |
| | | | | | | (Protein 1054) (Protein kinase |
| | | | | | | C inhibitor protein 1) (KCIP- |
| | | | | | | 1) [Cleaved into: 14-3-3 |
| | | | | | | protein beta/alpha, N- |
| | | | | | | terminally processed] |
| ROBO4 | 1.22 | 0.75 | 1.62 | 0.042 | 0.076 | Roundabout homolog 4 |
| | | | | | | (Magic roundabout) |
| ROBO1 | 0.96 | 1.18 | 0.82 | 0.042 | 0.106 | Roundabout homolog 1 |
| | | | | | | (Deleted in U twenty twenty) |
| | | | | | | (H-Robo-1) |
| TPII | 0.90 | 1.04 | 0.87 | 0.044 | 0.076 | Triosephosphate isomerase |
| | | | | | | (TIM) (EC 5.3.1.1) (Triose- |
| | | | | | | phosphate isomerase) |
| PDGFA | 1.15 | 0.91 | 1.27 | 0.046 | 0.050 | Platelet-derived growth factor |
| | | | | | | subunit A (PDGF subunit A) |
| | | | | | | (PDGF-1) (Platelet-derived |
| | | | | | | growth factor A chain) |
| | | | | | | (Platelet-derived growth |
| | | | | | | factor alpha polypeptide) |
| SAA4 | 1.17 | 0.87 | 1.35 | 0.046 | 0.034 | Serum amyloid A-4 protein |
| | | | | | | (Constitutively expressed |
| | | | | | | serum amyloid A protein) (C- |
| | | | | | | SAA) |
| SEMA3F | 1.40 | 0.89 | 1.58 | 0.046 | 0.050 | Semaphorin-3F (Sema III/F) |
| | | | | | | (Semaphorin IV) (Sema IV) |
| CD14 | 1.18 | 0.98 | 1.20 | 0.046 | 0.034 | Monocyte differentiation |
| | | | | | | antigen CD14 (Myeloid cell- |
| | | | | | | specific leucine-rich |
| | | | | | | glycoprotein) (CD antigen |
| | | | | | | CD14) [Cleaved into: |
| | | | | | | Monocyte differentiation |
| | | | | | | antigen CD14, urinary form; |
| | | | | | | Monocyte differentiation |
| | | | | | | antigen CD14, membrane- |
| | | | | | | bound form] |
| SERPINA4 | 0.88 | 1.12 | 0.79 | 0.047 | 0.020 | Kallistatin (Kallikrein |
| | | | | | | inhibitor) (Peptidase inhibitor |
| | | | | | | 4) (PI-4) (Serpin A4) |
| CNTN1 | 0.97 | 1.19 | 0.81 | 0.048 | 0.076 | Contactin-1 (Glycoprotein |
| | | | | | | gp135) (Neural cell surface |
| | | | | | | protein F3) |
| FGFR3 | 1.12 | 0.61 | 1.83 | 0.050 | 0.260 | Fibroblast growth factor |
| | | | | | | receptor 3 (FGFR-3) (EC |
| | | | | | | 2.7.10.1) (CD antigen CD333) |
| ANGPTL2 | 0.80 | 1.17 | 0.69 | 0.050 | 0.148 | Angiopoietin-related protein 2 |
| | | | | | | (Angiopoietin-like protein 2) |
| NPPC | 0.90 | 0.99 | 0.91 | 0.060 | 0.020 | C-type natriuretic peptide |
| | | | | | | [Cleaved into: CNP-22; CNP- |
| | | | | | | 29; CNP-53] |
| SMPDL3B | 0.97 | 0.79 | 1.24 | 0.080 | 0.034 | Acid sphingomyelinase-like |
| | | | | | | phosphodiesterase 3b (ASM- |
| | | | | | | like phosphodiesterase 3b) |
| | | | | | | (EC 3.1.4.—) |
| GNPTG | 1.18 | 0.94 | 1.26 | 0.089 | 0.011 | N-acetylglucosamine-1- |
| | | | | | | phosphotransferase subunit |
| | | | | | | gamma (GlcNAc-1- |
| | | | | | | phosphotransferase subunit |
| | | | | | | gamma) (UDP-N- |
| | | | | | | acetylglucosamine-1- |
| | | | | | | phosphotransferase subunit |
| | | | | | | gamma) |
| HLA-H | 1.22 | 0.89 | 1.37 | 0.169 | 0.034 | Putative HLA class I |
| | | | | | | histocompatibility antigen, |
| | | | | | | alpha chain H (HLA-12.4) |
| | | | | | | (HLA-AR) (MHC class I |
| | | | | | | antigen H) |

The compounds provided herein can be synthesized via any synthetic route; for example, see WO2013/029060, and WO2013/029067, each of which is incorporated herein by reference.

TABLE 6
Additional Isoindoline Compounds for use in the methods
describned herein.
Structure

| | |
|---|---|
| 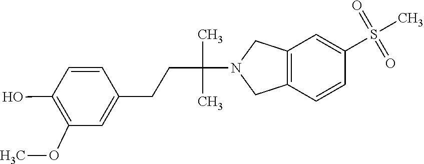 [SMILES: COc1cc(CCC(C)(C)N2Cc3ccc(S(C)(=O)=O)cc3C2)ccc1O] | Ex. Cpd. No. 77 |
| 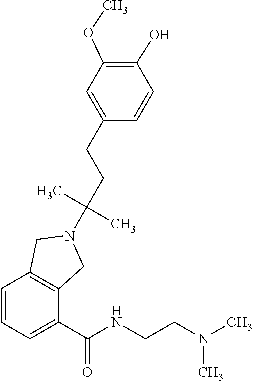 [SMILES: COc1cc(CCC(C)(C)N2Cc3cccc(C(=O)NCCN(C)C)c3C2)ccc1O] | Ex. Cpd. No. 78 |
| 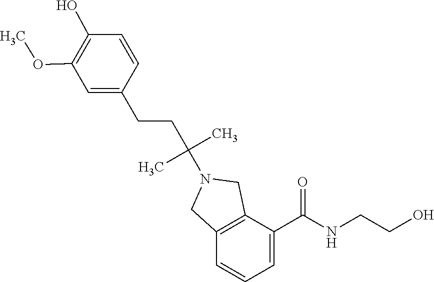 [SMILES: COc1cc(CCC(C)(C)N2Cc3cccc(C(=O)NCCO)c3C2)ccc1O] | Ex. Cpd. No. 79 |
| 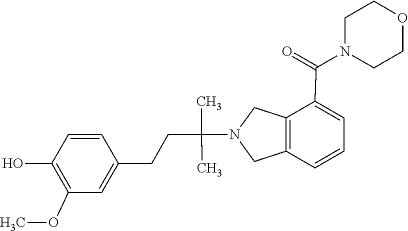 [SMILES: COc1cc(CCC(C)(C)N2Cc3cccc(C(=O)N4CCOCC4)c3C2)ccc1O] | Ex. Cpd. No. 80 |
| 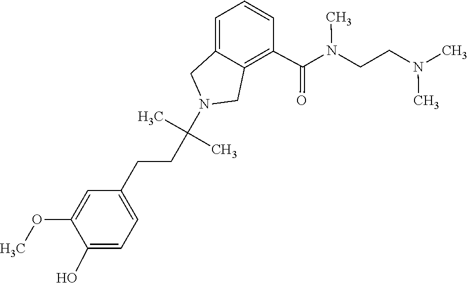 | Ex. Cpd. No. 81 |
| 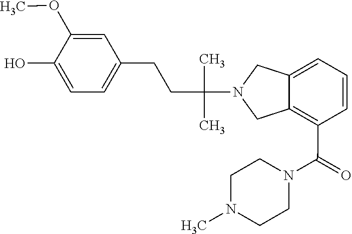 [SMILES: COc1cc(CCC(C)(C)N2Cc3cccc(C(=O)N4CCN(C)CC4)c3C2)ccc1O] | Ex. Cpd. No. 82 |
| 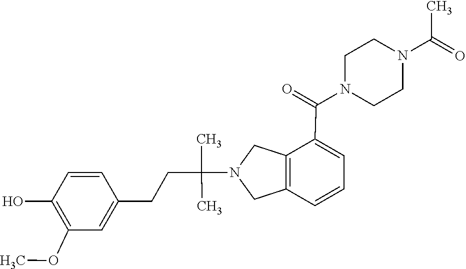 [SMILES: COc1cc(CCC(C)(C)N2Cc3cccc(C(=O)N4CCN(C(C)=O)CC4)c3C2)ccc1O] | Ex. Cpd. No. 83 |
| 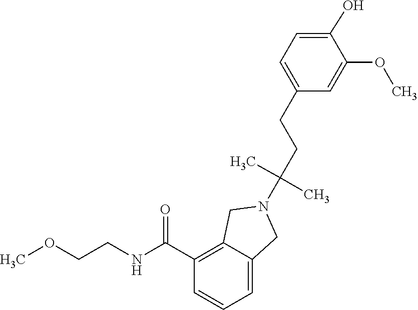 | Ex. Cpd. No. 84 |
| 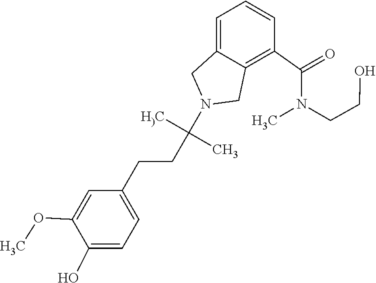 | Ex. Cpd. No. 85 |
| 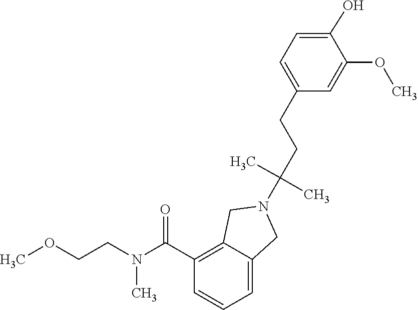 | Ex. Cpd. No. 86 |
| 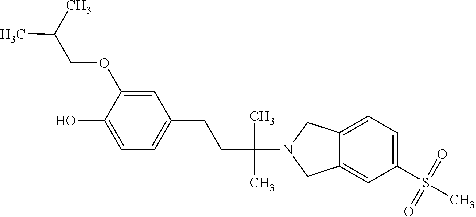 [SMILES: CC(C)COc1cc(CCC(C)(C)N2Cc3ccc(S(C)(=O)=O)cc3C2)ccc1O] | Ex. Cpd. No. 87 |
| 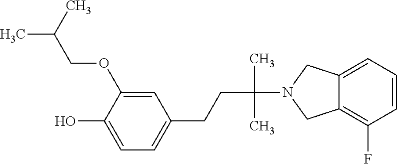 [SMILES: CC(C)COc1cc(CCC(C)(C)N2Cc3cccc(F)c3C2)ccc1O] | Ex. Cpd. No. 88 |
| 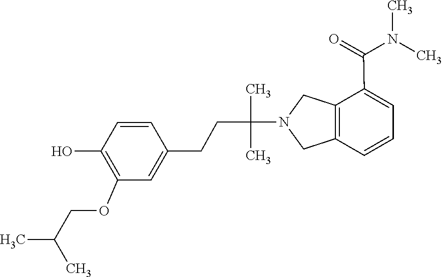 [SMILES: CC(C)COc1cc(CCC(C)(C)N2Cc3cccc(C(=O)N(C)C)c3C2)ccc1O] | Ex. Cpd. No. 87 |
| 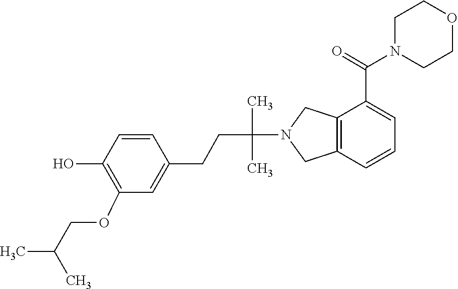 [SMILES: CC(C)COc1cc(CCC(C)(C)N2Cc3cccc(C(=O)N4CCOCC4)c3C2)ccc1O] | Ex. Cpd. No. 90 |
| 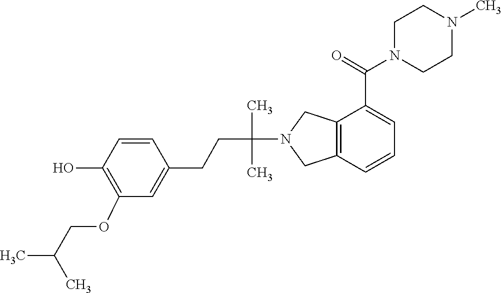 [SMILES: CC(C)COc1cc(CCC(C)(C)N2Cc3cccc(C(=O)N4CCN(C)CC4)c3C2)ccc1O] | Ex. Cpd. No. 91 |
| 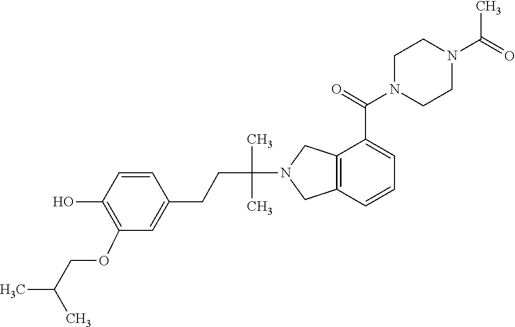 [SMILES: CC(=O)N1CCN(C(=O)c2cccc3c2CN(C(C)(C)CCc2ccc(O)c(OCC(C)C)c2)C3)CC1] | Ex. Cpd. No. 92 |
| 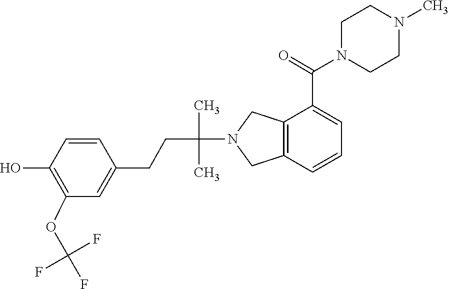 [SMILES: CN1CCN(C(=O)c2cccc3c2CN(C(C)(C)CCc2ccc(O)c(OC(F)(F)F)c2)C3)CC1] | Ex. Cpd. No. 93 |
| 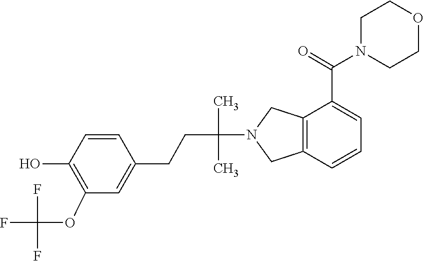 [SMILES: CC(C)(CCc1ccc(O)c(OC(F)(F)F)c1)N1Cc2cccc(C(=O)N3CCOCC3)c2C1] | Ex. Cpd. No. 94 |
| 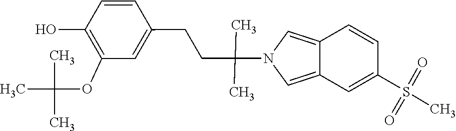 [SMILES: CC(C)(C)Oc1cc(CCC(C)(C)n2cc3ccc(S(C)(=O)=O)cc3c2)ccc1O] | Ex. Cpd. No. 95 |
| 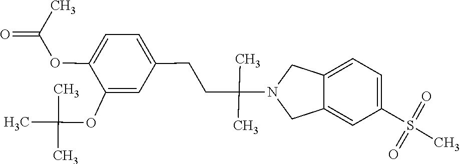 [SMILES: CC(=O)Oc1ccc(CCC(C)(C)N2Cc3ccc(S(C)(=O)=O)cc3C2)cc1OC(C)(C)C] | Ex. Cpd. No. 96 |
| 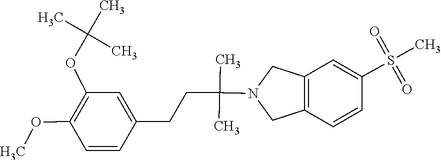 | Ex. Cpd. No. 97 |
| 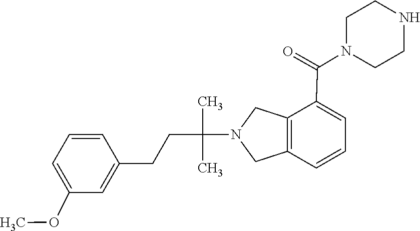 [SMILES: COc1cccc(CCC(C)(C)N2Cc3cccc(C(=O)N4CCNCC4)c3C2)c1] | Ex. Cpd. No. 98 |
| 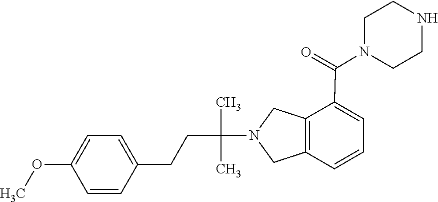 [SMILES: COc1ccc(CCC(C)(C)N2Cc3cccc(C(=O)N4CCNCC4)c3C2)cc1] | Ex. Cpd. No. 99 |
| 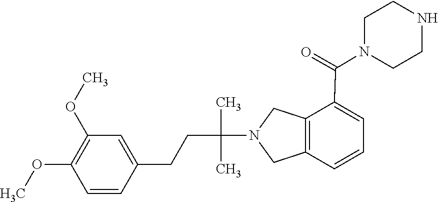 [SMILES: COc1ccc(CCC(C)(C)N2Cc3cccc(C(=O)N4CCNCC4)c3C2)cc1OC] | Ex. Cpd. 100 |
| 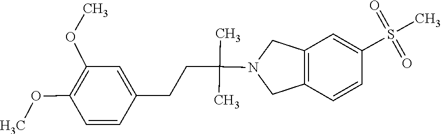 | Ex. Cpd. 101 |
| 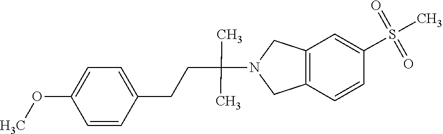 | Ex. Cpd. 102 |
| 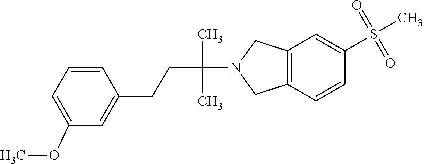 | Ex. Cpd. 103 |
| 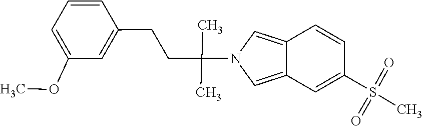 | Ex. Cpd. 104 |
| 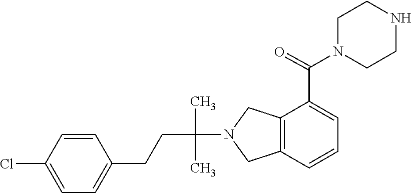 [SMILES: CC(C)(CCc1ccc(Cl)cc1)N1Cc2cccc(C(=O)N3CCNCC3)c2C1] | Ex. Cpd. 105 |
| 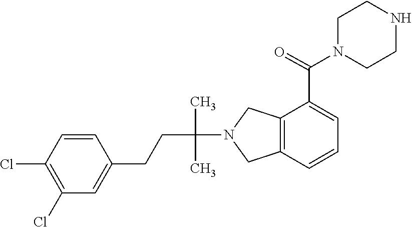 [SMILES: CC(C)(CCc1ccc(Cl)c(Cl)c1)N1Cc2cccc(C(=O)N3CCNCC3)c2C1] | Ex. Cpd. 106 |
| 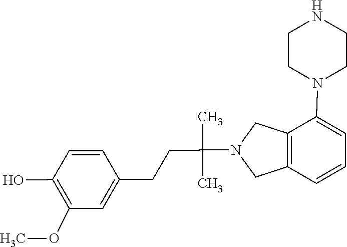 [SMILES: COc1cc(CCC(C)(C)N2Cc3cccc(N4CCNCC4)c3C2)ccc1O] | Ex. Cpd. 107 |
| 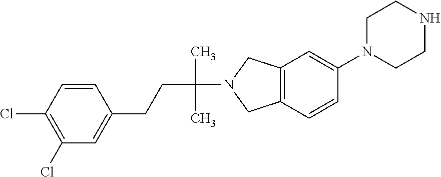 [SMILES: CC(C)(CCc1ccc(Cl)c(Cl)c1)N1Cc2ccc(N3CCNCC3)cc2C1] | Ex. Cpd. 108 |
| 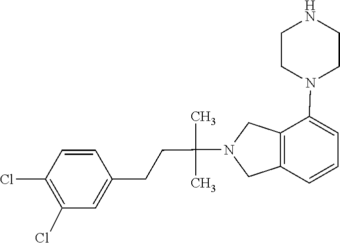 [SMILES: CC(C)(CCc1ccc(Cl)c(Cl)c1)N1Cc2cccc(N3CCNCC3)c2C1] | Ex. Cpd. 109 |
| 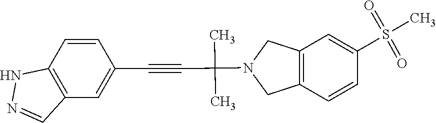 [SMILES: CC(C)(C#Cc1ccc2[nH]ncc2c1)N1Cc2ccc(S(C)(=O)=O)cc2C1] | Ex. Cpd. 110 |
| 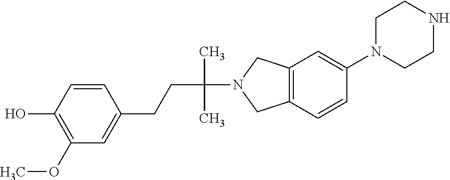 [SMILES: COc1cc(CCC(C)(C)N2Cc3ccc(N4CCNCC4)cc3C2)ccc1O] | Ex. Cpd. 111 |
| 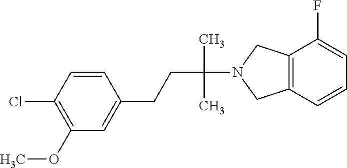 [SMILES: COc1cc(CCC(C)(C)N2Cc3cccc(F)c3C2)ccc1Cl] | Ex. Cpd. 112 |
| 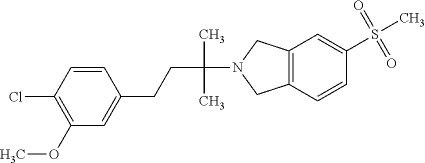 [SMILES: COc1cc(CCC(C)(C)N2Cc3ccc(S(C)(=O)=O)cc3C2)ccc1Cl] | Ex. Cpd. 113 |
| 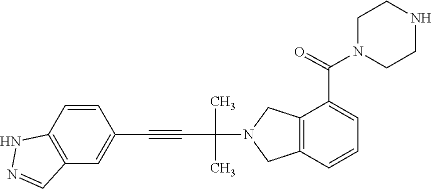 [SMILES: CC(C)(C#Cc1ccc2[nH]ncc2c1)N1Cc2cccc(C(=O)N3CCNCC3)c2C1] | Ex. Cpd. 114 |
| 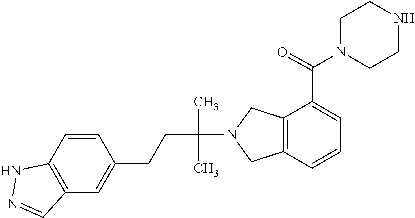 [SMILES: CC(C)(CCc1ccc2[nH]ncc2c1)N1Cc2cccc(C(=O)N3CCNCC3)c2C1] | Ex. Cpd. 115 |
| 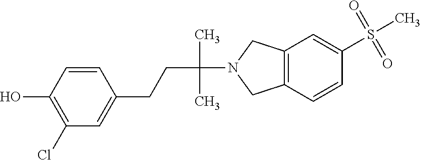 [SMILES: CC(C)(CCc1ccc(O)c(Cl)c1)N1Cc2ccc(S(C)(=O)=O)cc2C1] | Ex. Cpd. 116 |
| 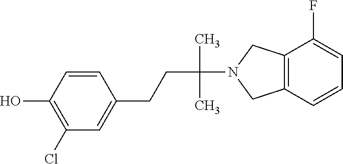 [SMILES: CC(C)(CCc1ccc(O)c(Cl)c1)N1Cc2cccc(F)c2C1] | Ex. Cpd. 117 |
| 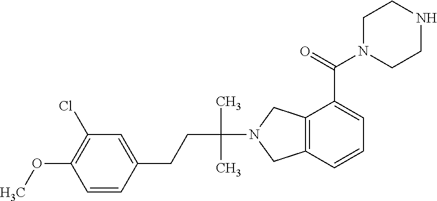 [SMILES: COc1ccc(CCC(C)(C)N2Cc3cccc(C(=O)N4CCNCC4)c3C2)cc1Cl] | Ex. Cpd. 118 |

### Example 4—Phase 1, Two-Part Single and Multiple Ascending Dose Study of CT1812

### Abstract

(CT1812) is a novel allosteric antagonist of the sigma-2 receptor complex that prevents and displaces binding of Aβ oligomers to neurons. By stopping a key initiating event in Alzheimer's disease (AD), this first-in-class drug candidate mitigates downstream synaptotoxicity and restores cognitive function in aged transgenic mouse models of AD.

A Phase 1, two-part single and multiple ascending dose study was conducted in 7 and 4 cohorts of healthy human subjects respectively. In Part A healthy, young subjects (<65 years old) received CT1812 doses ranging from 10-1120 mg (6:2 active to placebo (A:P) per cohort). In Part B, subjects were administered 280, 560, and 840 mg once daily for 14 days (8:2 A:P per cohort). An elderly cohort, age 65-75, was dosed at 560 mg once daily for 14 days (7:2 A:P). Serum concentrations of CT1812 in Part B were measured on day 3 and 14 and CSF concentrations on Day 7 or 9. Cognitive testing was performed in the healthy elderly cohort at baseline and at Day 14 of treatment.

Treatment with CT1812 was well tolerated in all cohorts. Adverse events were mild to moderate in severity and included headache and GI tract symptoms. Plasma concentrations of drug were dose proportional across two orders of magnitude with minimal accumulation over 14 days. Cognitive scores in the healthy elderly cohort were similar before and after treatment. CT1812 was well tolerated with single dose administration up to 1120 mg and with multiple dose administration up to 840 mg and 560 mg in healthy young and healthy elderly subjects, respectively. CT1812 is currently being studied in early phase 2 trials in patients with AD.

Cognition Therapeutics, Inc. (CogRx) has discovered a highly brain penetrant, first-in-class drug, Elayta™ (CT1812), that displaces Aβ oligomers (AβOs) bound to neuronal receptors at synapses. CT1812, a lipophilic isoindoline formulated as a fumarate salt, works similarly to a related class of compounds which have high affinity and specificity for the sigma-2 receptor complex, a key regulator of oligomer receptors. Binding allosterically to the sigma-2 receptor complex, this family of molecules destabilizes the AβO binding site, increasing the off-rate of AβOs, which are cleared into the CSF. In preclinical models, CT-family compound receptor occupancy at or exceeding 80% prevents downstream synaptotoxicity and restores memory in aged transgenic mouse models of AD.

### Study Design

A two-part Phase I, randomized, double-blind, placebo-controlled study of CT1812 was conducted in healthy young and elderly subjects: a single ascending dose (SAD)/food-effect study (Part A) and a multiple ascending dose (MAD) study (Part B). The primary endpoint was safety and tolerability. Secondary objectives included plasma pharmacokinetics (PK) in Parts A and B. CSF samples were also collected in the MAD study for analysis of PK and PD biomarkers. Cognitive testing was including in the elderly cohort in Part B as part of the safety assessment. Safety was assessed after completion of each cohort before ascending to the next dose level. The SAD/food-effect and MAD studies were conducted at Nucleus Network, Royal Alfred Hospital, Melbourne, Australia.

Part A was a single ascending dose cohort study in which healthy, young subjects (less than 65 years old) received one dose of study drug in the morning after an overnight fast. Cohort dosing started at 10 mg and increased to 30 mg, 90 mg, 180 mg, 450 mg, and 1120 mg in subsequent cohorts. Six drug-treated and two placebo-treated subjects were randomized in each cohort. A seventh cohort of six subjects each received a single 90 mg dose of drug 30 min. after a meal. Following completion of all safety assessments and blood draws for PK analyses, subjects were discharged on Day 3.

In Part B, healthy young subjects in each cohort received the same dose once daily for 14 days after overnight fasting. Cohort dosing started at 280 mg, followed by 560 mg and 840 mg in subsequent cohorts. In each cohort, eight subjects received drug and two received placebo. A fourth cohort of healthy elderly subjects (≥65 years old) received a 560 mg dose vs. placebo daily for 14 days (seven active, two placebo).

Subjects were dosed in the morning with 240 mL of water after an 8-hour fast, and remained in a semi-reclined position for 1 hour and fasting for 2 hours post-administration, except for the fed cohort in Part A. Subjects in each MAD cohort were confined to the clinical facility from check-in on Day 0 until the pharmacokinetic sample was collected on Day 16, 48 hours after administration of the last dose on Day 14. Subjects returned to the clinical facility for follow-up visits on Days 24 and 35.

### Participants

Healthy male and female subjects (determined by history, exam, and laboratory) were enrolled, with young subjects aged 18 to 64 years old and elderly subjects aged≥65 and ≤75. Female subjects must have been postmenopausal or surgically sterile. A history of acute/chronic hepatitis B or C and/or serology consistent with being a carrier of hepatitis B or HIV infection was exclusionary. All prescription, over-the-counter and herbal medications were prohibited within 10 days of study dosing (with the exception of nasal steroids, ocular medications, and paracetamol≤1000 mg/day at the discretion of the Investigator). Any contraindication to undergoing a lumbar puncture (LP) was also exclusionary for subjects undergoing CSF collection in Part B.

The Study protocol was approved by the Human Research Ethics Committee at the Alfred Hospital, Melbourne, Australia and was conducted in accordance with the Declaration of Helsinki and Good Clinical Practice guidelines. All subjects provided written informed consent before participating.

### Pharmacokinetic Assessments

In Part A, blood draws for assessment of PK parameters occurred pre-dose and at 15, 30, 45, 60, and 90 minutes post dose as well as at 2, 3, 4, 8, 12, 24, 36, and 48 hours post dose. Subjects in cohorts 5 and 6 had an additional sample drawn 72 hours after dosing. In Part B, blood samples for plasma PK analysis were taken on Day 1 at pre-dose and at 2 hours post-dose, on Day 3 at pre-dose and at 15, 30 and 45 minutes and 1, 2, 3, 4, 8, 12 and 24 hours post-dose, on Days 4, 6, 8 and 10 at pre-dose and at 1.5 hours post-dose and following the final dose on Day 14 at pre-dose and 15, 30 and 45 minutes and 1, 2, 4, 8, 12, 24, 36 and 48 hours post-dose. CT1812 concentrations in plasma samples were quantified using a validated liquid chromatography method with tandem mass spectrometric detection (LC-MS/MS).

Plasma concentrations for each dose level following single and repeated oral doses of CT1812 were used to determine PK parameters using noncompartmental methods, including: Cₘₐₓ—maximum concentration, Tₘₐₓ—time to maximum observed plasma drug concentration, AUC₀₋ₜ, AUC_{0-inf}, and AUC₀₋₂₄ (after multiple dosing)—area under the curve, CL/F—apparent drug clearance after an oral dose, λz—terminal phase rate constant, t½—terminal half-life, steady state concentration (Css), and time to reach steady state.

In Part B, cerebrospinal fluid samples were drawn from the 560 mg and 840 mg healthy young cohorts with a single spinal tap on day 7 or 9 of treatment at 1.5 hr after dose (approximate plasma Tₘₐₓ). CT1812 was quantitated in CSF using a validated LC-MS/MS method.

### Cognitive Testing

The Alzheimer's Disease Assessment Scale-Cognition Subscale (ADAS-Cog 13) and a cognitive battery (including the category fluency test, controlled word association test, WMS-R digit span, digit symbol substitution test, and Rey Auditory Verbal Learning Test) were administered to subjects in the elderly cohort at baseline and on Day 14.

### Safety Assessments

Safety variables, including incidence of adverse events (AEs), vital signs, clinical laboratory findings, 12-lead electrocardiographs (ECGs), physical examination, and affective and cognitive measures (Part B only), were summarized for all subjects who received study drug.

### Statistical Analysis

No formal statistical determination of cohort size was conducted, however the number of subjects used is considered sufficient to explore safety in an early clinical study. Pharmacokinetic parameters of plasma CT1812 were summarized by treatment, using descriptive statistics.

The analysis included the effect of food on bioavailability (Part A, Cohort 7 fed dose compared with Cohort 3 subjects administered the same dose in the fasted state) and the effect of age on CT1812 PK (Part B, Cohort 5 subjects aged at least 65 years [elderly] compared to Cohort 2 subjects aged up to 64 years [young]). This was assessed by analysis of variance (ANOVA) of log-transformed Cₘₐₓ, AUC₀₋₂₄, AUC₀₋₄₈, and/or AUC_{0-inf}, using a model with factors for treatment (fed status or age [young vs. elderly] status) and subject within sequence. Treatment mean differences and 90% confidence intervals of the log transformed PK parameters were back-transformed, to present the geometric least squares means ratios and 90% confidence limits. Determination of time to steady state for CT1812 in Part B was performed using Helmert Contrasts in ANOVA of pre-dose trough concentrations on Days 3, 4, 6, 7, 8, 9, 10, and 14, and the concentration at 24 hours post dose on Day 15. Dose proportionality was investigated using the power model, determined by regression of log-transformed parameters and dose level, Parameter=α*Dose^{β}.

### Results

### Demographics and Disposition of Subjects

A total of 93 subjects participated in the study. In the SAD phase, a total of 54 subjects were enrolled and randomized to treatment. Subjects were predominantly male (70%) and Caucasian (85%), with a median age of 26 years (range 19-55) (Table 7). In the MAD phase, a total of 39 subjects were enrolled and randomized to treatment. In the 3 young cohorts, subjects were predominantly male (77%) and Caucasian (87%), with a median age of 28.5 years (range 19-60). In the elderly cohort, 9 subjects were treated (7 CT1812, 2 placebo), as one subject withdrew prior to dosing. The elderly subjects were all Caucasian and 55% male, with a median age of 69 (range 64 to 73) years (Table 8).

TABLE 7
Summary of demographics of SAD cohort

| | | Fasted CT1812 | | | | | | | | | | | |
|---|---|---|---|---|---|---|---|---|---|---|---|---|---|
| | | 10 mg | | 30 mg | | 90 mg | | 180 mg | | 450 mg | | 1120 mg | |
| Parameter | Statistic | (N = 6) | | (N = 6) | | (N = 6) | | (N = 6) | | (N = 6) | | (N = 6) | |
| Age (Years) | N | 6 | | 6 | | 6 | | 6 | | 6 | | 6 | |
| | Mean | 28.7 | | 39.0 | | 23.2 | | 29.7 | | 25.0 | | 30.0 | |
| | SD | 5.5 | | 12.2 | | 3.0 | | 13.1 | | 3.1 | | 10.0 | |
| | Median | 30.5 | | 37.0 | | 22.5 | | 25.0 | | 25.0 | | 27.5 | |
| | Min | 21 | | 25 | | 21 | | 19 | | 21 | | 21 | |
| | Max | 35 | | 54 | | 29 | | 55 | | 30 | | 48 | |
| Gender | | | | | | | | | | | | | |
| Female | n (%) | 3 | (50%) | 3 | (50%) | 1 | (17%) | 1 | (17%) | 2 | (33%) | 2 | (33%) |
| Male | n (%) | 3 | (50%) | 3 | (50%) | 5 | (83%) | 5 | (83%) | 4 | (67%) | 4 | (67%) |
| Race | | | | | | | | | | | | | |
| Asian | n (%) | 1 | (17%) | | | | | 1 | (17%) | 1 | (17%) | 1 | (17%) |
| Caucasian | n (%) | 5 | (83%) | 5 | (83%) | 6 | (100%) | 4 | (67%) | 4 | (67%) | 5 | (83%) |
| Other | n (%) | | | 1 | (17%) | | | 1 | (17%) | 1 | (17%) | | |
| Ethnicity | | | | | | | | | | | | | |
| Hispanic or Latino | n (%) | | | | | 1 | (17%) | | | | | | |
| Non-Hispanic or Non- | n (%) | 6 | (100%) | 6 | (100%) | 5 | (83%) | 6 | (100%) | 6 | (100%) | 6 | (100%) |
| Latino | | | | | | | | | | | | | |

| | | | | | | | |
|---|---|---|---|---|---|---|---|
| Height (cm) | N | 6 | 6 | 6 | 6 | 6 | 6 |
| | Mean | 169.0 | 174.0 | 179.8 | 173.8 | 172.3 | 176.3 |
| | SD | 7.1 | 8.4 | 11.6 | 7.0 | 11.4 | 9.2 |
| | Median | 170.5 | 174.5 | 177.0 | 176.0 | 173.0 | 176.5 |
| | Min | 157 | 161 | 166 | 160 | 157 | 165 |
| | Max | 177 | 183 | 199 | 180 | 187 | 189 |
| Weight (kg) | N | 6 | 6 | 6 | 6 | 6 | 6 |
| | Mean | 69.45 | 76.70 | 83.92 | 77.55 | 70.87 | 79.00 |
| | SD | 10.43 | 11.57 | 11.04 | 7.48 | 10.02 | 7.88 |
| | Median | 68.50 | 76.35 | 86.35 | 78.45 | 69.35 | 79.25 |
| | Min | 54.6 | 60.3 | 70.6 | 68.5 | 60.0 | 67.3 |
| | Max | 86.9 | 91.7 | 94.7 | 85.9 | 86.3 | 89.6 |
| BMI (kg/m\hat{ }2) | N | 6 | 6 | 6 | 6 | 6 | 6 |
| | Mean | 24.33 | 25.22 | 26.22 | 25.68 | 23.88 | 25.52 |
| | SD | 3.40 | 2.02 | 4.65 | 2.39 | 2.69 | 3.18 |
| | Median | 23.55 | 25.05 | 26.25 | 26.65 | 23.60 | 24.95 |
| | Min | 20.1 | 22.9 | 20.2 | 21.6 | 21.3 | 21.6 |
| | Max | 29.0 | 27.7 | 31.2 | 28.0 | 28.6 | 31.0 |

| | | | | Fed | | | | | | | |
|---|---|---|---|---|---|---|---|---|---|---|---|
| | | Fasted CT1812 | | CT1812 | | Total | | Placebo | | Total | |
| | | Total | | 90 mg | | Active | | Pooled | | ALL | |
| Parameter | Statistic | (N = 36) | | (N = 6) | | (N = 42) | | (N = 12) | | (N = 54) | |
| Age (Years) | N | 36 | | 6 | | 42 | | 12 | | 54 | |
| | Mean | 29.3 | | 30.3 | | 29.4 | | 27.7 | | 29.0 | |
| | SD | 9.6 | | 9.6 | | 9.5 | | 7.7 | | 9.1 | |
| | Median | 26.0 | | 29.0 | | 26.5 | | 24.5 | | 26.0 | |
| | Min | 19 | | 21 | | 19 | | 20 | | 19 | |
| | Max | 55 | | 47 | | 55 | | 46 | | 55 | |
| Gender | | | | | | | | | | | |
| Female | n (%) | 12 | (33%) | 1 | (17%) | 13 | (31%) | 3 | (25%) | 16 | (30%) |
| Male | n (%) | 24 | (67%) | 5 | (83%) | 29 | (69%) | 9 | (75%) | 38 | (70%) |
| Race | | | | | | | | | | | |
| Asian | n (%) | 4 | (11%) | | | 4 | (10%) | | | 4 | (7%) |
| Caucasian | n (%) | 29 | (81%) | 5 | (83%) | 34 | (81%) | 12 | (100%) | 46 | (85%) |
| Other | n (%) | 3 | (8%) | 1 | (17%) | 4 | (10%) | | | 4 | (7%) |
| Ethnicity | | | | | | | | | | | |
| Hispanic or Latino | n (%) | 1 | (3%) | | | 1 | (%) | 2 | (17%) | 3 | (6%) |
| Non-Hispanic or Non- | n (%) | 35 | (97%) | 6 | (100%) | 41 | (%) | 10 | (83%) | 51 | (94%) |
| Latino | | | | | | | | | | | |

| | | | | | | |
|---|---|---|---|---|---|---|
| Height (cm) | N | 36 | 6 | 42 | 12 | 54 |
| | Mean | 174.2 | 177.5 | 174.7 | 179.2 | 175.7 |
| | SD | 9.3 | 10.6 | 9.4 | 7.8 | 9.2 |
| | Median | 175.5 | 180.0 | 176.0 | 180.5 | 176.5 |
| | Min | 157 | 162 | 157 | 160 | 157 |
| | Max | 199 | 190 | 199 | 188 | 199 |
| Weight (kg) | N | 36 | 6 | 42 | 12 | 54 |
| | Mean | 76.25 | 75.47 | 76.14 | 72.80 | 75.39 |
| | SD | 10.39 | 11.24 | 10.37 | 8.10 | 9.94 |
| | Median | 74.45 | 71.85 | 73.60 | 72.55 | 72.80 |
| | Min | 54.6 | 67.2 | 54.6 | 61.7 | 54.6 |
| | Max | 94.7 | 97.7 | 97.7 | 88.0 | 97.7 |
| BMI (kg/m\hat{ }2) | N | 36 | 6 | 42 | 12 | 54 |
| | Mean | 25.14 | 24.02 | 24.98 | 22.73 | 24.48 |
| | SD | 3.05 | 3.25 | 3.06 | 2.58 | 3.09 |
| | Median | 24.55 | 24.25 | 24.55 | 21.85 | 24.15 |
| | Min | 20.1 | 19.6 | 19.6 | 19.8 | 19.6 |
| | Max | 31.2 | 28.5 | 31.2 | 28.4 | 31.2 |

TABLE 8
Summary of demographics of MAD cohort

| | | | | | | | | | Fed | | | |
|---|---|---|---|---|---|---|---|---|---|---|---|---|
| | | Fasted CT1812 | | | | | | | CT1812 | Total | Placebo | Total |
| | | 10 mg | 30 mg | 90 mg | 180 mg | 450 mg | 1120 mg | Total | 90 mg | Active | Pooled | ALL |
| Parameter | Statistic | (N = 6) | (N = 6) | (N = 6) | (N = 6) | (N = 6) | (N = 6) | (N = 36) | (N = 6) | (N = 42) | (N = 12) | (N = 54) |
| Height | N | 6 | 6 | 6 | 6 | 6 | 6 | 36 | 6 | 42 | 12 | 54 |
| (cm) | Mean | 169.0 | 174.0 | 179.8 | 173.8 | 172.3 | 176.3 | 174.2 | 177.5 | 174.7 | 179.2 | 175.7 |
| | SD | 7.1 | 8.4 | 11.6 | 7.0 | 11.4 | 9.2 | 9.3 | 10.6 | 9.4 | 7.8 | 9.2 |
| | Median | 170.5 | 174.5 | 177.0 | 176.0 | 173.0 | 176.5 | 175.5 | 180.0 | 176.0 | 180.5 | 176.5 |
| | Min | 157 | 161 | 166 | 160 | 157 | 165 | 157 | 162 | 157 | 160 | 157 |
| | Max | 177 | 183 | 199 | 180 | 187 | 189 | 199 | 190 | 199 | 188 | 199 |
| Weight | N | 6 | 6 | 6 | 6 | 6 | 6 | 36 | 6 | 42 | 12 | 54 |
| (kg) | Mean | 69.45 | 76.70 | 83.92 | 77.55 | 70.87 | 79.00 | 76.25 | 75.47 | 76.14 | 72.80 | 75.39 |
| | SD | 10.43 | 11.57 | 11.04 | 7.48 | 10.02 | 7.88 | 10.39 | 11.24 | 10.37 | 8.10 | 9.94 |
| | Median | 68.50 | 76.35 | 86.35 | 78.45 | 69.35 | 79.25 | 74.45 | 71.85 | 73.60 | 72.55 | 72.80 |
| | Min | 54.6 | 60.3 | 70.6 | 68.5 | 60.0 | 67.3 | 54.6 | 67.2 | 54.6 | 61.7 | 54.6 |
| | Max | 86.9 | 91.7 | 94.7 | 85.9 | 86.3 | 89.6 | 94.7 | 97.7 | 97.7 | 88.0 | 97.7 |
| BMI | N | 6 | 6 | 6 | 6 | 6 | 6 | 36 | 6 | 42 | 12 | 54 |
| (kg/m\hat{ }2) | Mean | 24.33 | 25.22 | 26,22 | 25.68 | 23.88 | 25.52 | 25.14 | 24.02 | 24.98 | 22.73 | 24.48 |
| | SD | 3.40 | 2.02 | 4.65 | 2.39 | 2.69 | 3.18 | 3.05 | 3.25 | 3.06 | 2.58 | 3.09 |
| | Median | 23.55 | 25.05 | 26.25 | 26.65 | 23.60 | 24.95 | 24.55 | 24.25 | 24.55 | 21.85 | 24.15 |
| | Min | 20.1 | 22.9 | 20.2 | 21.6 | 21.3 | 21.6 | 20.1 | 19.6 | 19.6 | 19.8 | 19.6 |
| | Max | 29.0 | 27.7 | 31.2 | 28.0 | 28.6 | 31.0 | 31.2 | 28.5 | 31.2 | 28.4 | 31.2 |

| | | Age <= 64 | | | | | | Age >= 65 | | | Total | | |
|---|---|---|---|---|---|---|---|---|---|---|---|---|---|
| | | | | | ALL | | Total <= | | | Total >= | Total | Total | Grand |
| | | 280 mg | 560 mg | 840 mg | Active | Placebo | 64 | 560 mg | Placebo | 65 | Active | Placebo | Total |
| Parameter | Statistic | (N = 8) | (N = 8) | (N = 8) | (N = 24) | (N = 6) | (N = 30) | (N = 7) | (N = 2) | (N = 9) | (N = 31) | (N = 8) | (N = 39) |
| Age | N | 8 | 8 | 8 | 24 | 6 | 30 | 7 | 2 | 9 | 31 | 8 | 39 |
| (Years) | Mean | 28.3 | 40.9 | 30.1 | 33.1 | 24.3 | 31.3 | 67.7 | 71.5 | 68.6 | 40.9 | 36.1 | 39.9 |
| | SD | 7.7 | 17.1 | 4.4 | 12.0 | 4.9 | 11.5 | 3.3 | 2.1 | 3.4 | 18.2 | 22.2 | 18.9 |
| | Median | 27.5 | 42.0 | 29.5 | 29.0 | 23.5 | 28.5 | 68.0 | 71.5 | 69.0 | 31.0 | 27.0 | 30.0 |
| | Min | 20 | 19 | 25 | 19 | 19 | 19 | 64 | 70 | 64 | 19 | 19 | 19 |
| | Max | 44 | 60 | 39 | 60 | 31 | 60 | 73 | 73 | 73 | 73 | 73 | 73 |
| Gender | | | | | | | | | | | | | |
| Female | n (%) | 2 | 2 | 2 | 6 | 1 | 7 | 3 | 1 | 4 | 9 | 2 | 11 |
| | | (25%) | (25%) | (25%) | (25%) | (17%) | (23%) | (43%) | (50%) | (44%) | (29%) | (25%) | (28%) |
| Male | n (%) | 6 | 6 | 6 | 18 | 5 | 23 | 4 | 1 | 5 | 22 | 6 | 28 |
| | | (75%) | (75%) | (75%) | (75%) | (83%) | (77%) | (57%) | (50%) | (56%) | (71%) | (75%) | (72%) |
| Race | | | | | | | | | | | | | |
| Asian | n (%) | 2 | 1 | 1 | 4 | | 4 | | | | 4 | | 4 |
| | | (25%) | (13%) | (13%) | (17%) | | (13%) | | | | (13%) | | (10%) |
| Caucasian | n (%) | 6 | 7 | 7 | 20 | 6 | 26 | 7 | 2 | 9 | 27 | 8 | 35 |
| | | (75%) | (88%) | (88%) | (83%) | (100%) | (87%) | (100%) | (100%) | (100%) | (87%) | (100%) | (90%) |
| Ethnicity | | | | | | | | | | | | | |
| Hispanic or | n (%) | | | 1 | 1 | 1 | 2 | | | | 1 | 1 | 2 |
| Latino | | | | (13%) | (4%) | (17%) | (7%) | | | | (3%) | (13%) | (5%) |
| Non- | n (%) | 8 | 8 | 7 | 23 | 5 | 28 | 7 | 2 | 9 | 30 | 7 | 37 |
| Hispanic | | (100%) | (100%) | (88%) | (96%) | (83%) | (93%) | (100%) | (100%) | (100%) | (97%) | (88%) | (95%) |
| Non-Latino | | | | | | | | | | | | | |
| Height | N | 8 | 8 | 8 | 24 | 6 | 30 | 7 | 2 | 9 | 31 | 8 | 39 |
| (cm) | Mean | 176.4 | 176.0 | 173.3 | 175.2 | 176.2 | 175.4 | 171.6 | 168.0 | 170.8 | 174.4 | 174.1 | 174.3 |
| | SD | 8.2 | 11.8 | 9.2 | 9.5 | 6.9 | 9.0 | 9.2 | 9.9 | 8.9 | 9.4 | 7.9 | 9.0 |
| | Median | 178.5 | 180.5 | 173.0 | 177.0 | 176.5 | 177.0 | 171.0 | 168.0 | 171.0 | 176.0 | 173.5 | 175.0 |
| | Min | 162 | 153 | 156 | 153 | 168 | 153 | 156 | 161 | 156 | 153 | 161 | 153 |
| | Max | 185 | 187 | 186 | 187 | 184 | 187 | 184 | 175 | 184 | 187 | 184 | 187 |
| Weight | N | 8 | 8 | 8 | 24 | 6 | 30 | 7 | 2 | 9 | 31 | 8 | 39 |
| (kg) | Mean | 74.75 | 82.91 | 69.75 | 75.80 | 78.20 | 76.28 | 75.77 | 82.10 | 77.18 | 75.80 | 79.18 | 76.49 |
| | SD | 7.61 | 13.86 | 9.73 | 11.64 | 14.11 | 11.95 | 16.09 | 0.14 | 14.21 | 12.48 | 12.06 | 12.31 |
| | Median | 76.75 | 89.00 | 67.50 | 76.75 | 78.00 | 77.85 | 71.50 | 82.10 | 82.00 | 74.60 | 80.60 | 78.90 |
| | Min | 63.6 | 52.9 | 59.8 | 52.9 | 54.9 | 52.9 | 52.2 | 82.0 | 52.2 | 52.2 | 54.9 | 52.2 |
| | Max | 82.7 | 96.6 | 86.1 | 96.6 | 93.2 | 96.6 | 98.0 | 82.2 | 98.0 | 98.0 | 93.2 | 98.0 |
| BMI | N | 8 | 8 | 8 | 24 | 6 | 30 | 7 | 2 | 9 | 31 | 8 | 39 |
| (kg/m\hat{ }2) | Mean | 24.06 | 26.61 | 23.25 | 24.64 | 25.05 | 24.72 | 25.49 | 29.25 | 26.32 | 24.83 | 26.10 | 25.09 |
| | SD | 2.20 | 2.89 | 2.65 | 2.88 | 3.24 | 2.90 | 3.65 | 3.46 | 3.77 | 3.03 | 3.60 | 3.14 |
| | Median | 24.25 | 26.45 | 22.95 | 25.30 | 25.90 | 25.55 | 24.60 | 29.25 | 26.80 | 25.20 | 26.40 | 25.70 |
| | Min | 20.0 | 22.6 | 20.0 | 20.0 | 19.0 | 19.0 | 21.4 | 26.8 | 21.4 | 20.0 | 19.0 | 19.0 |
| | Max | 27.0 | 31.3 | 26.6 | 31.3 | 27.8 | 31.3 | 30.9 | 31.7 | 31.7 | 31.3 | 31.7 | 31.7 |

### Pharmacokinetic Results

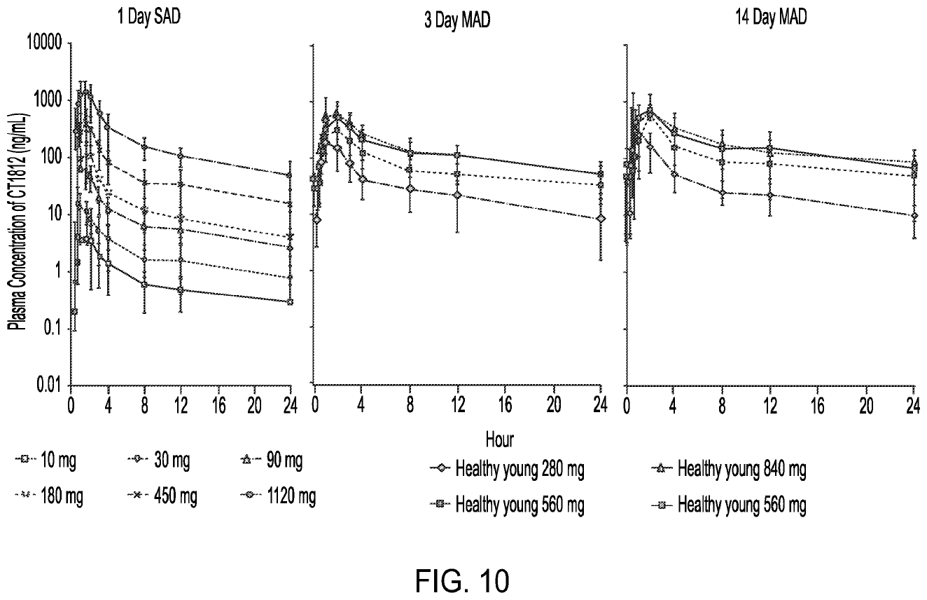

In part A (SAD), median CT1812 Tₘₐₓ values in plasma peaked at 0.88 to 1.5 hours (FIG. 10, Table 9). Cₘₐₓ and AUC increased slightly greater than dose proportionally in the single dose administration from 10 mg to 1120 mg (Table 9), and the clearance value CL/F showed a slight but steady downhill trend with increasing dose, consistent with the conclusion of a slightly greater than proportional increase in exposure with dose. The apparent mean half-life ranged from 11.1 to 14.0 hours. Following administration of a single 90 mg oral dose, the geometric least-squares mean for Cₘₐₓ, AUC₀₋₄₈ₕ, and AUC_{0-inf}, were approximately 40%, 20%, and 20% lower, respectively, at a dose of 90 mg under fed conditions compared to those observed in the fasted state. These differences were not considered clinically significant.

TABLE 9
Mean plasma pharmacokinetic parameters

| | | | | | AUC_{0-inf} | | |
|---|---|---|---|---|---|---|---|
| SAD CT1812 | | Dose | Cₘₐₓ ng/mL | Tₘₐₓ^{†} | hr · ng/mL | t_{1/2} hr^{§§} | CL/F |
| Dose (mg) | | Day | (CV %)* | hr (range) | (CV %) | (CV %) | (CV %) |
| Fasted | 10 mg | Day 1 | 5.07 | 1.50 | 30.6^{‡} | 12.0^{‡} | 662 |
| | (n = 6) | | (82%) | (0.50-2.00) | (67%) | (39%) | (118%) |
| | 30 mg | | 19.5 | 0.88 | 77.2 | 14.0 | 505 |
| | (n = 6) | | (68%) | (0.75-1.50) | (49%) | (29%) | (59%) |
| | 90 mg | | 109 | 1.25 | 305 | 12.1 | 340 |
| | (n = 6) | | (46%) | (0.75-2.00) | (43%) | (25%) | (38%) |
| | 180 mg | | 161 | 1.25 | 478 | 11.1 | 460 |
| | (n = 6) | | (59%) | (0.75-2.00) | (45%) | (31%) | (50%) |
| | 450 mg | | 504 | 1.50 | 1752 | 12.2 | 443 |
| | (n = 6) | | (69%) | (0.75-2.00) | (65%) | (14%) | (87%) |
| | 1120 mg | | 1462 | 1.50 | 6316 | 11.8 | 228 |
| | (n = 6) | | (54%) | (1.05-2.00) | (50%) | (29%) | (59%) |
| Fed | 90 mg | | 81.7 | 1.50 | 261 | 11.6 | 445 |
| | (n = 6) | | (94%) | (0.50-3.07) | (58%) | (37%) | (55%) |

| | | | | | AUC_{0-24 h} | | |
|---|---|---|---|---|---|---|---|
| MAD CT1812 | | Dose | Cₘₐₓ ng/mL | Tₘₐₓ^{†} | hr · ng/mL | t_{1/2} hr^{§§} | CLss/F |
| Dose (mg) | | Day | (CV %) | hr (range) | (CV %) | (CV %) | (CV %) |
| Age up to | 280 mg | Day 3 | 238 | 1.50 | 851 | 8.97^{§} | |
| 64 years | (n = 8) | | (77%) | (1.00-2.00) | (62%) | (19%) | |
| | | Day 14 | 431 | 0.88 | 1063 | 12.0 | 387 |
| | | | (95%) | (0.50-2.00) | (59%) | (35%) | (75%) |
| | 560 mg | Day 3 | 407 | 2.00 | 1943 | 8.56^{#} | |
| | (n = 8) | | (48%) | (0.75-3.03) | (41%) | (4.0%) | |
| | | Day 14 | 651 | 2.00 | 2811 | 12.3 | 296 |
| | | | (102%) | (1.00- 2.00) | (88%) | (20%) | (56%) |
| | 840 mg | Day 3 | 813 | 2.00 | 3612 | 9.39** | |
| | (n = 7) | | (79%) | (1.00-4.00) | (68%) | (13%) | |
| | 560 mg | Day 14 | 991 | 2.00 | 4989 | 8.93^{§} | 212^{‡‡} |
| | (n = 7) | | (84%) | (0.75-4.00) | (53%) | (25%) | (48%) |
| Age at | | Day 3 | 567 | 2.00 | 3330 | 8.83^{††} | |
| least 65 | | | (57%) | (1.00-4.00) | (49%) | (27%) | |
| years | | Day 14 | 853 | 1.00 | 4447 | 12.8 | 169^{‡‡} |
| | | | (64%) | (0.75-4.00) | (82%) | (20%) | (40%) |

*CV, Coefficient of variation;
^{†}Median;
^{†}n = 5;
^{§}n = 6;
^{#}n = 2;
**n = 5;
^{††}n = 4;
^{‡‡}n = 7;
^{§§}t_{1/2} values on Day 3 were determined over a maximum timeframe of 24 hours post dose

TABLE 10
Summary of Treatment-Emergent Adverse Events (TEAEs) by System Organ Class
and MedDRA Preferred Term Reported in More Than Two Subjects (SAD)

| Preferred Terms | | | | | | | | | | |
|---|---|---|---|---|---|---|---|---|---|---|
| reported in more | Number of Subjects (%) with Treatment-Emergent AE | | | | | | | | | |
| than 2 subjects | | | | | | | Fed | | | |
| System Organ | Fasted CT1812 | | | | | | CT1812 | Total | Placebo | All |
| Class, | 10 mg | 30 mg | 90 mg | 180 mg | 450 mg | 1120 mg | 90 mg | Active | Pooled | Subjects |
| Preferred Term | (n = 6) | (n = 6) | (n = 6) | (n = 6) | (n = 6) | (n = 6) | (n = 6) | (n = 42) | (n = 12) | (n = 54) |
| All TEAEs | 2 | 4 | | 2 | 4 | 4 | 2 | 18 | 2 | 20 |
| | (33%) | (67%) | | (33%) | (67%) | (67%) | (33%) | (43%) | (17%) | (37%) |
| Nervous system disorders | | | | | | | | | | |
| Headache | | 3 | | | 1 | (17%) | (17%) | 6 | 1 | 7 |
| | | (50%) | | | (17%) | 1 | 1 | (14%) | (8%) | (13%) |
| Gastrointestinal disorders | | | | | | | | | | |
| Nausea | | 1 | | | 2 | (50%) | | 6 | | 6 |
| | | (17%) | | | (33%) | 3 | | (14%) | | (11%) |

TABLE 11
TEAEs in MAD phase

| Preferred Terms | Number of Subjects (%) with at least one Treatment-Emergent AE | | | | | | | | |
|---|---|---|---|---|---|---|---|---|---|
| reported in more | | | | | | | | | |
| than 2 subjects | | | | | | | Total | | |
| System Organ | Age ≤ 64 years | | | | Age ≥ 65 years | | Total | Total | Grand |
| Class, | 280 mg | 560 mg | 840 mg | Placebo | 560 mg | Placebo | Active | Placebo | Total |
| Preferred Term | (n = 8) | (n = 8) | (n = 8) | (n = 6) | (n = 7) | (n = 2) | (n = 31) | (n = 8) | (n = 39) |
| All TEAES | 7 | 7 | 6 | 5 | 5 | 1 | 25 | 6 | 31 |
| | (88%) | (88%) | (75%) | (83%) | (71%) | (50%) | (81%) | (75%) | (79%) |
| Infections and infestations | | | | | | | | | |
| Upper respiratory | 5 | 1 | 2 | 2 | | | 8 | 2 | 10 |
| tract infection | (63%) | (13%) | (25%) | (33%) | | | (26%) | (25%) | (26%) |
| Nervous system disorders | | | | | | | | | |
| Dizziness | 1 | | 1 | | 1 | | 3 | | 3 |
| | (13%) | | (13%) | | (14%) | | (10%) | | (8%) |
| Headache | 2 | 1 | 4 | 1 | 3 | | 10 | 1 | 11 |
| | (25%) | (13%) | (50%) | (17%) | (43%) | | (32%) | (13%) | (28%) |
| Gastrointestinal disorders | | | | | | | | | |
| Dyspepsia | 1 | 1 | 1 | | | | 3 | | 3 |
| | (13%) | (13%) | (13%) | | | | (10%) | | (8%) |
| Nausea | 1 | | 1 | 1 | | | 2 | 1 | 3 |
| | (13%) | | (13%) | (17%) | | | (6%) | (13%) | (8%) |
| Vomiting | | | 2 | 1 | | | 2 | 1 | 3 |
| | | | (25%) | (17%) | | | (6%) | (13%) | (8%) |
| Musculoskeletal and connective tissue disorders | | | | | | | | | |
| Back pain | | | 1 | 1 | 1 | | 2 | (13%) | (8%) |
| | | | (13%) | (17%) | (14%) | | (6%) | 1 | 3 |
| General disorders and administration site conditions | | | | | | | | | |
| Catheter site | | 1 | 1 | 1 | | | 2 | 1 | 3 |
| phlebitis | | (13%) | (13%) | (17%) | | | (6%) | (13%) | (8%) |
| Injury, poisoning and procedural complications | | | | | | | | | |
| Procedural pain | | 4 | 2 | 2 | | | 6 | 2 | 8 |
| | | (50%) | (25%) | (33%) | | | (19%) | (25%) | (21%) |

In part B (MAD), CT1812 Tₘₐₓ values in plasma peaked at 0.88 to 2.0 hours (FIG. 10, Table 9). The PK parameters that reflect systemic exposure (Cₘₐₓ and AUC) increased slightly greater than dose proportionally from 280 to 840 mg (Table 9), and the clearance value CLss/F showed a slight but steady downhill trend with increasing dose, consistent with slightly greater than proportional increase in exposure with dose. Steady state was reached by approximately Day 3 or Day 4 of once daily dosing. Average terminal half-life was approximately 12 hours at steady state, which is consistent with that observed following a single dose in Part A.

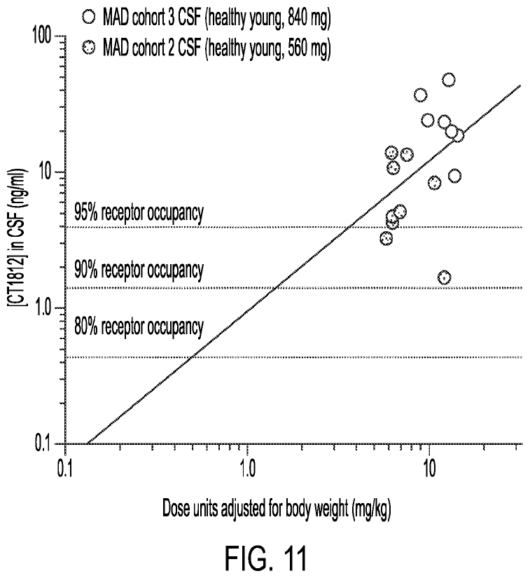

On day 3 of part B, geometric least-squares mean Cₘₐₓ and AUC₀₋₂₄ₕ values in the aged cohort (>65 years old) that received a daily dose level of 560 mg CT1812 were approximately 1.7- and 1.34-times higher compared to subjects under 64 years of age, respectively. The trend continued to Day 14 (steady state), with the Cₘₐₓ and AUC₀₋₂₄ₕ in the aged cohort (>65 years old) exceeding that of younger subjects (<64 years) by 1.6- and 1.5-times, respectively. CT1812 was measurable in CSF at 1.5 hr post dose on Day 7 to Day 9 in all subjects who received CT1812 daily at dose levels of 560 mg and 840 mg. Mean (±SD) levels of CT1812 in CSF were 8.0 (±4.3) and 23.3 (±15.6) ng/mL for 560 mg and 840 mg, respectively (FIG. 11).

### Safety Results:

### Safety Summary for SAD Phase

CT1812 was well tolerated across the single dose level range of 10 mg to 1120 mg. Treatment-emergent AEs were reported for 18 of 42 subjects (43%) following single dose administration of CT1812 and 2 of 12 subjects (17%) following administration of placebo (Table 10). There were no deaths or other serious adverse events (SAEs).

Most AEs (23 of 30, 77% of all AEs) were classified as mild in severity, with 7 AEs (23%) classified as moderate in severity (catheter site swelling, vomiting, nausea, vaccination site reaction, dysmenorrhea, and headache (2 AEs)). No AEs were classified as severe.

There were no subjects with clinically significant laboratory results in the SAD part of the study. All clinical laboratory results outside of the normal range were deemed not clinically significant. There were no marked differences by treatment (CT1812 vs placebo) or apparent dose-dependent trends in clinical laboratory results. No ECG parameters or changes were assessed as clinically significant.

### Safety Summary for MAD Phase

CT1812 was well tolerated across the multiple dose range 280 mg to 840 mg QD for 14 days. Treatment-emergent AEs were reported for 25 of 31 subjects (81%) following multiple dose administration with CT1812 and 6 of 8 subjects (75%) following multiple dose administration of placebo. One serious AE (SAE) was recorded in Part B (MAD); a subject receiving 840 mg CT1812 was hospitalized for a respiratory picornavirus infection deemed unrelated to study treatment. There were no deaths.

A total of 82 AEs were reported, with most (67 of 82, 82% of all AEs) classified as mild in severity, 14 AEs (17%) as moderate in severity, and one (1%) as severe. Qualitatively, there was no trend of increasing AE frequency with dose, with the exception of vomiting, where the two instances with active drug occurred at the 840 mg dose for an incidence of 25%. One subject in the placebo group experienced vomiting (17%).

Four subjects in the MAD study showed an increase in liver function tests below 3× the upper limit of normal (including one subject on placebo). One subject developed a rash while on study drug, which showed improvement after discontinuing CT1812. There were no marked differences by treatment (CT1812 vs placebo) or apparent dose dependent trends in clinical laboratory results. No ECG parameters or changes were assessed as clinically significant.

### Cognitive Testing

To ensure there were no deleterious effects on cognitive function in subjects given CT1812, cognitive testing was performed on the healthy elderly cohort receiving 560 mg of CT1812 per day, prior to initiation of dosing and at the end of the study. ADAS-COG 13 scores at day zero were 10.23±2.57 (SD) and were similar after day 14 of dosing (10.03±4.24). Results were also similar between day zero and day 14 on the other cognitive tests (Table 12).

TABLE 12
Cognitive Testing Results in Healthy Elderly with
repeated dosing of 560 mg for 14 days (N = 7)

| | Day 0 | Day 14 |
|---|---|---|
| Test | Mean (SD) | Mean (SD) |
| ADAS-COG 13 | 10.23 (2.57) | 10.03 (4.24) |
| WMS-R (Digit Span) | 17.9 (3.3) | 17.9 (3.3) |
| DSST | 78.3 (11.0) | 80 (9.6) |
| RAVLT | 17.3 (3.9) | 18.7 (3.1) |
| CFT | 18.4 (5.5) | 19.7 (2.4) |
| COWAT (Trial 1) | 11.3 (5.7) | 12.6 (4.5) |
| COWAT (Trial 2) | 9.6 (4.2) | 10.6 (5.1) |
| COWAT (Trial 3) | 14.6 (5.7) | 15.1 (3.7) |

### DISCUSSION

CT1812 was safe and well tolerated in healthy subjects over the dose range tested. In both parts (SAD and MAD), AEs were generally mild and included headache and GI disturbances. Plasma concentrations of drug increased slightly greater than dose proportionally across two orders of magnitude in Part A, and across a three-fold increase in dose in Part B. CT1812 levels assayed in CSF at peak plasma concentrations revealed dose-dependent increases in CT1812.

CT1812 levels in the CSF confirm that CT1812 penetrates the blood-brain barrier in humans, and extrapolations from mouse studies suggest that human doses administered once daily result in target concentrations that exceed the expected minimum concentration required to improve memory in mice (i.e. the concentration associated with >80% receptor occupancy). At the 560 mg dose, CSF CT1812 levels reached those associated with 95% receptor occupancy in transgenic mouse brain. At the 840 mg dose, CSF levels reached those associated with 98% receptor occupancy. Although no differences in CSF CT1812 concentrations are expected between Alzheimer's disease patients and aged matched cognitively normal individuals, future confirmatory studies will measure CSF levels of CT1812 in Alzheimer's disease patients. One additional cohort was given 90 mg of CT1812 after a meal to compare PK in the fed vs. fasted state, and there was no significant difference in CT1812 exposure based on AUC.

CT1812 is a novel, brain penetrant small molecule antagonist that prevents binding of AβOs to neuronal receptors. This drug candidate was safe and well tolerated in healthy subjects in this Phase 1a trial, mitigates downstream synaptotoxicity and restores memory to normal in aged transgenic mouse models of AD. CT1812 prevents and displaces AβOs through selective allosteric antagonism of the sigma-2 receptor complex, which, in turn, regulates the affinity of AβOs to their receptor protein. CT1812 decreases the affinity of bound AβOs to their receptor, causing their release and subsequent clearance from the brain. Importantly, this allosteric inhibition of binding by CT1812 is not likely be overcome by high AβO concentrations in later stages of the disease, as might occur with a competitive antagonist. As AβOs are likely neurotoxic throughout the course of AD, CT1812 may be effective in patients with symptomatic AD, whereas other therapeutics may be less effective in treating established disease.

The mechanism of CT1812 is unique among compounds tested in pre-clinical and clinical trials conducted to date. Other mechanistic approaches that have been tested include anti-aggregation agents, which work by preventing Aβ oligomer formation or disrupting oligomer structure once formed. However, clinical trials with these agents have not demonstrated efficacy to date. Another class of preclinical research compounds that prevents oligomer binding to the synaptic oligomer receptor, including cellular prion protein are under development.

AD is a complex disease that may ultimately require combination treatment directed at different targets. Some current therapeutic strategies (e.g. newer and more selective monoclonal antibodies directed against AD and tau) may eventually prove efficacious. However, these approaches will likely not completely ameliorate the negative effects of increasing concentrations of toxic Aβ oligomers that likely contribute to ongoing disease progression. CT1812, with its unique ability to decrease the affinity of bound Aβ oligomers to their receptors and clear them from the brain may have potential to address this therapeutic gap.

### Example 5—Assessing the Impact of CT1812 on Alzheimer's Disease Through Cerebral Spinal Fluid (CSF) Proteomics

Tandem-mass tag mass spectrometry (TMT-MS) was performed to enable over 2000 proteins to be detected in CSF of AD patients treated with CT1812 or placebo. A stringent filter was used to reduce noise. CSF was collected from CT1812 or placebo treated AD patients at baseline and at the end of the study (6 months) with reference standards. Samples were acquired from placebo and the drug and interventional cohort. Using biomarker differential-expression analysis, proteins that were differentially expressed between drug and placebo cohorts were identified (p<0.05).

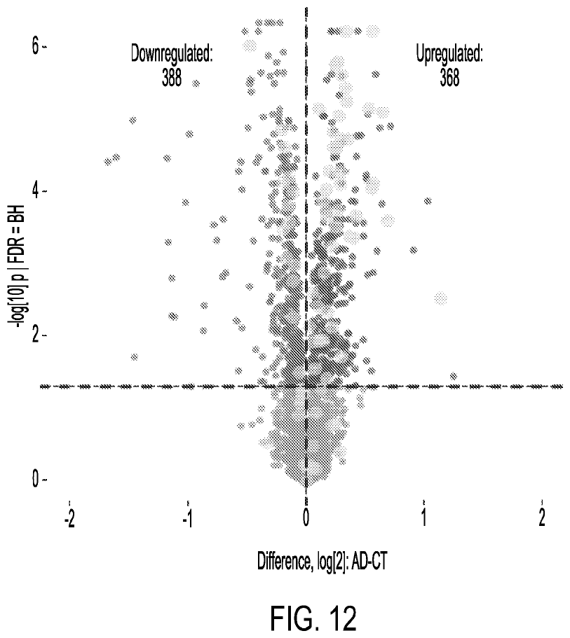

Assessment of CT1812 on biomarker expression was correlated with external traits (quantitative and clinical). Well-characterized priority biomarkers that were expected to be altered were significantly changed as expected (FDR p<0.05) and in a similar direction (e.g. MAPT, CHI3LI, NEFL) (FIG. 12). These results support the use of pooled AD and control CSF standards as suitable reference standards to compare to the SPARC trial. Comparison of CSF proteomes at baseline were measured. The goal was to understand if treatment effects normalize toward control.

CSF biomarker levels in SPARC Alzheimer's Disease patients were compared to an independent cohort of AD and control patients. At baseline, CSF AD biomarker levels in SPARC AD patients were similar to levels in CSF from AD patients from an independent cohort of AD patients (Emory ADRC) compared to that from non-demented (control) patients. This method enables benchmarking of the AD cohorts to an independent well-characterized AD and control cohort. Data indicate the SPARC patient cohort proteome is typical of AD, not control, cohort.

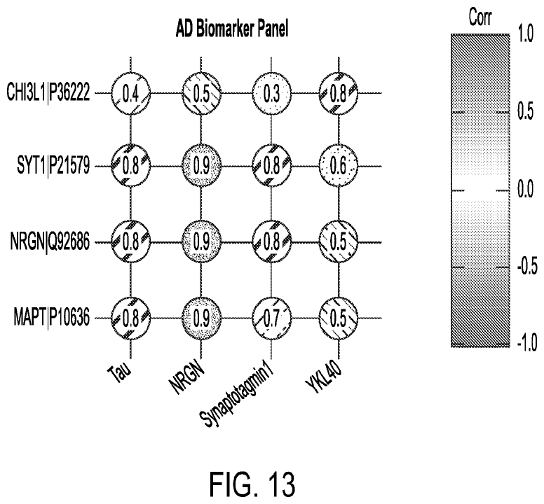

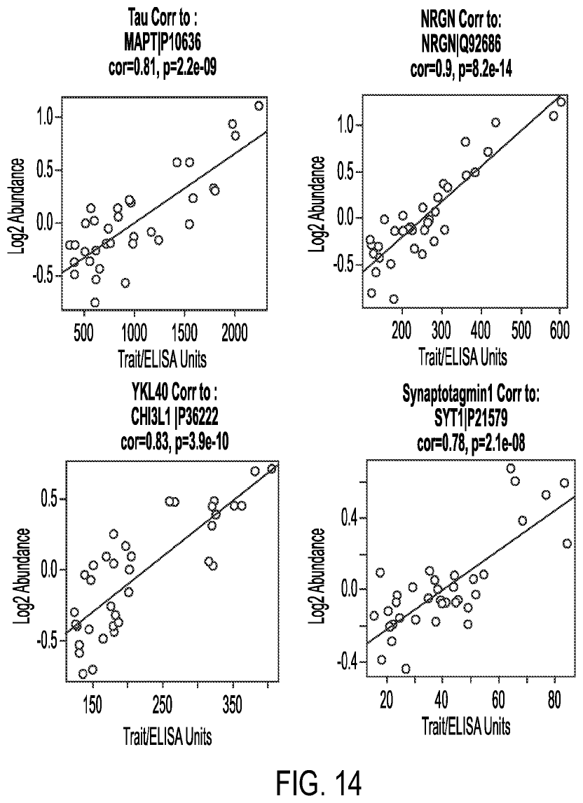

Comparison of levels of canonical AD biomarkers detected via proteomics TMT-MS to clinically validated quantitative assays. There were strong correlations, |r|>0.70, between TMT-MS values and clinically validated quantitative assays were observed across participants and timepoints (FIG. 13). This demonstrates that the method has good construct validity; and supports TMT-MS as a quantitative method (FIG. 14).

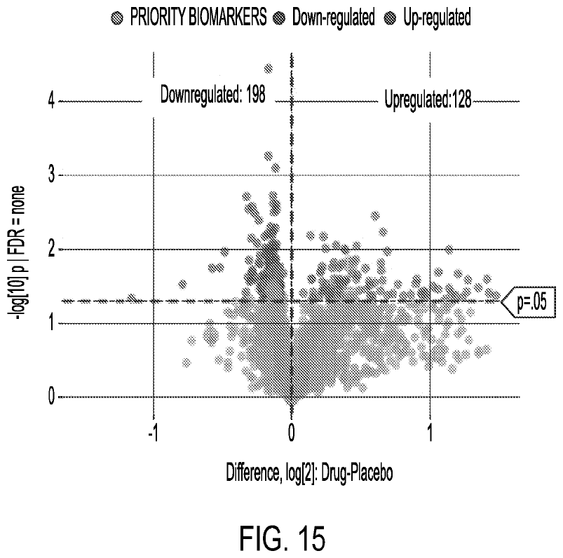

Differential expression analysis identified 326 significantly abundant proteins in CSF from CT1812 vs placebo (N=18, p<0.05). Three biomarkers were significantly decreased in patients treated with CT1812 for at least 6 months: CHI3L1 (YKL-40; an inflammatory protein); SMOC1 (a synaptic protein); and CLU (clusterin; a genetic risk factor for AD) (FIG. 15).

Pathway analyses identify proteins in key biologies are affected with CT1812 vs placebo in AD patients. The pathways statistically altered by CT1812 in SPARC overlap with in biological pathways discovered in our interim analysis of our SHINE clinical trial studying CT1812 in mild to moderate AD patients (Table 2).

All publications mentioned herein are incorporated by reference in their entirety. Nothing herein is to be construed as an admission that the disclosure is not entitled to antedate such disclosure by virtue of prior disclosure.

All features disclosed in the specification, including the abstract and drawings, and all the steps in any method or process disclosed, may be combined in any combination, except combinations where at least some of such features and/or steps are mutually exclusive. Each feature disclosed in the specification, including abstract and drawings, can be replaced by alternative features serving the same, equivalent or similar purpose, unless expressly stated otherwise. Thus, unless expressly stated otherwise, each feature disclosed is one example only of a generic series of equivalent or similar features. Various modifications of the disclosure, in addition to those described herein, will be apparent to those skilled in the art from the foregoing description. Such modifications are also intended to fall within the scope of the appended claims.

1.-32. (canceled)

33. A method of treating Alzheimer's disease in a subject in need thereof, comprising administering to the subject a therapeutically effective amount of a compound, or a pharmaceutically acceptable salt thereof, according to Formula I: 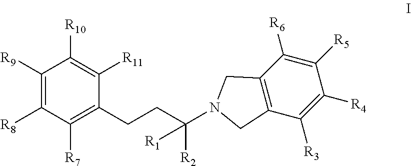
wherein:
R₁ and R₂ are each independently selected from H, C₁-C₆ alkyl, or CH₂OR′; where R′═H or C₁-C₆ alkyl;
R₃, R₄, R₅, and R₆ are each independently selected from H, C₁-C₆ alkyl, OH, OCH₃, OCH(CH₃)₂, OCH₂CH(CH₃)₂, OC(CH₃)₃, O(C₁-C₆ alkyl), OCF₃, OCH₂CH₂OH, O(C₁-C₆ alkyl)OH, O(C₁-C₆ haloalkyl), F, Cl, Br, I, CF₃, CN, NO₂, NH₂, C₁-C₆ haloalkyl, C₁-C₆ hydroxyalkyl, C₁₋₆ alkoxy C₁₋₆alkyl, aryl, heteroaryl, C₃₋₇ cycloalkyl, heterocycloalkyl, alkylaryl, heteroaryl, CO₂R′, C(O)R′, NH(C₁₋₄ alkyl), N(C₁₋₄ alkyl)₂, NH(C₃₋₇ cycloalkyl), NHC(O)(C₁₋₄ alkyl), CONR′₂, NC(O)R′, NS(O)ₙR′, S(O)ₙNR′₂, S(O)ₙR′, C(O)O(C₁₋₄ alkyl), OC(O)N(R′)₂, C(O) (C₁₋₄ alkyl), and C(O)NH(C₁₋₄ alkyl); where n=0, 1, or 2; R′ are each independently H, CH₃, CH₂CH₃, C₃-C₆ alkyl, C₁-C₆ haloalkyl; or optionally substituted aryl, alkylaryl, piperazin-1-yl, piperidin-1-yl, morpholinyl, heterocycloalkyl, heteroaryl, C₁₋₆ alkoxy, NH(C₁₋₄ alkyl), or NH(C₁₋₄ alkyl)₂, wherein optionally substituted group is selected from C₁-C₆ alkyl or C₂-C₇ acyl;
or R₃ and R₄, together with the C atom to which they are attached form a 4-, 5-, 6-7- or 8-membered cycloalkyl, aryl, heteroaryl, or heterocycloalkyl that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R³ and R⁴, or R⁴ and R⁵, are each independently selected from a bond, C, N, S, and O; or R₃ and R₄ are linked together to form a —O—C₁₋₂ methylene-O— group;
or R₄ and R₅, together with the C atom to which they are attached form a 4-, 5-, 6-7- or 8-membered cycloalkyl, aryl, heteroaryl, or heterocycloalkyl that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R³ and R⁴, or R⁴ and R⁵, are each independently selected from a bond, C, N, S, and O; or R₄ and R₅ are linked together to form a —O—C₁₋₂ methylene-O— group;
R₇, R₈, R₉, R₁₀, and R₁₁ are each independently selected from H, C₁-C₆ alkyl, OH, OCH₃, OCH(CH₃)₂, OCH₂CH(CH₃)₂, OC(CH₃)₃, O(C₁-C₆ alkyl), OCF₃, OCH₂CH₂OH, O(C₁-C₆ alkyl)OH, O(C₁-C₆ haloalkyl), O(CO)R′, F, Cl, Br, I, CF₃, CN, NO₂, NH₂, C₁-C₆ haloalkyl, C₁-C₆ hydroxyalkyl, C₁₋₆ alkoxy C₁₋₆alkyl, aryl, heteroaryl, C_{3.7} cycloalkyl, heterocycloalkyl, alkylaryl, heteroaryl, CO₂R′, C(O)R′, NH(C₁₋₄ alkyl), N(C₁₋₄ alkyl)₂, NH(C₃₋₇ cycloalkyl), NHC(O)(C₁₋₄ alkyl), CONR′₂, NC(O)R′, NS(O)ₙR′, S(O)ₙNR′₂, S(O)ₙR′, C(O)O(C₁₋₄ alkyl), OC(O)N(R′)₂, C(O) (C₁₋₄ alkyl), and C(O)NH(C₁₋₄ alkyl); where n=0, 1, or 2; R′ are each independently H, CH₃, CH₂CH₃, C₃-C₆ alkyl, C₁-C₆ haloalkyl, aryl, alkylaryl, piperazin-1-yl, piperidin-1-yl, morpholinyl, heterocycloalkyl, heteroaryl, C₁₋₆ alkoxy, NH(C₁₋₄ alkyl), or NH(C₁₋₄ alkyl)₂;
or R₇ and R₈, together with the N or C atoms to which they are attached form a 4-, 5-, 6- 7- or 8-membered cycloalkyl, aryl, heterocycloalkyl or heteroaryl group that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R⁹ and R¹⁰ are each independently selected from a bond, C, N, S, and O; or R₇ and R₈ are linked together to form a —O—C₁₋₂ methylene-O— group;
or R₈ and R₉, together with the N or C atoms to which they are attached form a 4-, 5-, 6- 7- or 8-membered cycloalkyl, aryl, heterocycloalkyl or heteroaryl group that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R⁹ and R¹⁰ are each independently selected from a bond, C, N, S, and O; or R₈ and R₉ are linked together to form a —O—C₁₋₂ methylene-O— group,
wherein each of the O, C₁₋₆ alkyl, C₁₋₆ haloalkyl, heteroaryl, aryl, heteroaryl, heterocycloalkyl, and cycloalkyl is optionally independently substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl and heterocycloalkyl;
with the proviso that the following compounds are excluded: 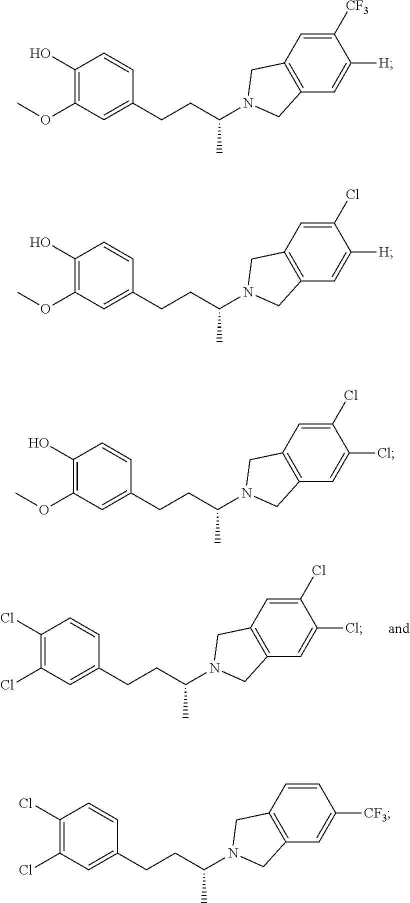
wherein the administration of the compound according to Formula I results in a decrease in the expression of at least one biomarker in the cerebral spinal fluid of the subject in need thereof, an increase in the expression of at least one biomarker in the cerebral spinal fluid of the subject in need thereof, or a combination of both;
wherein the at least one biomarker with decreased expression in the cerebral spinal fluid of the subject in need thereof is selected from the group consisting of Chitinase 3 Like 1 (CHI3L1), SPARC-Related Modular Calcium-Binding Protein 1 (SMOC 1), Clusterin (CLU), and any combination thereof.

34. The method of claim 33, wherein the subject has been diagnosed with Alzheimer's disease.

35. The method of claim 33, wherein the subject has been diagnosed with mild to moderate Alzheimer's disease.

36. The method of claim 33, wherein the subject is aged less than 50 years.

37. The method of claim 33, wherein the subject is aged between 50 and 80 years.

38. The method of claim 33, wherein the subject has elevated cerebral spinal fluid levels of a biomarker selected from the group consisting of Chitinase 3 Like 1 (CHI3L1), SPARC-Related Modular Calcium-Binding Protein 1 (SMOC 1), Clusterin (CLU), tyrosine 3-monooxygenase/tryptophan 5-monooxygenase activation protein gamma (YWHAG), major histocompatibility complex, class II, DR beta 1 (HLA-DRB-1), microtubule associated protein tau (MAPT), neurofilament light chain (NEFL), and any combination thereof, prior to administering the compound of Formula I.

39. The method of claim 33, wherein a decrease in expression of at least one biomarker selected from the group Chitinase 3 Like 1 (CHI3L1), SPARC-Related Modular Calcium-Binding Protein 1 (SMOC 1), Clusterin (CLU), and any combination thereof, after administering a therapeutically effective amount of a compound of Formula I is indicative of treatment success.

40. The method of claim 39, wherein the compound of Formula I is administered for at least about 6 months.

41. The method of claim 33, wherein the compound of Formula I is 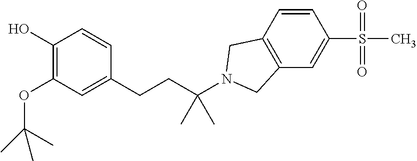 [SMILES: CC(C)(C)Oc1cc(CCC(C)(C)N2Cc3ccc(S(C)(=O)=O)cc3C2)ccc1O]
or a pharmaceutically acceptable salt thereof.

42. The method of claim 41, wherein the pharmaceutically acceptable salt is the fumarate salt.

43. The method of claim 41, wherein the therapeutically effective amount of the compound of Formula I is from about 0.0001 mg to about 1120 mg.

44. The method of claim 41, wherein the therapeutically effective amount of the compound of Formula I is about 100 mg to about 300 mg.

45. A method of treating Alzheimer's disease in a subject in need thereof, comprising administering to the subject a therapeutically effective amount of a compound, or a pharmaceutically acceptable salt thereof, according to Formula I: 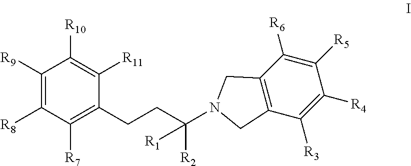
wherein:
R₁ and R₂ are each independently selected from H, C₁-C₆ alkyl, or CH₂OR′; where R′═H or C₁-C₆ alkyl;
R₃, R₄, R₅, and R₆ are each independently selected from H, C₁-C₆ alkyl, OH, OCH₃, OCH(CH₃)₂, OCH₂CH(CH₃)₂, OC(CH₃)₃, O(C₁-C₆ alkyl), OCF₃, OCH₂CH₂OH, O(C₁-C₆ alkyl)OH, O(C₁-C₆ haloalkyl), F, Cl, Br, I, CF₃, CN, NO₂, NH₂, C₁-C₆ haloalkyl, C₁-C₆ hydroxyalkyl, C₁₋₆ alkoxy C₁₋₆alkyl, aryl, heteroaryl, C₃₋₇ cycloalkyl, heterocycloalkyl, alkylaryl, heteroaryl, CO₂R′, C(O)R′, NH(C₁₋₄ alkyl), N(C₁₋₄ alkyl)₂, NH(C₃₋₇ cycloalkyl), NHC(O)(C₁₋₄ alkyl), CONR′₂, NC(O)R′, NS(O)ₙR′, S(O)ₙNR′₂, S(O)ₙR′, C(O)O(C₁₋₄ alkyl), OC(O)N(R′)₂, C(O) (C₁₋₄ alkyl), and C(O)NH(C₁₋₄ alkyl); where n=0, 1, or 2; R′ are each independently H, CH₃, CH₂CH₃, C₃-C₆ alkyl, C₁-C₆ haloalkyl; or optionally substituted aryl, alkylaryl, piperazin-1-yl, piperidin-1-yl, morpholinyl, heterocycloalkyl, heteroaryl, C₁₋₆ alkoxy, NH(C₁₋₄ alkyl), or NH(C₁₋₄ alkyl)₂, wherein optionally substituted group is selected from C₁-C₆ alkyl or C₂-C₇ acyl;
or R₃ and R₄, together with the C atom to which they are attached form a 4-, 5-, 6-7- or 8-membered cycloalkyl, aryl, heteroaryl, or heterocycloalkyl that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R³ and R⁴, or R⁴ and R⁵, are each independently selected from a bond, C, N, S, and O; or R₃ and R₄ are linked together to form a —O—C₁₋₂ methylene-O— group;
or R₄ and R₅, together with the C atom to which they are attached form a 4-, 5-, 6-7- or 8-membered cycloalkyl, aryl, heteroaryl, or heterocycloalkyl that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R³ and R⁴, or R⁴ and R⁵, are each independently selected from a bond, C, N, S, and O; or R₄ and R₅ are linked together to form a —O—C₁₋₂ methylene-O— group;
R₇, R₈, R₉, R₁₀, and R₁₁ are each independently selected from H, C₁-C₆ alkyl, OH, OCH₃, OCH(CH₃)₂, OCH₂CH(CH₃)₂, OC(CH₃)₃, O(C₁-C₆ alkyl), OCF₃, OCH₂CH₂OH, O(C₁-C₆ alkyl)OH, O(C₁-C₆ haloalkyl), O(CO)R′, F, Cl, Br, I, CF₃, CN, NO₂, NH₂, C₁-C₆ haloalkyl, C₁-C₆ hydroxyalkyl, C₁₋₆ alkoxy C₁₋₆alkyl, aryl, heteroaryl, C₃₋₇ cycloalkyl, heterocycloalkyl, alkylaryl, heteroaryl, CO₂R′, C(O)R′, NH(C₁₋₄ alkyl), N(C₁₋₄ alkyl)₂, NH(C₃₋₇ cycloalkyl), NHC(O)(C₁₋₄ alkyl), CONR′₂, NC(O)R′, NS(O)ₙR′, S(O)ₙNR′₂, S(O)ₙR′, C(O)O(C₁₋₄ alkyl), OC(O)N(R′)₂, C(O) (C₁₋₄ alkyl), and C(O)NH(C₁₋₄ alkyl); where n=0, 1, or 2; R′ are each independently H, CH₃, CH₂CH₃, C₃-C₆ alkyl, C₁-C₆ haloalkyl, aryl, alkylaryl, piperazin-1-yl, piperidin-1-yl, morpholinyl, heterocycloalkyl, heteroaryl, C₁₋₆ alkoxy, NH(C₁₋₄ alkyl), or NH(C₁₋₄ alkyl)₂;
or R₇ and R₈, together with the N or C atoms to which they are attached form a 4-, 5-, 6- 7- or 8-membered cycloalkyl, aryl, heterocycloalkyl or heteroaryl group that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R⁹ and R¹⁰ are each independently selected from a bond, C, N, S, and O; or R₇ and R₈ are linked together to form a —O—C₁₋₂ methylene-O— group;
or R₈ and R₉, together with the N or C atoms to which they are attached form a 4-, 5-, 6- 7- or 8-membered cycloalkyl, aryl, heterocycloalkyl or heteroaryl group that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R⁹ and R¹⁰ are each independently selected from a bond, C, N, S, and O; or R₈ and R₉ are linked together to form a —O—C₁₋₂ methylene-O— group, wherein each of the O, C₁₋₆ alkyl, C₁₋₆ haloalkyl, heteroaryl, aryl, heteroaryl, heterocycloalkyl, and cycloalkyl is optionally independently substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl and heterocycloalkyl;
with the proviso that the following compounds are excluded: 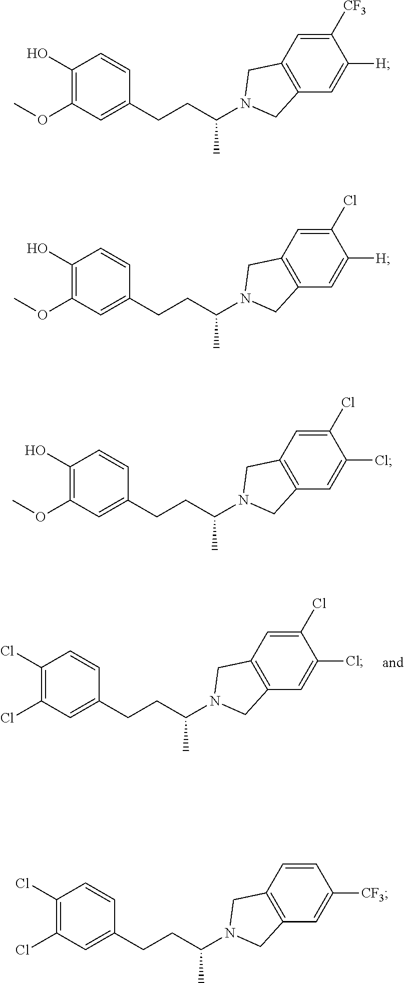
wherein the subject in nee thereof, prior to administration of the compound of Formula I, has a decrease in the expression of at least one biomarker in the cerebral spinal fluid of the subject in need thereof, an increase in the expression of at least one biomarker in the cerebral spinal fluid of the subject in need thereof, or a combination of both;
wherein the at least one biomarker with decreased expression in the cerebral spinal fluid of the subject in need thereof is selected from the group consisting of Chitinase 3 Like 1 (CHI3L1), SPARC-Related Modular Calcium-Binding Protein 1 (SMOC 1), Clusterin (CLU), and any combination thereof.

46. The method of claim 45, wherein the subject has been diagnosed with Alzheimer's disease.

47. The method of claim 45, wherein the subject has been diagnosed with mild to moderate Alzheimer's disease.

48. The method of claim 45, wherein the subject is aged less than 50 years.

49. The method of claim 45, wherein the subject is aged between 50 and 80 years.

50. The method of claim 45, wherein the subject has an MMSE score between about 18-26.

51. The method of claim 45, wherein the compound of Formula I is administered for at least about 6 months.

52. The method of claim 45, wherein the compound of Formula I is 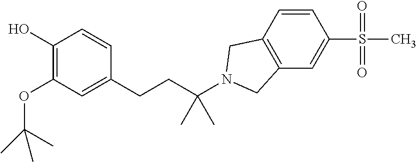 [SMILES: CC(C)(C)Oc1cc(CCC(C)(C)N2Cc3ccc(S(C)(=O)=O)cc3C2)ccc1O]
or a pharmaceutically acceptable salt thereof.

53. The method of claim 52, wherein the pharmaceutically acceptable salt is the fumarate salt.

54. The method of claim 52, wherein the therapeutically effective amount of the compound of Formula I is from about 0.0001 mg to about 1120 mg.

55. The method of claim 52, wherein the therapeutically effective amount of the compound of Formula I is about 100 mg to about 300 mg.
